(19) United States
(12) Patent Application Publication
(10) Pub. No.: US 2023/0407377 A1
(43) Pub. Date: Dec. 21, 2023

(54) CRISPR/CAS SCREENING SYSTEM MATERIALS AND METHODS

(71) Applicant: University of Copenhagen, Copenhagen K (DK)

(72) Inventors: Morten Frödin, Kongens Lyngby (DK); Yiyuan Niu, Copenhagen NV (DK); Catarina Ferreira A. Azevedo, Copenhagen NV (DK); Claus Storgaard Sørensen, Copenhagen S (DK)

(21) Appl. No.: 18/038,044

(22) PCT Filed: Dec. 3, 2021
(86) PCT No.: PCT/EP2021/084213

§ 371 Date: May 22, 2023

(30) Foreign Application Priority Data
Dec. 4, 2020 (EP) 20211801.4

(51) Int. Cl. C12Q 1/6827; C12N 15/10; C12N 9/22

(52) U.S. Cl. CPC C12Q 1/6827; C12N 15/102; C12N 9/22; C12N 2310/20

(57) ABSTRACT

The present disclosure relates to methods for assessing the effects of a mutation of interest in a cell. Herein are also disclosed systems for assessing the effects of a mutation of interest in a cell. The disclosure also provides host cells and host cell populations comprising the system.

### TECHNICAL FIELD

The present invention relates to methods and systems for screening and/or analysing the effect of genetic sequence variants on phenotypic parameters of cells. The genetic variants may be introduced in the genome using a gene editing system such as the CRISPR/Cas9 gene editing system.

### BACKGROUND

In all branches of life science and medicine, a need exists for assays that can determine how genetic sequence variants affect phenotypic parameters of cells, such as proliferation, survival, motility, metabolism or differentiation. A large need exists for example in the field of functional genetic diagnostics, personalized medicine and, personalised drug development. Thus, millions of sequence variants in thousands of genes in millions of patients are nowadays being revealed to doctors by routine patient DNA sequencing. But in the large majority of cases, doctors do not know if the variant is benign or pathogenic, even though this knowledge is essential for diagnosis and clinical management, which could involve drugs tailed to the disease-causing gene (called personalised medicine). Functional genetic diagnosis and testing for drug responsiveness would be the gold standard to solve these questions. Unfortunately, the currently available technologies for this are very laborious, time consuming and expensive and, consequently rarely used in the clinic. As another example, pharma uses huge resources to develop drugs against mutated genes (targeted therapies) or drugs that specifically act on cells with a mutation, but having a target other than the mutated gene (synthetic lethal therapies). Again, the availability of functional genetic model cell systems harbouring desired mutations for drug testing and screening would vastly accelerate the development of such therapies, but the current technologies for generation of genetic model cell systems impede progress. While oncology is the single field with the biggest unmet need for functional genetics, the demand is rapidly on the rise in other major diseases like diabetes/metabolic disorders, inflammation and neurological disease. Furthermore, in humans over 10,000 different monogenetic diseases exist, many of which would benefit tremendously from improved functional genetics methods for diagnosis, treatment and drug development.

The need to determine how a specific genotype impacts cell phenotype, however, is universal in life science and biotech. For instance, functional genetics may determine how genetic variants affect plant cell growth and biosynthesis or response to environmental factors, like drought, heat and pathogens. In bioproduction, functional genetic techniques may be used to improve quantity and quality of products derived from cellular organisms, be it animals, plants, yeast or bacteria.

Gene editing on populations of cells rank as the most powerful and commonly used method to assess the functional consequence of a mutation, reflected by the Nobel prize in chemistry 2020 awarded to the inventors or CRISPR/Cas9 editing. Accordingly, gene editing technologies have during the past decade become the centre of development and innovation in the life sciences, with aims to further investigate how different genotypes influence the phenotype of cells, tissues and organisms. However, a commonly encountered uncertainty in the assessment of the functional cellular effect of an induced mutation is to which extent the experimental design and practice affects the results.

In conventional knock-in experiments using gene-editing technologies, a mutation is introduced in the genome of a cell, the cell is expanded to a clonal cell population, which is finally analysed to determine the effect of the mutation. In this approach, the resulting cell behaviour may be influenced by many factors including transfection toxicities, off-target effects of the editing tool, unwanted selection of traits in the clone expansion step, as well as heterogeneity in the engineered cell population, to name a few. For example, a benign cancer mutation may show apparent loss-of-function effects on cellular fitness due to off-target effects on another genomic locus affecting cell proliferation. Alternatively, the clone(s) selected for analysis may incidentally be slow growing due to clonal variability (heterogeneity) in the cell line studied; another false positive result.

To address these multiple shortcomings, many repeat experiments and individually designed controls are necessary, all of which translates to high labour and material costs, and a lengthy time-to-results. Lengthy time-to-results is also inherent to the process due to the step of cell clone generation. Finally, if the mutation interferes with cell viability, it may not be possible to generate clones for study of the mutation.

There thus exists an urgent need for improved methods that can overcome these limitations.

### SUMMARY

As outlined above, conventional techniques aiming to elucidate the effects of specific genetic mutations in a cell are associated with a number of drawbacks that include a high propensity for false positive results. Consequently, these methods require appropriate countermeasures that result in a high amount of labour, time and materials being required to assess the effects of each individual mutation. Furthermore, the generation of clonal cell lines for analysis is inherently lengthy and requires that the mutation allows clone expansion.

Interestingly, the present invention provides methods that are accurate, fast, simple, cost-efficient and scalable for introducing one or more mutations of interest in a cell population and determining the effect of the variant on a broad range of cell parameters, such as proliferation and/or survival, within a short timeframe of one to a few weeks for complex cell lines, such as human cells. The use of an internal control containing one or more synonymous mutations at the same, or nearby position as the mutations of interest, and the analysis of hundreds of knock-in cells simultaneously will ensure that the observed differences in cell behaviour are only due to the mutations of interest. In one design of the PCR or next-generation sequencing (NGS) analysis of the introduced mutations, primers are designed to anneal outside the region substantially similar to the oligonucleotides introduced, allowing absolute frequencies to be calculated such that it can be controlled that statistically sufficient numbers of mutant cells underlie the results. A preferred design of the PCR/NGS analysis of the introduced mutations allows that absolute frequencies can be calculated such that it can be controlled that statistically sufficient numbers of mutant cells underlie the results

Another internal control for neutral mutations validates that they are truly neutral and not false negatives due to improper assay functioning. Performing the methods on a cell population eliminates the need for lengthy clone generation and allows for the study of mutations that interfere with clone generation. Finally, a flexible design allows to study the interaction of mutations of interest with cell environmental factors, such as drugs, stresses or pathogens. In this sense, the present methods provide a fast, reliable and versatile indication of what phenotypic effect a mutation of interest will have.

The invention is as defined in the claims.

Herein is provided a method for assessing the effects of a mutation of interest in a cell, said method comprising the steps of:
i) providing a cell population comprising a target nucleic acid sequence;
ii) introducing in at least some of the cells of the cell population:
   a) a nuclease or a polynucleotide encoding said nuclease, wherein the nuclease is capable of generating one or more single-strand breaks (SSBs) or double-strand breaks (DSBs) in a target nucleic acid sequence, and targeting means directing the nuclease to the target nucleic acid sequence, whereby the nuclease is capable of binding to a binding region of the target nucleic acid sequence, and whereby the nuclease is capable of generating one or more single-stranded breaks (SSBs) or double-strand breaks (DSBs) in the target nucleic acid sequence;
   b) a first oligonucleotide comprising a mutation of interest, preferably a non-silent mutation, preferably a non-synonymous mutation, wherein said non-synonymous mutation introduces a change in the encoded amino acid sequence compared to the amino acid sequence encoded by the target nucleic acid sequence, and otherwise identical to or complementary to said target nucleic acid sequence, wherein said mutation of interest preferably lies within the binding region of said nuclease; and
   c) a second oligonucleotide comprising a synonymous mutation, wherein said synonymous mutation preferably lies within the binding region of said nuclease, and wherein said synonymous mutation introduces no change in the encoded amino acid sequence compared to the amino acid sequence encoded by the target nucleic acid sequence;
   whereby said first oligonucleotide or said second oligonucleotide is integrated in, or copied into, the target nucleic acid sequence of at least some of the cells, thereby obtaining a mixed population of cells comprising cells in which the mutation of the first or the second oligonucleotides has not been introduced, cells in which only the mutation of the first oligonucleotide has been introduced, and cells in which only the mutation of the second oligonucleotide has been introduced;
iii) incubating the mixed population of cells in a medium for a determined duration, under conditions allowing a parameter of interest to be monitored, wherein the parameter of interest is a temporal parameter and/or a spatial parameter;
iv) determining the effect of the mutation of interest on the parameter of interest, wherein:
   A. if the parameter of interest is a temporal parameter, determining the effect of the mutation of interest on the parameter of interest comprises the steps of:
      v) determining an initial ratio of cells in which the mutation of interest has been introduced in the target nucleic acid sequence to cells in which the synonymous mutation has been introduced in the target nucleic acid sequence, wherein the initial ratio of cells is determined at an initial time point; determining a subsequent ratio of cells in which the mutation of interest has been introduced in the target nucleic acid sequence to cells in which the synonymous mutation has been introduced in the target nucleic acid sequence, wherein the subsequent ratio of cells is determined at a subsequent time point; and determining a change in ratio between the initial ratio and the subsequent ratio; and
      vi) correlating said change in ratio to the parameter of interest,
   and/or
   B. if the parameter of interest is a spatial parameter, determining the effect of the mutation of interest on the parameter of interest comprises the steps of:
      v) defining and/or spatially separating subpopulations of cells on the basis of said spatial parameter of interest in each subpopulation, preferably wherein the spatial parameter of interest is different in each subpopulation;
      vi) determining, for each subpopulation, a ratio of cells in which the mutation of interest has been introduced in the target nucleic acid sequence to cells in which the synonymous mutation has been introduced in the target nucleic acid sequence; and
      vii) correlating said ratio to the measured spatial parameter of interest for each subpopulation;
         thereby assessing the effect of the mutation on the parameter of interest.

Also provided herein is a system comprising:
i. a nuclease or a polynucleotide encoding said nuclease, wherein the nuclease is capable of generating one or more single-strand breaks (SSBs) or double-strand breaks (DSBs) in a target nucleic acid sequence, and targeting means directing the nuclease to the target nucleic acid sequence, whereby the nuclease is capable of binding to a binding region of the target nucleic acid sequence, and whereby the nuclease is capable of generating one or more single-stranded breaks (SSBs) or double-strand breaks (DSBs) in the target nucleic acid sequence;
ii. a first oligonucleotide comprising a mutation of interest, preferably a non-silent mutation, preferably a non-synonymous mutation, wherein said non-synonymous mutation introduces a change in the encoded amino acid sequence compared to the amino acid sequence encoded by the target nucleic acid sequence, and otherwise identical to or complementary to said target nucleic acid sequence, wherein said mutation of interest preferably lies within the binding region of said nuclease; and
iii. a second oligonucleotide comprising a synonymous mutation, wherein said synonymous mutation preferably lies within the binding region of said nuclease, and wherein said synonymous mutation introduces no change in the encoded amino acid sequence compared to the amino acid sequence encoded by the target nucleic acid sequence.

Additionally provided herein is a host cell or host cell population comprising the system as described herein.

Also provided herein is use of a system as described herein in a method for assessing the effects of a mutation of interest in a cell, wherein the method is as described herein.

### DESCRIPTION OF DRAWINGS

FIG. 1 shows the principle of the method. Step 1/Day 0: Introduce by co-transfection in some of the cells of a cell population the editing cassette comprising: 1) A genome editing nuclease that cuts the genomic site to be mutated; 2) An oligonucleotide with mutation of interest (MUT); and 3) An oligonucleotide with synonymous mutation (WT*). Step 2/Initial time point: Determine an initial ratio of cells in which mutation of interest has been introduced in target site to cells in which synonymous WT* mutation has been introduced in target site, e.g. by genomic PCR of the mutation site and NGS of PCR products. Step 3/Subsequent time point: Determine a subsequent ratio of cells in which mutation of interest has been introduced in target site to cells in which synonymous WT* mutation has been introduced in target site, e.g. by genomic PCR of mutation site and NGS of PCR products. Step 4/Result: Determine the change in ratio between the initial ratio and the subsequent ratio. In this example, the ratio decreased over time, demonstrating that the mutation of interest decreases cell proliferation and/or survival.

FIG. 2 shows cultures of human breast epithelial MCF10A-BRCA2+/− cells expressing SpCas9 that were transfected with synthetic gRNA/tracrRNA targeting genomic sites in BRCA2 overlapping the sites to be mutated along with an oligonucleotide comprising the mutation of interest (MUT), which was as either a known benign or a known pathogenic variant, and an oligonucleotide comprising a synonymous mutation (WT*). At Day 2 after transfection, an aliquot of each culture was analysed by PCR amplification of the genomic target site and NGS of the PCR product to determine the ratio of MUT to WT* alleles in the cell population. This analysis was repeated on Day 12. Data represent ratios normalized to the value at Day 2 and are means of 3 independent experiments +/−SD. Two-tailed unpaired t-tests were used to calculate the significance in all cases. NS=not significant. **** indicates p<0.0001.

FIG. 3 shows cultures of human breast epithelial MCF10A-BRCA2+/− cells expressing SpCas9 that were transfected with synthetic gRNA/tracrRNA targeting genomic sites in BRCA2 overlapping the sites to be mutated along with an oligonucleotide comprising the mutation of interest (MUT), which was as either a neutral variant (D946V) or loss-of-function variants (I2627N, Y2660C), and an oligonucleotide comprising a synonymous mutation (WT*). At Day 2 after transfection, an aliquot of each culture was analysed by PCR amplification of the genomic target site and NGS of the PCR product to determine the ratio of MUT to WT* alleles in the cell population. Thereafter, cells were cultured in the absence or presence of 2 nM Talazoparib (a PARP inhibitor) until Day 12, whereafter the mutation analysis was repeated. Data represent ratios

normalized to the value at Day 2 and are means of 3 independent experiments +/−SD. Two-tailed unpaired t-tests were used to calculate the significance in all cases. NS=not significant. ** indicates p<0.01. **** indicates p<0.0001.

FIG. 4 shows cultures of human breast epithelial MCF10A-BRCA2+/− cells expressing SpCas9 that were transfected with synthetic gRNA/tracrRNA targeting genomic sites in BRCA2 overlapping the sites to be mutated along with an oligonucleotide comprising the mutation of interest (MUT), which was as either a known benign variant (N289H) or a variant of unknown significance (D946V), and an oligonucleotide comprising a synonymous mutation (WT*). At Days 2 and 12 after transfection, an aliquot of each culture was analysed by PCR amplification of the genomic target site and NGS of the PCR product to determine (A) the ratio of MUT to WT* alleles in the cell population or (B) the frequency of frameshifting indel mutations in the cell population. In (A) and (B) data are means of 3 independent experiments +/−SD. Two-tailed unpaired t-tests were used to calculate the significance in all cases. *** indicates p<0.001.

FIG. 5 shows cultures of human breast epithelial MCF10A cells expressing SpCas9 that were transfected with synthetic gRNA/tracrRNA targeting a genomic site overlapping the site to be mutated along with an oligonucleotide comprising the oncogenic (gain-of-function) mutation of interest (MUT), i.e. H1047R in PIK3CA and an oligonucleotide comprising a synonymous mutation (WT*). At Day 2 after transfection, an aliquot of the cell culture was analysed by PCR amplification of the genomic target site and NGS of the PCR product to determine the ratio of MUT to WT* alleles in the cell population. Next, the cells were cultured under serum- and growth factor-deprived conditions until Day 13, whereafter the analysis was repeated. Data represent ratios normalized to the value at Day 2 and are means of 2 independent experiments +/−range. A two-tailed unpaired t-test was used to calculate the significance. ** indicates p<0.01.

FIG. 6 shows cultures of human lung cancer H358 cells that were nucleofected with SpCas9 protein and synthetic gRNA/tracrRNA targeting the oncogenic G12C mutation in KRAS present in these cells along with an oligonucleotide comprising the mutation of interest (MUT), which was a correction to wild-type G (12G*), and an oligonucleotide comprising a synonymous mutation (120*). At Day 2 after transfection, an aliquot of the cell culture was analysed by PCR amplification of the genomic target site and NGS of the PCR product to determine the ratio of 12G* versus 12C* alleles in the cell population. Next, cells were either (A) cultured for up to 18 days in vitro with determination of 12G* to 12C* ratios at the indicated time points or (B) xenografted into nude mice and the 12G* to 12C* ratio was determined in tumors after 12 days. Data represent ratios normalized to the value at Day 2. (B) represent data from 3 independent experiments +/−SD. A two-tailed unpaired t-test was used to calculate the significance. **** indicates p<0.0001.

FIG. 7 shows use of the method to determine mechanisms of drug resistance. (A) AMG 510 is an inhibitor of the oncogenic KRAS-G12C mutant that works through covalent binding to the C12 residue. (B) Cultures of human lung cancer H358 cells were nucleofected with SpCas9 protein and synthetic gRNA/tracrRNA targeting the 12C mutation in KRAS present in these cells along with an oligonucleotide comprising the mutation of interest (MUT), which was another oncogenic KRAS mutation (12D), and an oligonucleotide comprising a synonymous mutation (120*). At Day 2 after nucleofection, an aliquot of the cell culture was analysed by PCR amplification of the genomic target site and NGS of the PCR product to determine the ratio of MUT to 12C* alleles in the cell population. Thereafter, cells were split in two cultures, and cultured in the absence or presence of 120 nM AMG 510 until Day 12, whereafter the mutation analysis was repeated on each culture. Data represent ratios normalized to the value at Day 2.

FIG. 8 shows use of the method to test if mutations affect a given cell parameter such as DNA replication. (A) Cultures of human lung cancer NCI-H358 cells were nucleofected with Cas9 protein, synthetic gRNA/tracrRNA targeting and overlapping the 12C genomic site in KRAS-12C to be mutated along with an oligonucleotide comprising the mutation of interest (C12D) in KRAS-12C and an oligonucleotide comprising a synonymous mutation (12C* or MUT*). At Day 5 after nucleofection and culture in the presence of 120 nM of the 12C-directed inhibitor AMG 510, the cells were pulsed for 2 h with S-phase marker EdU and stained for EdU. Finally, cells were FACS isolated according to being either positive or negative for EdU. (B) Each population isolated in (A) was analysed by PCR amplification of the genomic target site and NGS of the PCR product to determine the ratio of KRAS-12D to KRAS-12C* alleles. Data represent ratios normalized to the value in the EdU positive population.

FIG. 9 shows use of the method to test if mutations affect a given cell parameter such as DNA damage. (A) Cultures of human breast epithelial MCF10A-BRCA2+/− cells expressing SpCas9 were transfected with synthetic gRNA/tracrRNA targeting a genomic site in BRCA2 overlapping the site to be mutated along with an oligonucleotide comprising the mutation of interest (MUT), which was a known pathogenic variant (T2722R), and an oligonucleotide comprising a synonymous mutation (WT*). At Day 4 after transfection, the cells were immunostained for a marker for DNA damage (γH2X), subjected to FACS analysis and finally, cells were FACS isolated according to either high levels of DNA damage (high γH2X) or low levels of DNA damage (low γH2X). (B) Each population isolated in (A) was analysed by PCR amplification of the genomic target site and NGS of the PCR product to determine the ratio of MUT to WT* alleles. Data represent ratios normalized to the value in the high γH2X population and are means+/−SD of 3 independent experiments. A two-tailed unpaired t-test was used to calculate the significance. *** indicates p<0.001.

FIG. 10 shows cultures of human breast epithelial MCF10A cells expressing SpCas9 that were transfected with synthetic gRNA/tracrRNA targeting a genomic site overlapping the site to be mutated along with an oligonucleotide comprising the mutation of interest (Q40STOP) in EGFR and an oligonucleotide comprising a synonymous mutation (WT*). At Day 3 after transfection, the cells were shifted to serum and growth factor-depleted medium with supplementation of neuregulin. On Day 8, the cells were seeded on a Matrigel-coated, cell-permeable membrane in the upper chamber of a Transwell invasion chamber in medium with no supplements, except for EGF as chemo-attractant in the lower chamber. At Day 9, the cells in the upper and lower chambers were analysed by PCR amplification of the genomic target site and NGS of the PCR product to determine the ratio of mutation of interest (MUT) to WT* in the two cell populations. Data represent ratios normalized to the value of the upper chamber.

FIG. 11 shows the principle of the method with emphasis on its use as a multiparametric functional analysis of genetic sequence variants by determining mutant:WT* ratios as a function of a temporal parameter or a spatial parameter that can take multiple forms. The figure is further described in the Examples.

FIG. 12 shows that the specific design of the PCR/NGS target site analysis of the method determines nature and absolute frequencies of all editing outcomes in the cell population, allowing critical controls. (A) Schematic representation of the target site PCR and amplicon NGS analysis. (B) An example of editing outcomes at the BRCA-T2722 target site in a cell population. The figure and results are further described in the Examples.

FIG. 13 shows that the method contains “built-in loss-of-heterozygosity” to reveal effects of loss-of-function mutations in tumor suppressor genes in diploid cells. (A) MCF10A cells (BRCA2+/+) expressing SpCas9 were transfected with synthetic gRNA/tracrRNA targeting a genomic site in BRCA2 overlapping the site to be mutated along with an oligonucleotide comprising the mutation of interest (MUT), which was a known pathogenic variant (T2722R), and an oligonucleotide comprising a synonymous mutation (WT*). At Day 2 after transfection, an aliquot of the cell culture was analysed by PCR amplification of the genomic target site and NGS of the PCR product to determine the ratio of 2722R to WT* alleles in the cell population. Thereafter, cells were cultured until Day 12, where after the mutation analysis was repeated. Data represent ratios normalized to the value at Day 2 and are means of 3 independent experiments +/−SD. A two-tailed paired t-test was used to calculate the significance. ** indicates p<0.01. (B) Editing outcomes at the BRCA2-T2722 target site in individual cells at Day 2. (C) A schematic illustration of the “loss-of-heterozygosity” feature inherent to the method. The figure and results are further described in the Examples.

FIG. 14 shows use of the method to determine mechanism of drug resistance and that drugs act on-target to inhibit cancer cells, demonstrated by the mutation V550M in the receptor gene FGFR4, which causes resistance to the selective FGFR4 inhibitor Fisogatinib, a drug for liver cancers overexpressing the growth factor FGF19. Data represent ratios normalized to the value at Day 2 and are means of 3 independent experiments +/−SD. Two-tailed paired t-tests were used to calculate the significance in all cases. NS=not significant. ** indicates p<0.01. The figure and results are further described in the Examples.

FIG. 15 shows the use of the method to determine mechanism of drug resistance demonstrated by the oncogenic mutation Y537S in the estrogen receptor gene ESR1, the major resistance mechanism to the estrogen antagonist Tamoxifen, the mainstay drug for estrogen receptor-positive breast cancers. Data represent ratios normalized to the value at Day 2 and are means of 3 independent experiments +/−SD. A two-tailed paired t-test was used to calculate the significance. ** indicates p<0.01. The figure and results are further described in the Examples.

FIG. 16 shows in vivo use of the method to determine mechanism of drug resistance and that drugs act on-target to inhibit tumor growth, demonstrated using the oncogenic KRAS-G12C mutant covalent inhibitor AMG 510 (for inhibitor, see FIG. 7). (A) A culture of human lung cancer H358 cells was nucleofected with SpCas9 protein and synthetic gRNA/tracrRNA targeting the oncogenic 12C mutation in KRAS present in these cells along with an oligonucleotide comprising the mutation of interest, which was another oncogenic KRAS mutation (12D), and an oligonucleotide comprising a synonymous mutation (120*). At Day 2 after transfection, an aliquot of the cell culture was analysed by PCR amplification of the genomic target site and NGS of the PCR product to determine the ratio of 12D versus 12C* alleles in the cell population. Next, the cells were xenografted into 2 nude mice and two weeks after, either mouse was treated once per day with vehicle (−) or AMG 510 (+). On day 57, the mutation analysis was repeated on the resultant tumors to determine the selection effect of AMG 510 on KRAS-12C* versus KRAS-12D variants in the tumors. (B) Determination of tumor volumes over time. (C) Representative mice photographed on day 40. Data represent ratios normalized to the value at Day 2 and are means of 3 independent experiments +/−SD. A two-tailed unpaired t-test was used to calculate the significance in (A), while a two-tailed paired t-test was used to calculate significance in (B). NS=not significant. ** indicates p<0.01. **** indicates p<0.0001. The figure and results are further described in the Examples.

FIG. 17 shows use of the method to test if a mutation affects a given cell parameter of interest such as DNA replication or apoptosis by determining mutant:WT* ratios in cell populations spatially separated by FACS according to markers for these parameters. (A) Analysis of proliferation. (B) Analysis of apoptosis. Data represent ratios normalized to the value in the (A) S-phase (EdU) negative population or (B) apoptosis (BrdU) negative population. Data are means of 3 independent experiments +/−SD. Two-tailed paired t-tests were used to calculate the significance in all cases. ** indicates p<0.01. The figure and results are further described in the Examples.

FIG. 18 shows use of the method to test if a mutation affects a given cell parameter of interest such as cell migration/invasion by determining mutant:WT* ratios in cell populations spatially separated by a motility/invasion barrier. (A) Schematic of the experiment. (B) Cells in the upper and lower chambers were analysed by PCR amplification of the genomic target site and NGS of the PCR product to determine the ratio of 1047R to WT* in the two cell populations. Data represent ratios normalized to the value of the upper chamber and are means of 3 independent experiments +/−SD. A two-tailed paired t-test was used to calculate the significance. * indicates p<0.1. The figure and results are further described in the Examples.

FIG. 19 shows the use of the method to test if a putative loss-of-function (i.e. candidate driver) mutation in a tumor suppressor gene increases cell proliferation/survival consistent with it being a bona-fide tumor suppressor gene. In this experiment, known pathogenic MLH1 variants were analyzed. Data represent ratios normalized to the value at Day 2 and are means of 3 independent experiments +/−SD. Two-tailed paired t-tests were used to calculate the significance in all cases. ** indicates p<0.01. The figure and results are further described in the Examples.

FIG. 20 shows the use of the method to categorize cancer-associated variants as pathogenic and to predict their PARP-inhibitor responsiveness, using the ATM gene as an example, which underlies hereditary breast and ovarian cancer. Data represent ratios normalized to the value at Day 2 and show a representative experiment. The figure and results are further described in the Examples.

FIG. 21 shows the use of the method to categorize cancer-associated variants as pathogenic, using the MLH1 gene as an example, which underlies hereditary colorectal cancer (Lynch syndrome). Data represent ratios normalized to the value at Day 2 and are means of 3 independent experiments +/−SD. Two-tailed paired t-test were used to calculate the significance in all cases. NS=not significant. ** indicates p<0.01. *** indicates p<0.001. The figure and results are further described in the Examples.

FIG. 22 shows that the method also works with NG-SpCas9. Cultures of human breast epithelial MCF10A cells expressing NG-SpCas9 were transfected with synthetic gRNA/tracrRNA targeting genomic sites in BRCA2 overlapping the sites to be mutated along with an oligonucleotide comprising the mutation of interest (MUT), which was as a known pathogenic variant (T2722R or I2627N), and an oligonucleotide comprising a corresponding synonymous mutation (WT*). Data represent ratios normalized to the value at Day 2 and are means of 3 technical replicates+/−SD. Two-tailed paired t-tests were used to calculate the significance in all cases. ** indicates p<0.01. The figure and results are further described in the Examples.

FIG. 23 shows cultures of human breast epithelial MCF10A-BRCA2+/− cells expressing SpCas9 that were transfected with synthetic gRNA/tracrRNA targeting genomic sites in BRCA2 overlapping the sites to be mutated along with an oligonucleotide comprising the mutation of interest (MUT), which was as either a known benign variant (N289H) or a variant of unknown significance (D946V), and an oligonucleotide comprising a synonymous mutation (WT*). In addition to cells with knockin of the mutation of interest or the WT* synonymous mutation, a substantial fraction of cells will have introduction of a frameshifting indel (insertion/deletion) mutation, which are loss-of-function mutations (knockout=KO). The fate of these cells over time can also be analysed in the same cell culture, where the neutral variant was investigated. At Days 2 and 12 after transfection, an aliquot of each culture was analysed by PCR amplification of the genomic target site and NGS of the PCR product to determine the ratio of frameshifting indel knockout (KO) mutations to WT* alleles in the cell population. Data are means of 3 independent experiments +/−SD. A two-tailed paired t-test was used to calculate the significance.** indicates p<0.01. * indicates p<0.1.

### DETAILED DESCRIPTION

### Definitions

Binding region of a nuclease: the term refers to the region or portion of a target nucleic acid sequence to which a given nuclease actually binds. The CRISPR-Cas binding region comprises an approximately 20 nucleotides long sequence that binds the crRNA by Watson-Crick base pairing. In addition, the Cas protein binds a 2-5 nucleotides long PAM sequence located on the opposite DNA strand and immediately 3′ to the crRNA binding region. The TALEN binding region comprises two 12-16 nucleotides long sequences separated by 5-15 base pairs and located on opposing strands of DNA and that each bind one TALEN of the TALEN dimer. The length of the binding region depends on the number of TALE domains in the individual TALEN monomers. The ZFN binding region comprises two 9-15 nucleotides long sequences separated by 4-6 base pairs and located on opposing strands of DNA and that each bind one ZFN of the ZFN dimer. The length of the binding region depends on the number of zinc finger domains in the individual ZFN monomers.

Cell marker: the term refers to any marker that can be used to characterise a given population or subpopulation of cells, in particular a parameter of interest.

Coding region or coding sequence (CDS): these terms, used herein interchangeably, refer to a portion of DNA or RNA, excluding introns, which results in a protein upon translation.

CRISPR/Cas nuclease: the term refers to members of the CRISPR-Cas family. The prokaryotic adaptive immune system CRISPR-Cas (clustered regularly interspaced short palindromic repeats and CRISPR-associated proteins) can bind and cleave a target DNA sequence through RNA-guided recognition. According to their molecular architecture, the different members of the CRISPR-Cas system have been classified in two classes: class 1 encompasses several effector proteins, whereas class 2 systems use a single element. The CRISPR-associated proteins (Cas) known to date include Cas9, Cas12a (formerly Cpf1) and Cas13 (formerly C2c2).

Difference between an initial and a subsequent ratio: the term refers to subtracting the value of the initially measured ratio, from the value of the subsequently measured ratio.

For example, if an initial ratio of 1 is measured, and a subsequent ratio of 5 is measured, the difference between the initial and the subsequent ratio is 5-1=4, and therefore positive. In a similar manner, if an initial ratio of 1 is measured, and a subsequent ratio of 0.5 is measured, the difference between the initial and the subsequent ratio is 0.5-1=−0.5, and therefore negative.

Frameshift indel: the term refers to a genetic mutation caused by indels (insertions or deletions) of a number of nucleotides in a DNA sequence that is not divisible by three. This results in a frameshift in the open reading frame of the encoded gene. A change in the frame of the open reading frame often leads to a change in the encoded chain of amino acids or to the introduction of premature stop codons resulting in a shortened and/or non-functional protein product encoded by that gene.

Genotype: the term as used herein refers to an organism comprising a specific set of genes. Thus, two organisms comprising identical genomes are of the same genotype. An organism's genotype in relation to a particular gene is determined by the alleles carried by said organism. In diploid organisms the genotype for a given gene may be AA (homozygous, dominant) or Aa (heterozygous) or aa (homozygous, recessive).

Guide RNA: the term will herein be used interchangeably with “crRNA” and refers to the RNA molecule, which is required for recognition of a target nucleic acid sequence by CRISPR-Cas proteins.

Homologue: a homologue or functional homologue may be any polypeptide that exhibits at least some sequence identity with a reference polypeptide and has retained at least one aspect of the original functionality.

Indel mutation: the term refers to a mutation occurring in a nucleic acid sequence, for example a genomic sequence of an organism, including non-coding regions, coding regions and regulatory sequences such as promoters, which results in insertion and/or deletion of a number of base pairs.

Meganuclease: the term refers to an endodeoxyribonuclease characterized by a large recognition site (double-stranded DNA sequences of 12 to 40 base pairs). Meganucleases can be modified to recognise a given target nucleic acid.

Mixed population of cells: the term herein refers to the entire cell population which is contacted with the nuclease and/or the targeting means, and in which it is desired to generate a double-strand break and/or a single-strand break at least in some of the cells. The mixed population of cells therefore typically contains the following types of cells: cells in which no break has been generated and cells in which a break has been generated, and in which either i) perfect repair has occurred and no mutations have been introduced in the target region, ii) an indel mutation has been introduced in the target region, iii) a first oligonucleotide comprising a mutation of interest, preferably a non-silent mutation, preferably a non-synonymous mutation, wherein said mutation preferably lies within the binding region of the nuclease and otherwise identical to, or complementary to the target nucleic acid sequence has been integrated, or copied, or ii) a second oligonucleotide comprising a synonymous mutation, and wherein said synonymous mutation is located in the same position, or close to the position of said mutation of interest, such as within 10 nucleotides of the position of said mutation of interest, and wherein said synonymous mutation preferably lies within the binding region of the nuclease, has been integrated, or copied.

Mutation: the term refers to a mutation occurring in a nucleic acid sequence, for example a genomic sequence, including non-coding regions, coding regions and regulatory sequences such as promoters. Throughout this disclosure, the term “mutation” refers to a change in nucleic acid sequence compared to a reference sequence, for example a wild-type sequence. A mutation may result in a change in the encoded amino acid sequence (non-synonymous mutation) or it may result in no change in the encoded amino acid sequence (synonymous mutation) compared to the amino acid sequence encoded by the reference or wild-type sequence.

Non-silent mutation: the term refers to a mutation occurring in a nucleic acid sequence, for example a genomic sequence of an organism, which results in an observable change in the organism's phenotype or properties. Often a non-silent mutation results in a change of amino acid sequence, efficiency of translation, splicing, a frameshift or a change in a regulatory region, such as a promoter. A non-silent mutation may thus be any mutation in a non-coding region of a genome, or it may be a mutation in a coding region of a genome.

Non-synonymous mutation: the term as used herein refers to a mutation occurring in a nucleic acid sequence, for example a genomic sequence of an organism, which results in a change in the encoded amino acid sequence compared to the amino acid sequence encoded by a reference sequence, typically a wild-type sequence. A non-synonymous mutation thus occurs in coding regions and may be any mutation in a coding region of a genome which changes the amino acid sequence of the translation product, e.g. by encoding a different amino acid (e.g. missense mutations, read-through mutations), by introduction of a premature stop codon (nonsense mutations) and/or by introduction of a frameshift.

Nuclease: the term refers to an enzyme capable of cleaving the phosphodiester bonds between nucleotides of nucleic acids. Nucleases variously effect single and double stranded breaks in their target nucleic acids. The term encompasses both endonucleases, which effect the break in the nucleic acids from within, and exonucleases, which effect the break from the terminal end(s) of the nucleic acids. The term encompasses deoxyribonucleases acting on DNA, and ribonucleases acting on RNA. Throughout the present disclosure it will be understood that the nucleases can be provided to a cell either as part of a polynucleotide, e.g. a DNA molecule or an RNA molecule, or directly as protein.

Parameter of interest: the term refers to a parameter which is measurable, and which is of interest. In particular, parameters of interest herein refer to parameters associated with the cells that are being investigated, and comprises spatial parameters and temporal parameters, which are further defined below. Examples of relevant parameters of interest include: cell proliferation, cell growth, fitness, cell motility, cell invasiveness, cellular metabolism, cell differentiation, DNA damage, expression levels of pre-defined genes or proteins, resistance to a compound, sensitivity to a compound, production of a compound, anoikis, senescence, apoptosis, DNA methylation, anchorage-independent cell growth, contribution to tumor growth, and protein post-translational modification. For the purpose of the present disclosure, the parameter of interest may be a temporal parameter or a spatial parameter, or a combination of both.

PCR reagents: the term as used herein refers to reagents, which are added to a PCR in addition to a sample and a set of primers. The PCR reagents comprise at least nucleotides and a nucleic acid polymerase. In addition, the PCR reagents may comprise other compounds such as salt(s) and buffer(s).

PCR: the term as used herein refers to a polymerase chain reaction. A PCR is a reaction for amplification of nucleic acids. The method relies on thermal cycling, and consists of cycles of repeated heating and cooling of the reaction to obtain sequential melting and enzymatic replication of said DNA. In the first step, the two strands forming the DNA double helix are physically separated at a high temperature in a process also known as DNA melting. In the second step, the temperature is lowered allowing enzymatic replication of DNA. PCR may also involve incubation at additional temperature in order to enhance annealing of primers and/or to optimize the temperature(s) for replication. In a PCR, the temperature generally cycles between the various temperatures for a number of cycles.

Phenotype: the term as used herein is the composite of the organism's, including single cells' observable characteristics or traits.

Protospacer adjacent motif (PAM): the term refers to the DNA sequence immediately downstream of (3′ to) the DNA sequence targeted by the crRNA of a CRISPR-Cas system and located on the opposite strand. The crRNA of a crRNA-Cas complex is capable of recognizing and hybridizing to a target DNA sequence only if it comprises a PAM, which binds the Cas protein.

Set of primers flanking a target sequence or nucleic acid: the term as used herein refers to a set of two primers flanking a target sequence or a target nucleic acid, so that one primer comprises a sequence identical to a region located 5′ to the target sequence and preferably 5′ to the region homologous to the oligonucleotide comprising the mutation (also referred to as “forward primer”) and one primer comprises a sequence identical to a region located on the opposite DNA strand 3′ to the target sequence and preferably 3′ to the region homologous to the oligonucleotide comprising the mutation (also referred to as “reverse primer”). The “set of primers” can amplify the target sequence when added to a PCR together with a nucleic acid comprising the target sequence region and PCR reagents under conditions allowing amplification of said target sequence.

Silent mutation: the term refers to a mutation occurring in a nucleic acid sequence, for example a genomic sequence of an organism, which does not result in an observable effect on the organism's phenotype or properties. Often a silent mutation is a mutation that does not result in a change of amino acid sequence. A silent mutation may thus be any mutation in a non-coding region of a genome, or it may be a mutation in a coding region of a genome, which does not change the amino acid sequence of the translation product or interfere with translation in other ways.

Synonymous mutation: the term refers to a mutation occurring in a nucleic acid sequence, for example a genomic sequence of an organism, which results in no change in the encoded amino acid sequence. The mutation, unless indicated otherwise, is synonymous compared to the reference sequence, typically a wild-type sequence. A synonymous mutation may thus be any mutation in a coding region of a genome, which does not change the amino acid sequence of the translation product.

Spatial parameter: the term refers to a parameter, in particular a parameter of interest, which is measured and/or monitored spatially in a defined cell population, which may be a total cell population, or a subpopulation of cells. The spatial parameter can, but does not need to, be measured at different time points. The spatial parameter typically reflects a property of the cell population or subpopulation, such as size, biomarker expression, motility, or any parameter that can be measured at a given time point and reflects a spatial or physical property of the cell population or subpopulation. A spatial parameter can thus be measured and/or monitored for a subpopulation of cells resulting for example from FACS sorting, or from isolation using specific antibodies linked to a matrix or for an entire population of cells in e.g. a physical compartment, e.g. an organ or a sub-region of an organ, a tumor or a sub-region of a tumor or on one side of a membrane. Examples of relevant spatial parameters of interest include: cell proliferation, cell growth, fitness, cell motility, cell invasiveness, cellular metabolism, cell differentiation, DNA damage, expression levels of pre-defined genes or proteins, resistance to a compound, sensitivity to a compound, production of a compound, anoikis, senescence, apoptosis, DNA methylation, and protein post-translational modification. The value of the spatial parameter obtained for a given population or subpopulation may in some instances be compared to pre-determined threshold values or to the values for the same parameter obtained in a reference population, which could be another subpopulation or the whole population. However it will be clear from the below description that the value of the spatial parameter for the given population or subpopulation can also be informative on its own, i.e. without comparison to a pre-determined threshold value or the value for a reference population.

Subpopulation of cells: the term refers to a fraction of the mixed population of cells, which comprise at least one single cell. The subpopulations may be physically defined and separated, for example one subpopulation can refer to the cells comprised within a given tumor or sub-region of a tumor or an organ or a sub-region of an organ, while another subpopulation can refer to the cells comprised in healthy tissue or in another organ. As another example, one subpopulation can refer to cells on one side of a cell permeable membrane/matrix, while another subpopulation can refer to cells on the other side. In other instances, the subpopulations are defined on the basis of a value of a spatial or temporal parameter of interest, for example a first subpopulation comprises cells having high values and a second subpopulation comprises cells having low values for the parameter of interest, and such subpopulations may be isolated/separated by FACS. In yet other instances, the subpopulations may be defined by expression of a cell surface protein or epitope, and such subpopulations may be isolated by antibodies linked to a matrix or similar. More than two subpopulations may be defined from a mixed population of cells.

Target nucleic acid: the term will be used interchangeably with the term “target sequence” and herein refers to any nucleic acid sequence within which it is desirable to generate a single-stranded or double-stranded break by the action of a nuclease, for example to introduce a mutation such as a non-silent mutation, a non-synonymous mutation, a silent mutation or a synonymous mutation. Furthermore, the target sequence is preferably a nucleic acid sequence, which can be amplified by PCR technology using primers flanking the target sequence.

Targeting means: the term refers to a moiety or a molecule, which enables a nuclease to recognise its target nucleic acid. For a CRISPR/Cas nuclease, the targeting means refers to the guide RNA or crRNA. For TALENs, the targeting means refers to the TAL effector DNA-binding domains. For ZFNs, the targeting means refers to the zinc finger DNA-binding domains. The targeting means may thus be a moiety of the nuclease (for ZFNs and TALENs), or it may be a different molecule altogether (for CRISPR/Cas systems).

Targeting: the term “targeting” as understood herein refers to the ability of a molecule to identify a nucleotide sequence. For example, an enzyme or a DNA binding domain or molecule may recognise a nucleic acid sequence as a potential substrate and bind to it. Preferably, the targeting is specific.

Temporal parameter: the term refers to a parameter, in particular a parameter of interest, which is measured and/or monitored over time, i.e. the temporal parameter is determined for at least two different time points: an initial time point and a subsequent time point. The ratio of the values measured for the temporal parameter at the initial time point and at the subsequent time point can be calculated to determine a change in ratio. A positive change in ratio for the temporal parameter means that the subsequent value is greater than the initial value, while a negative change in ratio for the temporal parameter means that the subsequent value is smaller than the initial value; no change in ratio indicates that there is no change in the temporal parameter. Examples of relevant temporal parameters of interest include: allele frequencies in a population, cell growth, anchorage-independent cell growth, contribution to tumor growth, fitness, cell motility, cell invasiveness, cellular metabolism, DNA damage, expression levels of pre-defined genes and/or proteins, resistance to a compound, sensitivity to a compound, production of a compound, anoikis, senescence, contact inhibition and apoptosis. The temporal parameter can be measured for an entire population of cells, or for a given subpopulation of cells.

Transcription activator-like effector nuclease (TALEN): these nucleases can be engineered to introduce a break in a target nucleic acid sequence. They are generated by fusing TAL effector DNA-binding domains to a DNA cleavage domain. Engineering of the transcription activator-like effector (TALE) moieties of the TALEN can be performed so that it recognises any given target nucleic acid sequence, which is then cleaved by the DNA cleavage domain. TALENs thus consist of a DNA-binding domain and a DNA cleavage domain, and can introduce a double-strand break in the target nucleic acid. TALENs function as heterodimers composed of two distinct TALENs that bind target nucleic acid sequences on opposing DNA strands.

Zinc finger nuclease: the term refers to enzymes generated by fusing zinc finger DNA-binding domains to a DNA-cleavage domain. Engineering of the zinc finger moieties of the ZFN can be performed so that it recognises any given target nucleic acid sequence, which is then cleaved by the DNA cleavage domain. ZFNs thus consist of a DNA cleavage domain and a DNA-binding domain, and can introduce a double-stranded break in their target nucleic acid. ZFNs function as heterodimers composed of two distinct ZFNs that bind target nucleic acid sequences on opposing DNA strands.

Methods for Assessing the Effects of a Mutation of Interest in a Cell

The present disclosure relates to accurate, fast, simple, cost-efficient methods for assessing the effects of a mutation of interest in a cell. The methods as disclosed herein can be used to determine how genetic variants impact cell proliferation, survival, motility, metabolism, differentiation and/or other cell parameters per se or in response to any environmental stimulus of interest, e.g. drugs, stresses, pathogens and/or nutrients. The methods are scalable and work in any species.

In short, the present methods rely on the introduction, in at least some cells of a cell population, of either a first oligonucleotide comprising a mutation of interest, or a second oligonucleotide comprising a synonymous mutation, where the synonymous mutation does not introduce a change in the encoded amino acid sequence relative to the amino acid sequence encoded by the target nucleic acid sequence or wild-type nucleic acid sequence. Upon generation of a single-stranded or double-stranded break by a nuclease in a target nucleic acid sequence, either the first or the second oligonucleotide are introduced in, or copied into the target nucleic acid sequence. There will also be cells in which the break is perfectly repaired or in which indel mutagenesis occurs, of which the first events will be neutral and the latter events will normally not interfere with the method, but rather will be beneficial for the method in some cases. The population in which the synonymous mutation of the second oligonucleotide is introduced serves as an internal control, which facilitates determining the effect of the mutation of interest introduced via the first oligonucleotide. The resulting population, comprising cells without modification of the target nucleic acid sequence, cells in which an indel mutation has been introduced in the target nucleic acid sequence, cells in which the mutation of interest has been introduced in the target nucleic acid sequence and cells in which a synonymous mutation has been introduced in the target nucleic acid sequence, can then be analysed, and the effects of the mutation of interest assessed as described in detail below. The present methods greatly facilitate assessing the effect of a mutation of interest, and eliminate the need for multiple steps, thereby also reducing the time needed to assess said effect.

In one aspect, the present invention provides a method for assessing the effects of a mutation of interest in a cell, said method comprising the steps of:
i) providing a cell population comprising a target nucleic acid sequence;
ii) introducing in at least some of the cells of the cell population:
   a) a nuclease or a polynucleotide encoding said nuclease, wherein the nuclease is capable of generating one or more single-strand breaks (SSBs) or double-strand breaks (DSBs) in a target nucleic acid sequence, and targeting means directing the nuclease to the target nucleic acid sequence, whereby the nuclease is capable of binding to a binding region of the target nucleic acid sequence, and whereby the nuclease is capable of generating one or more single-stranded breaks (SSBs) or double-strand breaks (DSBs) in the target nucleic acid sequence;
   b) a first oligonucleotide comprising a mutation of interest, preferably a non-silent mutation, preferably a non-synonymous mutation, wherein said non-synonymous mutation introduces a change in the encoded amino acid sequence compared to the amino acid sequence encoded by the target nucleic acid sequence, and otherwise identical to or complementary to said target nucleic acid sequence, wherein said mutation of interest preferably lies within the binding region of said nuclease; and
   c) a second oligonucleotide comprising a synonymous mutation, wherein said synonymous mutation preferably lies within the binding region of said nuclease, and wherein said synonymous mutation introduces no change in the encoded amino acid sequence compared to the amino acid sequence encoded by the target nucleic acid sequence;
   whereby said first oligonucleotide or said second oligonucleotide is integrated in, or copied into, the target nucleic acid sequence of at least some of the cells, thereby obtaining a mixed population of cells comprising cells in which the mutation of the first or the second oligonucleotides has not been introduced, cells in which only the mutation of the first oligonucleotide has been introduced, and cells in which only the mutation of the second oligonucleotide has been introduced;
iii) incubating the mixed population of cells in a medium for a determined duration, under conditions allowing a parameter of interest to be monitored, wherein the parameter of interest is a temporal parameter and/or a spatial parameter;
iv) determining the effect of the mutation of interest on the parameter of interest, wherein:
   A. if the parameter of interest is a temporal parameter, determining the effect of the mutation of interest on the parameter of interest comprises the steps of:
      v) determining an initial ratio of cells in which the mutation of interest has been introduced in the target nucleic acid sequence to cells in which the synonymous mutation has been introduced in the target nucleic acid sequence, wherein the initial ratio of cells is determined at an initial time point; determining a subsequent ratio of cells in which the mutation of interest has been introduced in the target nucleic acid sequence to cells in which the synonymous mutation has been introduced in the target nucleic acid sequence, wherein the subsequent ratio of cells is determined at a subsequent time point; and determining a change in ratio between the initial ratio and the subsequent ratio; and
      vi) correlating said change in ratio to the parameter of interest, and/or
   B. if the parameter of interest is a spatial parameter, determining the effect of the mutation of interest on the parameter of interest comprises the steps of:
      v) defining and/or spatially separating subpopulations of cells on the basis of said spatial parameter of interest in each subpopulation, preferably wherein the spatial parameter of interest is different in each subpopulation;
      vi) determining, for each subpopulation, a ratio of cells in which the mutation of interest has been introduced in the target nucleic acid sequence to cells in which the synonymous mutation has been introduced in the target nucleic acid sequence; and
      vii) correlating said ratio to the measured spatial parameter of interest for each subpopulation;
         thereby assessing the effect of the mutation on the parameter of interest.

The nuclease, the first and/or second oligonucleotide may be introduced, or copied into at least some of the cells in the cell population by any method known in the art. In some embodiments, the nuclease, the first and/or second oligonucleotide are introduced into the cells by electroporation. In some embodiments, the nuclease, the first and/or second oligonucleotide are introduced into the cells by heat shock. In some embodiments, the nuclease, the first and/or second oligonucleotide are introduced into the cells by liposome transfection (lipofection). In some embodiments, the nuclease, the first and/or second oligonucleotide are introduced into the cells by viral delivery. In yet other embodiments, the nuclease, the first and/or second oligonucleotide are introduced into the cells by a non-liposomal transfection reagent such as a FuGENE transfection reagent. In some embodiments, the nuclease may be expressed in the cells and only crRNA and first and/or second oligonucleotide is introduced via any of above-mentioned methods.

In some embodiments, the determined duration is at least 4 hours, such as at least 8 hours, such as at least 12 hours, such as at least 18 hours, such as at least 24 hours, such as at least 48 hours, such as at least 72 hours.

It may also be desirable to wait for longer periods, e.g. if the mutation of interest first has an effect after selective pressure has been applied. Thus, in some embodiments, the determined duration is at least such as at least 1 week, such as at least 2 weeks, such as at least 3 weeks, such as at least 4 weeks, such as at least 2 months, such as at least 4 months, such as at least 6 months, such as at least 8 months, such as at least 10 months, such as at least 12 months, such as at least 1½ year, such as at least 2 years.

In some embodiments, the time-to-result (between steps iii and iv) is at the most 3 weeks, such as at the most 2 weeks, such as at the most 12 days, such as at the most 10 days, such as at the most 8 days, such as at the most one week, such as at the most 6 days, such as at the most 5 days, such as at the most 4 days, such as at the most 3 days, such as at the most 2 days, such as at the most 1 day, such as at the most 12 hours, such as at the most 6 hours.

In some embodiments, the time-to-result (between steps iii and iv) is at the most 2 years, such as at the most 1½ years, such as at the most 12 months, such as at the most 10 months, such as at the most 8 months, such as at the most 6 months, such as at the most 4 months, such as at the most 2 months, such as at the most 4 weeks.

In some embodiments, the time between the initial time point and the subsequent time point is at least 4 hours. In some embodiments, the time between the initial time point and the subsequent time point is at least 8 hours. In some embodiments, the time between the initial time point and the subsequent time point is at least 12 hours. In some embodiments, the time between the initial time point and the subsequent time point is at least 18 hours. In some embodiments, the time between the initial time point and the subsequent time point is at least 24 hours. In some embodiments, the time between the initial time point and the subsequent time point is at least 48 hours. In some embodiments, the time between the initial time point and the subsequent time point is at least 72 hours.

In some embodiments, the time between the initial time point and the subsequent time point is at least 1 week. In some embodiments, the time between the initial time point and the subsequent time point is at least 2 weeks. In some embodiments, the time between the initial time point and the subsequent time point is at least 3 weeks. In some embodiments, the time between the initial time point and the subsequent time point is at least 4 weeks. In some embodiments, the time between the initial time point and the subsequent time point is at least 2 months. In some embodiments, the time between the initial time point and the subsequent time point is at least 4 months. In some embodiments, the time between the initial time point and the subsequent time point is at least 6 months. In some embodiments, the time between the initial time point and the subsequent time point is at least 8 months. In some embodiments, the time between the initial time point and the subsequent time point is at least 10 months. In some embodiments, the time between the initial time point and the subsequent time point is at least 12 months. In some embodiments, the time between the initial time point and the subsequent time point is at least 1½ years. In some embodiments, the time between the initial time point and the subsequent time point is at least 2 years.

In some embodiments, steps iii) and iv) of the method as described herein, and optionally steps A.v) to A.vi) and/or steps B.v) to B.vii), are performed more than once for further predetermined duration(s).

In some embodiments, the target nucleic acid sequence is within a gene, a promoter or an enhancer of a gene, wherein the gene is or is suspected to be an oncogene. In some embodiments, said gene is or is suspected to be a proto-oncogene. In some embodiments, said gene is or is suspected to be a tumor suppressor gene. In some embodiments, said gene is or is suspected to be a gene encoding an enzyme such as an enzyme involved in the production of a compound such as a metabolite, a resistance gene, such as a gene involved in resistance to a compound, a pharmaceutical compound, or a pathogen such as a virus. In some embodiments, said gene is or is suspected to be a gene encoding a protein involved in cellular fitness and/or growth. In some embodiments, said gene is or is suspected to be a gene encoding a protein for any cell function, a microRNA or a long non-coding RNA. The target nucleic acid may be genomic, i.e. comprised in the genome of the cells comprised within the cell population, or it may be extrachromosomal, e.g. on a vector or plasmid or may be comprised in DNA of an infected pathogen or invading organism within another cell.

In some embodiments, step A.v) and/or step B.vi) of the methods as disclosed herein comprises amplifying a region comprising the target nucleic acid sequence, such as by PCR, to produce an amplicon comprising the target nucleic acid sequence, optionally followed by sequencing, such as next-generation sequencing, of said amplicon, in order to determine the ratios of mutation of interest to synonymous mutation.

One advantage of the present methods is that only one set of primers is needed for analysing all types of cells in the mixed cell population. Another advantage is that if the primers are chosen so that they anneal outside the region substantially identical or complementary to the first or second oligonucleotides encompassing the mutations, the same primer set can be used to amplify cells having integrated, or copied the first oligonucleotide, cells having integrated, or copied the second oligonucleotide or cells having integrated or copied none of the oligonucleotides, and instead having performed error-free repair or having obtained an indel mutation at the target region. This critically allows the calculation of the absolute frequencies of introduction of the mutation of interest and the synonymous mutation in the cell population, which in turn allows determination of if the obtained results are statistically significant. This critically also allows for detection of frameshifting indel mutations such that they can serve as internal controls for mutations of interest that appear neutral, by being able to show that the method functioned properly. Thus, similar to the mutation of interest, the ratio of frameshifting indels to WT* can be determined according to a temporal or a spatial parameter to determine if the frameshifting (i.e. knockout/loss-of-function) indel mutation affects the parameter of interest.

Thus, in some embodiments, the mixed population also comprises cells in which the mutation of the first or the second oligonucleotides has been introduced, and/or wherein one or more indel mutations have been introduced in the target nucleic acid sequence.

In some embodiments, step A.v) further comprises a step of determining an initial frequency of cells with an indel in the target nucleic acid sequence which is different from the mutation of the first and the second oligonucleotides, wherein the initial frequency of cells is determined at said initial time point, and determining a subsequent frequency of cells with an indel in the target nucleic acid sequence which is different from the mutation of the first and the second oligonucleotides, wherein the subsequent frequency of cells is determined at said subsequent time point. Said initial frequency of cells with an indel in the target nucleic acid sequence may be determined at the initial time point and/or the subsequent frequency of cells at the subsequent time point.

In some embodiments, step B.vi) further comprises a step of, for each subpopulation, determining a frequency of cells with an indel in the target nucleic acid sequence which is different from the mutation of the first and the second oligonucleotides.

In some embodiments, the step of determining the ratio of cells of step A.v) is performed for only a fraction of the cell population. In some embodiments, the step of determining the ratio of cells of step B.vi) is performed for only a fraction of each subpopulation.

In some embodiments, the initial and the subsequent frequency of cells with an indel is further subdivided into, respectively, an initial and a subsequent frequency of cells with an indel resulting in a frameshift mutation and an initial and a subsequent frequency of cells with an indel not resulting in a frameshift mutation, wherein a subsequent frequency of cells with an indel resulting in frameshift mutation lower or higher than the initial frequency of cells with an indel resulting in frameshift mutation indicates that the frameshift indels are affecting the parameter of interest.

In some embodiments, the frequency of cells with an indel is further subdivided into a frequency of cells with an indel resulting in a frameshift mutation and a frequency of cells with an indel not resulting in a frameshift mutation, wherein a frequency of cells with an indel resulting in frameshift mutation in one subpopulation is substantially different from the frequency of cells with an indel resulting in frameshift mutations in a second subpopulation indicates that the frameshift indels are affecting the cells.

As determining the initial and subsequent ratios of cells in which the mutation of interest has been introduced in the target nucleic acid sequence to cells in which the synonymous mutation has been introduced in the target nucleic acid sequence may be best performed on the same population, it may be desirable to only determine said initial and subsequent ratios on a subset of the population, which serves as a surrogate for the ratio in the complete cell population.

Thus, in some embodiments, the step of determining a ratio of cells in which the mutation of interest has been introduced in the target nucleic acid sequence to cells in which the synonymous mutation has been introduced in the target nucleic acid sequence of step A.v) and/or step B.vi) is performed for only a fraction of the cell population and/or only a fraction of each subpopulation.

In some embodiments, the cell is a vertebrate cell, an invertebrate cell, a plant cell, a yeast cell, a fungal cell or a bacterial cell. In some embodiments, the cell may be part of a library, such as a yeast or a bacterial library, e.g. the Yeast GFP Clone Collection or the E. coli Keio Knockout Collection. These libraries may be used to screen the effect of mutations of interest on e.g. changes in location of specific protein followed by fluorescence microscopy, or e.g. whether the mutations have a larger or a smaller effect depending on if certain proteins are knocked out.

In some embodiments, the cell is a human cell. In some embodiments, the human cell is a primary cell, such as a cell isolated from a patient. Said cell may be isolated from a tumor, e.g. wherein a mutation-of-interest may be introduced in the cell to revert a target sequence, containing a putative cancer-driving mutation, to a wild type sequence in order to diagnose the putative cancer-driving mutation as either contributing to the cancer or not. In some embodiments, the human cell may be isolated from a patient with another genetic disease.

In some embodiments, the methods as described herein are performed in vivo. In yet other embodiments, the methods as described herein are performed in vitro.

Oligonucleotides Useful for the Current Methods

In some embodiments, the first oligonucleotide comprises at least one mutation of interest, such as 1, 2, 3, 4, 5 or more mutations of interest, within, or close to, the binding region of the nuclease, and the second oligonucleotide comprises at least one synonymous mutation, such as 1, 2, 3, 4, 5 or more synonymous mutations, within, or close to, the binding region of the nuclease. In other words, in some embodiments the first and second oligonucleotides differ only in the nature of the mutations they can introduce, but have otherwise the same characteristics, for example the same length and/or GC content.

In some embodiments, the first oligonucleotide comprises at least one mutation of interest, such as 1, 2, 3, 4, 5 or more mutations of interest, within the binding region of the nuclease, and the second oligonucleotide comprises two or more oligonucleotides that each comprise at least one synonymous mutation, such as 1, 2, 3, 4, 5 or more synonymous mutations, within the binding region of the nuclease.

In some embodiments, the first oligonucleotide comprises at least one first mutation, which is a synonymous mutation lying inside the binding region of the nuclease, and further comprises at least one second mutation, which is a non-synonymous mutation of interest lying outside the binding region of the nuclease, and wherein the second oligonucleotide comprises at least one synonymous mutation lying within the same region as the first mutation and further comprises at least one further synonymous mutation lying inside the same region as the second mutation, wherein the first mutation and the synonymous mutation when introduced in the target nucleic acid sequence prevent the nuclease and the targeting means from binding to and/or generating a further SSB or a further DSB in the resulting nucleic acid sequence. In some preferred embodiments, the first mutation and the synonymous mutation are identical.

In some embodiments, the first oligonucleotide and the second oligonucleotide differ only in the location of the mutation of interest. Thus, in some embodiments, the synonymous mutation of said second oligonucleotide is located in the same genomic position as the mutation of interest in said first oligonucleotide.

In some embodiments, said first oligonucleotide consists of or comprises a stretch of nucleotides identical to said second oligonucleotide except for said mutation of interest or wherein said second oligonucleotide consists of or comprises a stretch of nucleotides identical to said first oligonucleotide except for said synonymous mutation.

In some embodiments, the synonymous mutation of said second oligonucleotide is located in a different genomic position from the mutation of interest in said first oligonucleotide. In some embodiments, the synonymous mutation of said second oligonucleotide is located within 20 nucleotides, such as within 19 nucleotides, such as within 18 nucleotides, such as within 17 nucleotides, such as within 16 nucleotides, such as within 15 nucleotides, such as within 14 nucleotides, such as within 13 nucleotides, such as within 12 nucleotides, such as within 11 nucleotides, such as within 10 nucleotides, such as within 9 nucleotides, such as within 8 nucleotides, such as within 7 nucleotides, such as within 6 nucleotides, such as within 5 nucleotides, such as within 4 nucleotides, such as within 3 nucleotides, such as within 2 nucleotides, such as within 1 nucleotide from the genomic position of the mutation of interest in said first oligonucleotide.

In some embodiments, the first and the second oligonucleotides are of different lengths. In some embodiments, the first and the second oligonucleotides are of the same length.

In some embodiments, the synonymous mutation is a single base change compared to the genomic target region. In some embodiments, the mutation of interest is a single base change compared to the genomic target region.

The methods as described herein may also be used for assessing the effects of several different mutations of interest in a cell at the same time. Thus, the present methods may be used for multiplex assays, wherein the effects of multiple mutations are assessed simultaneously in a single experiment.

In some embodiments, the first oligonucleotide is a plurality of first oligonucleotides each comprising a pre-determined mutation of interest and the second oligonucleotide is a plurality of second oligonucleotides each comprising a pre-determined synonymous mutation.

In some embodiments, the first oligonucleotide comprises a plurality of pre-determined mutations of interest and the second oligonucleotide comprises a plurality of pre-determined synonymous mutations. In some embodiments, said second oligonucleotides are otherwise identical to said first oligonucleotides.

In some embodiments, the plurality of mutations is at least 2 different mutations, such as at least 3 different mutations, such as at least 4 different mutations, such as at least different mutations, such as at least 6 different mutations, such as at least 7 different mutations, such as at least 8 different mutations, such as at least 9 different mutations, such as at least 10 different mutations, such as at least 15 different mutations, such as at least 20 different mutations, such as at least 25 different mutations, such as at least different mutations, such as at least 35 different mutations, such as at least 40 different mutations, such as at least 45 different mutations, such as at least 50 different mutations or more.

In some embodiments, one or more of the first oligonucleotide, the second oligonucleotide, the targeting means and the polynucleotide encoding the nuclease are comprised within one or more vectors or plasmids, or within a virus.

In some embodiments, the first and/or the second oligonucleotide is part of the guide RNA capable of hybridizing to the genomic target region. This may be particularly useful if using prime editors to introduce the mutations of the first and/or the second oligonucleotide into cells of the cell population.

Prime editors are fusion proteins between a Cas9 nickase domain and an engineered reverse transcriptase domain. The prime editor may be a fusion of a Streptococcus pyogenes Cas9 nickase domain and a Moloney Murine Leukemia Virus reverse transcriptase domain, such as PE2 (SEQ ID NO: 9) described in Anzalone et al., 2019.

The prime editor protein is targeted to the editing site by a guide RNA, which not only specifies the target site in its spacer sequence, but also encodes the desired mutation in an extension that is typically at the 3′ end of the tracrRNA. Upon target binding, the Cas9 nuclease domain nicks the PAM-containing DNA strand, where upon the newly liberated 3′ end at the target DNA site is used to prime reverse transcription using the extension in the guide RNA as a template. Thereby the mutation is transcribed into the genomic DNA.

In some embodiments, the first oligonucleotide encoding a mutation of interest is part of the guide RNA capable of hybridizing to the genomic target region. In some embodiments, the second oligonucleotide encoding the synonymous mutation is part of the guide RNA capable of hybridizing to the genomic target region. Said first and second oligonucleotides are preferably provided as part of separate guide RNAs capable of hybridizing to the genomic target region.

In some embodiments, one or more of the first oligonucleotide is single-stranded. In some embodiments, one or more of the second oligonucleotide is single-stranded. In some embodiments, one or more of the first oligonucleotide is double-stranded. In some embodiments, one or more of the second oligonucleotide is double-stranded. In some embodiments, one or more of the first oligonucleotide is modified, such as by introduction of one or more phosphorothioate bonds to inhibit oligonucleotide degradation by nucleases. In some embodiments, one or more of the second oligonucleotide is modified, such as by introduction of one or more phosphorothioate bonds to inhibit oligonucleotide degradation by nucleases.

Nucleases Useful for the Current Methods

Any nuclease proficient at generating single- or double-stranded DNA breaks in a target nucleic acid sequence may be used for the described methods. Said nuclease may be directed to cleave the target nucleic acid sequence by a targeting means. The first or the second oligonucleotide can then be introduced at, or copied into, the site of the break.

In some embodiments the cells comprise two or more alleles comprising the target nucleic acid sequence. Without being bound by theory, cells wherein the first or the second oligonucleotide have been introduced at the site of the break of one allele will nearly always have indels introduced in the other allele(s), leading to a deleted, truncated or otherwise non-functional protein being expressed from said other alleles, or the mRNA from said allele may be removed by non-sense mediated mRNA degradation to eliminate protein expression. Such cells will thus effectively only express a likely functional protein from the allele wherein mutation was introduced by the first or second oligonucleotide. This enables assessment of the effects of the mutation without the possibility of complementation from a wild type allele (restoration of the wild type phenotype), such as wherein the wild type gene product is dominant.

In some embodiments, the nuclease comprises or consist of a CRISPR/Cas nuclease and the targeting means comprise or consist of a guide RNA capable of hybridizing to the genomic target region.

In some embodiments, the nuclease is a CRISPR/Cas nuclease, such as the Streptococcus pyogenes Cas9 nuclease of SEQ ID NO: 1, a functional variant thereof which retains nuclease activity, or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. In some embodiments, said nuclease is encoded by a polynucleotide comprising or consisting of SEQ ID NO: 6 or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the nuclease is codon-optimised. For example, the nuclease is a human, codon-optimized Streptococcus pyogenes Cas9 (D10A) nickase, such as the human, codon-optimized Streptococcus pyogenes Cas9 (D10A) nickase of SEQ ID NO: 2, a functional variant thereof which retains nickase activity, or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. In some embodiments, said nuclease is encoded by a polynucleotide comprising or consisting of SEQ ID NO: 7 or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the nuclease is a Francisella novicida Cas12a nuclease, such as the Francisella novicida Cas12a nuclease of SEQ ID NO: 3, a functional variant thereof which retains nuclease activity, or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. In some embodiments, said nuclease is encoded by a polynucleotide comprising or consisting of SEQ ID NO: 8 or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the nuclease is a Francisella novicida Cas12a nuclease, such as MAD7 or a functional variant thereof which retains nuclease activity.

In some embodiments, the nuclease is a Cas9-NG nuclease, such as the Cas9-NG nuclease of SEQ ID NO: 5, a functional variant thereof which retains nuclease activity, or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. In some embodiments, said nuclease is encoded by a polynucleotide comprising or consisting of SEQ ID NO: 4 or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

The methods as disclosed herein may readily be used with nuclease prime editors.

In some embodiments, the nuclease is a prime editor, such as the prime editor PE2 of SEQ ID NO: 9, a functional variant thereof which retains nickase and reverse transcriptase activity, or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. In some embodiments, said nuclease is encoded by a polynucleotide comprising or consisting of SEQ ID NO: 10 or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, any one of the nucleases as described herein may be codon-optimized for the cell. Thus, in some embodiments, the nuclease is codon-optimized for human cells. In some embodiments, the CRISPR/Cas nuclease, such as the Streptococcus pyogenes Cas9 nuclease of SEQ ID NO: 1 or a functional variant thereof, is codon-optimized for human cells. In some embodiments, the CRISPR/Cas nuclease, such as the Francisella novicida Cas12a nuclease of SEQ ID NO: 3 or a functional variant thereof, or such as MAD7 or a functional variant thereof, is codon-optimized for human cells. In some embodiments, the CRISPR/Cas nuclease, such as the Cas9-NG nuclease of SEQ ID NO: 5 or a functional variant thereof, is codon-optimized for human cells. In some embodiments, the CRISPR/Cas nuclease, such as the prime editor PE2 of SEQ ID NO: 9 or a functional variant thereof, is codon-optimized, for example is codon-optimized for human cells.

In some embodiments, the nuclease and the targeting means comprise or consist of a transcription activator-like effector nuclease (TALEN) consisting of a DNA cleavage domain and a DNA-binding domain. Methods of how to design TALENs in order to cut a given target are known to persons skilled in the art and are also described in Sanjana et al., 2012.

In some embodiments, the nuclease and the targeting means comprise or consist of a zinc finger nuclease consisting of a DNA cleavage domain and a DNA-binding domain. Methods of how to design zinc finger nucleases in order to cut a given target are known to persons skilled in the art and are also described in Carroll et al., 2006 and Wright et al., 2006.

In some embodiments, the nuclease and the targeting means comprise or consist of a meganuclease. Methods of how to design meganucleases in order to cut a given target are known to persons skilled in the art and are also described in Silva et al., 2011.

Selection of Successfully Transfected and/or Genetically Modified Cells

Although a step of selecting cells in the population that have been successfully transfected with the nuclease, or which have successfully integrated either the first or the second oligonucleotide in the target nucleic acid, is not required for the presently disclosed methods, it may nevertheless be desirable to include such a step. Such a step may serve to reduce the background or noise pertaining to amplification errors of the target sequence in non-transfected cells.

Thus, in some embodiments, step ii) of the method as described herein further comprises selecting the cells in which the nuclease or the polynucleotide encoding said nuclease has been introduced, thereby obtaining a subpopulation of cells enriched in cells in which the mutation of interest and/or the synonymous mutation has been introduced in the target nucleic acid sequence.

In yet other embodiments, step ii) of the method as described herein further comprises selecting the cells in which one of the first oligonucleotide or the second oligonucleotide has been introduced, thereby obtaining a mixed population of cells comprising cells in which the mutation of interest has been introduced in the target nucleic acid sequence, and cells in which the synonymous mutation has been introduced in the target nucleic acid sequence.

Said selection step may be performed by methods known in the art, such as by co-expression of a resistance gene together with the nuclease, e.g. from a transfected plasmid, and adding to the cell growth medium the compound that the cells become resistant to by expressing said gene, or such as by including a fluorescent tag on either the nuclease or either of the oligonucleotides, whereby the cells may be cell sorted for the fluorescent signal, e.g. by FACS.

Parameters for Assessment of the Effects of the Mutation of Interest in a Cell

The disclosed methods are useful for assessing the effects of mutations on a broad range of cell parameters. These parameters may be classified as being a temporal parameter, i.e. related to a change over time, and/or a spatial parameter, i.e. related to a specific location in physical space.

In some embodiments the parameter of interest is a cellular response to a compound or an external stimulus, such as temperature, drought or pressure, and the medium of step iii) of the present methods comprises said compound or step iii) additionally comprises subjecting the cell population to the external stimulus, and the cellular response is a temporal parameter or a spatial parameter. In some embodiments, the compound is a therapeutic agent or a candidate therapeutic agent, a virus or a viral agent, a pathogen, an active agent, a metabolite or a cell signaling molecule. In some embodiments, the external stimulus is a physical stimulus. In some embodiments the external stimulus is heat. In some embodiments the external stimulus is cold. In some embodiments the external stimulus is drought. In some embodiments the external stimulus is UV radiation.

Temporal Parameters

In some embodiments, the parameter of interest is a temporal parameter, and the method for assessing the effects of a mutation of interest in a cell comprises the steps of:
i) providing a cell population comprising a target nucleic acid sequence;
ii) introducing in at least some of the cells of the cell population:
   a) a nuclease or a polynucleotide encoding said nuclease, wherein the nuclease is capable of generating one or more single-strand breaks (SSBs) or double-strand breaks (DSBs) in a target nucleic acid sequence, and targeting means directing the nuclease to the target nucleic acid sequence, whereby the nuclease is capable of binding to a binding region of the target nucleic acid sequence, and whereby the nuclease is capable of generating one or more single-stranded breaks (SSBs) or double-strand breaks (DSBs) in the target nucleic acid sequence;
   b) a first oligonucleotide comprising a mutation of interest, preferably a non-silent mutation, preferably a non-synonymous mutation, wherein said non-synonymous mutation introduces a change in the encoded amino acid sequence compared to the amino acid sequence encoded by the target nucleic acid sequence, and otherwise identical to or complementary to said target nucleic acid sequence, wherein said mutation of interest preferably lies within the binding region of said nuclease; and
   c) a second oligonucleotide comprising a synonymous mutation, wherein said synonymous mutation preferably lies within the binding region of said nuclease, and wherein said synonymous mutation introduces no change in the encoded amino acid sequence compared to the amino acid sequence encoded by the target nucleic acid sequence;
   whereby said first oligonucleotide or said second oligonucleotide is integrated in, or copied into, the target nucleic acid sequence of at least some of the cells, thereby obtaining a mixed population of cells comprising cells in which the mutation of the first or the second oligonucleotides has not been introduced, cells in which only the mutation of the first oligonucleotide has been introduced, and cells in which only the mutation of the second oligonucleotide has been introduced;
iii) incubating the mixed population of cells in a medium for a determined duration, under conditions allowing a parameter of interest to be monitored, wherein the parameter of interest is a temporal parameter and/or a spatial parameter;
iv) determining the effect of the mutation of interest on the parameter of interest, wherein:
   A. determining the effect of the mutation of interest on the parameter of interest comprises the steps of:
      v) determining an initial ratio of cells in which the mutation of interest has been introduced in the target nucleic acid sequence to cells in which the synonymous mutation has been introduced in the target nucleic acid sequence, wherein the initial ratio of cells is determined at an initial time point; determining a subsequent ratio of cells in which the mutation of interest has been introduced in the target nucleic acid sequence to cells in which the synonymous mutation has been introduced in the target nucleic acid sequence, wherein the subsequent ratio of cells is determined at a subsequent time point; and determining a change in ratio between the initial ratio and the subsequent ratio; and
      vi) correlating said change in ratio to the parameter of interest, thereby assessing the effect of the mutation on the parameter of interest.

In some embodiments, the temporal parameter is cell proliferation. In some embodiments, the temporal parameter is cell growth. In some embodiments, the temporal parameter is anchorage-independent cell growth. In some embodiments, the temporal parameter is contribution to tumor growth. In some embodiments, the temporal parameter is fitness. In some embodiments, the temporal parameter is cell motility. In some embodiments, the temporal parameter is cell invasiveness. In some embodiments, the temporal parameter is cellular metabolism. In some embodiments, the temporal parameter is DNA damage. In some embodiments, the temporal parameter is expression levels of pre-defined genes and/or proteins. In some embodiments, the temporal parameter is resistance to a compound. In some embodiments, the temporal parameter is sensitivity to a compound. In some embodiments, the temporal parameter is production of a compound. In some embodiments, the temporal parameter is anoikis. In some embodiments, the temporal parameter is senescence. In some embodiments, the temporal parameter is contact inhibition. In some embodiments, the temporal parameter is apoptosis.

In some embodiments, the parameter of interest is a temporal parameter of interest, and if the initial ratio of cells in which the mutation of interest has been introduced in the target nucleic acid sequence to cells in which the synonymous mutation has been introduced in the target nucleic acid sequence, is lower than the subsequent ratio of cells in which the mutation of interest has been introduced in the target nucleic acid sequence to cells in which the synonymous mutation has been introduced in the target nucleic acid sequence, the mutation is characterised as a mutation having a positive effect on the temporal parameter.

In some embodiments, the parameter of interest is a temporal parameter of interest, and if the initial ratio of cells in which the mutation of interest has been introduced in the target nucleic acid sequence to cells in which the synonymous mutation has been introduced in the target nucleic acid sequence, is greater than the subsequent ratio of cells in which the mutation of interest has been introduced in the target nucleic acid sequence to cells in which the synonymous mutation has been introduced in the target nucleic acid sequence, the mutation is characterised as a mutation having a negative effect on the temporal parameter.

In some embodiments, the parameter of interest is a temporal parameter of interest, and if the initial ratio of cells in which the mutation of interest has been introduced in the target nucleic acid sequence to cells in which the synonymous mutation has been introduced in the target nucleic acid sequence, is substantially the same as the subsequent ratio of cells in which the mutation of interest has been introduced in the target nucleic acid sequence to cells in which the synonymous mutation has been introduced in the target nucleic acid sequence, the mutation is characterised as a mutation having no effect on the temporal parameter.

As mentioned herein above, the parameter of interest may be a cellular response to a compound or an external stimulus, such as temperature, drought or pressure, and the medium of step iii) of the disclosed methods comprises said compound or step iii) additionally comprises subjecting the cell population to the external stimulus. The effect of the mutation of interest on the parameter can then be determined by exposing the cells to conditions for assessing the parameter, for example the compound or the external stimulus.

In some embodiments, the cellular response is monitored as a temporal parameter, and if the initial ratio of cells is lower than the subsequent ratio of cells, it indicates a positive effect of the mutation of interest on the cellular response to the compound. In some embodiments, the cellular response is monitored as a temporal parameter, and if the initial ratio of cells is greater than the subsequent ratio of cells, it indicates a negative effect of the mutation of interest on the cellular response to the compound. In some embodiments, the cellular response is monitored as a temporal parameter, and if the initial and the subsequent ratio of the cells are substantially the same, it indicates no effect of the mutation of interest on the cellular response to the compound.

Spatial Parameters

In some embodiments, the parameter of interest is a spatial parameter, and the method for assessing the effects of a mutation of interest in a cell comprises the steps of:
i) providing a cell population comprising a target nucleic acid sequence;
ii) introducing in at least some of the cells of the cell population:
   a) a nuclease or a polynucleotide encoding said nuclease, wherein the nuclease is capable of generating one or more single-strand breaks (SSBs) or double-strand breaks (DSBs) in a target nucleic acid sequence, and targeting means directing the nuclease to the target nucleic acid sequence, whereby the nuclease is capable of binding to a binding region of the target nucleic acid sequence, and whereby the nuclease is capable of generating one or more single-stranded breaks (SSBs) or double-strand breaks (DSBs) in the target nucleic acid sequence;
   b) a first oligonucleotide comprising a mutation of interest, preferably a non-silent mutation, preferably a non-synonymous mutation, wherein said non-synonymous mutation introduces a change in the encoded amino acid sequence compared to the amino acid sequence encoded by the target nucleic acid sequence, and otherwise identical to or complementary to said target nucleic acid sequence, wherein said mutation of interest preferably lies within the binding region of said nuclease; and
   c) a second oligonucleotide comprising a synonymous mutation, wherein said synonymous mutation preferably lies within the binding region of said nuclease, and wherein said synonymous mutation introduces no change in the encoded amino acid sequence compared to the amino acid sequence encoded by the target nucleic acid sequence;
   whereby said first oligonucleotide or said second oligonucleotide is integrated in, or copied into, the target nucleic acid sequence of at least some of the cells, thereby obtaining a mixed population of cells comprising cells in which the mutation of the first or the second oligonucleotides has not been introduced, cells in which only the mutation of the first oligonucleotide has been introduced, and cells in which only the mutation of the second oligonucleotide has been introduced;
iii) incubating the mixed population of cells in a medium for a determined duration, under conditions allowing a parameter of interest to be monitored, wherein the parameter of interest is a temporal parameter and/or a spatial parameter;
iv) determining the effect of the mutation of interest on the parameter of interest, wherein:
   B. determining the effect of the mutation of interest on the parameter of interest comprises the steps of:
      v) defining and/or spatially separating subpopulations of cells on the basis of said spatial parameter of interest in each subpopulation, preferably wherein the spatial parameter of interest is different in each subpopulation;
      vi) determining, for each subpopulation, a ratio of cells in which the mutation of interest has been introduced in the target nucleic acid sequence to cells in which the synonymous mutation has been introduced in the target nucleic acid sequence; and
      vii) correlating said ratio to the measured spatial parameter of interest for each subpopulation;
         thereby assessing the effect of the mutation on the parameter of interest.

In some embodiments, the spatial parameter is cell proliferation. In some embodiments, the spatial parameter is cell growth. In some embodiments, the spatial parameter is fitness. In some embodiments, the spatial parameter is cell motility. In some embodiments, the spatial parameter is cell invasiveness. In some embodiments, the spatial parameter is cellular metabolism. In some embodiments, the spatial parameter is cell differentiation. In some embodiments, the spatial parameter is DNA damage. In some embodiments, the spatial parameter is expression levels of pre-defined genes and/or proteins. In some embodiments, the spatial parameter is resistance to a compound. In some embodiments, the spatial parameter is sensitivity to a compound. In some embodiments, the spatial parameter is production of a compound. In some embodiments, the spatial parameter is anoikis. In some embodiments, the spatial parameter is senescence. In some embodiments, the spatial parameter is apoptosis. In some embodiments, the spatial parameter is DNA methylation. In some embodiments, the spatial parameter is protein post-translational modification.

Spatial parameters may be measured at certain spatial locations of interest, e.g. a subpopulation present on only one side of a cell-permeable membrane, a subpopulation of physically isolated cells that have been FACS-sorted for expression of a cell marker at a higher level compared to a reference cell population, or a specific organ in a test animal having received a xenograft of a transfected cell population, and the ratio of cells in which the synonymous mutation has been introduced in the target nucleic acid sequence to cells in which the mutation of interest has been introduced in the target nucleic acid sequence is then measured in the subpopulation present at said spatial location of interest and compared to the same measurement in a subpopulation present at a reference location. The subpopulation of step B.v) may thus also be defined or separated from other subpopulations on the basis of the value of a certain spatial parameter, e.g. presence on one side of a cell-permeable membrane, expression of a cell marker at a higher level compared to a reference cell population that can be measured by e.g. FACS, or presence in a specific organ in an animal having received a xenograft of a transfected cell population.

Thus, in some embodiments, step B.v) of the disclosed methods comprises defining the subpopulations on the basis of a cell property, such as the ability of the cells to migrate or invade, optionally wherein the cell property is the spatial parameter of interest.

In some embodiments, each subpopulation of step B.v) and/or each reference population of the disclosed methods comprises one or more cells, such as a single cell or a plurality of cells.

In some embodiments, step B.v) of the methods as disclosed herein comprises defining at least one subpopulation of cells and a reference subpopulation of cells from the mixed population on the basis of the spatial parameter of interest, and if the ratio of cells in which the mutation of interest has been introduced in the target nucleic acid sequence to cells in which the synonymous mutation has been introduced in the target nucleic acid sequence in said subpopulation is greater than the ratio of cells in which the mutation of interest has been introduced in the target nucleic acid sequence to cells in which the synonymous mutation has been introduced in the target nucleic acid sequence in a reference subpopulation, the mutation is characterised as a mutation having a positive effect on the spatial parameter.

In some embodiments, step B.v) of the methods as disclosed herein comprises defining at least one subpopulation of cells and a reference subpopulation of cells from the mixed population on the basis of the spatial parameter of interest, and if the ratio of cells in which the mutation of interest has been introduced in the target nucleic acid sequence to cells in which the synonymous mutation has been introduced in the target nucleic acid sequence in said subpopulation is lower than the ratio of cells in which the mutation of interest has been introduced in the target nucleic acid sequence to cells in which the synonymous mutation has been introduced in the target nucleic acid sequence in a reference subpopulation, the mutation is characterised as a mutation having a negative effect on the spatial parameter.

In some embodiments, step B.v) of the methods as disclosed herein comprises defining at least one subpopulation of cells and a reference subpopulation of cells from the mixed population on the basis of the spatial parameter of interest, and if the ratio of cells in which the mutation of interest has been introduced in the target nucleic acid sequence to cells in which the synonymous mutation has been introduced in the target nucleic acid sequence in said subpopulation is substantially the same as the ratio of cells in which the mutation of interest has been introduced in the target nucleic acid sequence to cells in which the synonymous mutation has been introduced in the target nucleic acid sequence in a reference subpopulation, the mutation is characterised as a mutation having no effect on the spatial parameter.

In some embodiments, step B.v) further comprises spatially separating said at least one subpopulation from the reference subpopulation.

In some embodiments, step B.v) of the disclosed methods comprises defining at least one subpopulation of cells and a reference subpopulation of cells from the mixed population on the basis of the spatial parameter of interest and spatially separating said at least one subpopulation from the reference subpopulation, and if the ratio of cells in said subpopulation is greater than in said reference subpopulation, the mutation is characterised as a mutation having a positive effect on the spatial parameter, the mutation is characterised as a mutation having a positive effect on the spatial parameter.

In some embodiments, step B.v) of the methods as disclosed herein comprises defining at least one subpopulation of cells and a reference subpopulation of cells from the mixed population on the basis of the spatial parameter of interest and spatially separating said at least one subpopulation from the reference subpopulation, and if the ratio of cells in said subpopulation is lower than the ratio of said reference subpopulation, the mutation is characterised as a mutation having a negative effect on the spatial parameter, the mutation is characterised as a mutation having a negative effect on the spatial parameter.

In some embodiments, step B.v) of the disclosed methods comprises defining at least one subpopulation of cells and a reference subpopulation of cells from the mixed population on the basis of the spatial parameter of interest and spatially separating said at least one subpopulation from the reference subpopulation, and if the ratio of cells in said subpopulation is substantially the same as the ratio of cells in said reference subpopulation, the mutation is characterised as a mutation having no effect on the spatial parameter.

As mentioned herein above, the parameter of interest may be a cellular response to a compound or an external stimulus, and the medium of step iii) of the disclosed methods comprises said compound.

In some embodiments, the cellular response is monitored as a spatial parameter, wherein for each subpopulation, a ratio of said subpopulation greater than the ratio of a reference subpopulation indicates that the mutation has a positive effect on the cellular response to the compound.

In some embodiments, the cellular response is monitored as a spatial parameter, wherein for each subpopulation a ratio of said subpopulation less than the ratio of a reference subpopulation indicates that the mutation has a negative effect on the cellular response to the compound.

In some embodiments, the cellular response is monitored as a spatial parameter, wherein for each subpopulation a ratio of said subpopulation substantially the same as the ratio of a reference subpopulation indicates that the mutation has no effect on the cellular response to the compound.

To define the value of the parameter of interest, it may be desirable to compare with a reference population, such as a population that has not been transfected with any of the oligonucleotides as described herein above in the section ‘Oligonucleotides useful for the current methods’. Said reference population may also be cells on one side of a cell-permeable membrane, for example cells may be plated on one side of a cell-permeable membrane, and some cells of the cell population may display high invasiveness and/or motility and translocate through the membrane to the other side—in this case the cell population on the side in which the cells were originally plated may be used as the reference population. The reference population may also be an organ in an animal having received a xenograft of transfected cells, e.g. if the parameter of interest relates to which cells have migrated from the xenograft to the liver, cells from another organ, such as the spleen or lungs, may be used as the reference population.

Thus, in some embodiments, the spatial parameter of interest has a value of interest defined by comparison with a reference value of the same spatial parameter in a reference population. The values obtained for a reference population (e.g. side scatter as measured by FACS) may define a value of interest (e.g. what values for side scatter as measured by FACS would be of interest when compared to the reference population). The reference value of the same spatial parameter in a reference population may thus define a cutoff value for when the spatial parameter of interest has a value of interest.

In some embodiments, step B.v) of the disclosed methods comprises a step of spatially separating the cells to separate the mixed cell population into subpopulations on the basis of a cell marker, such as the presence or absence or a graded level of a cell marker, for example a cell marker for cell proliferation, cell growth, fitness, cell motility, cell invasiveness, cellular metabolism, cell differentiation, DNA damage, expression levels of pre-defined genes or proteins, resistance to a compound, sensitivity to a compound, production of a compound, anoikis, senescence, apoptosis, DNA methylation or protein post-translational modification, optionally wherein the cell marker is the spatial parameter of interest.

In some embodiments, the step of spatially separating the cells is performed by FACS. In some embodiments, step B.v) of the disclosed methods further comprises a step of staining the cells with an antibody marker, such as a fluorescently labelled antibody, prior to FACS-sorting the cells, whereby cells may be spatially separated based on the signal intensity of the antibody marker.

Systems for Assessing the Effects of a Mutation of Interest in a Cell

In one aspect, the present invention provides a system comprising:
i. a nuclease or a polynucleotide encoding said nuclease, wherein the nuclease is capable of generating one or more single-strand breaks (SSBs) or double-strand breaks (DSBs) in a target nucleic acid sequence, and targeting means directing the nuclease to the target nucleic acid sequence, whereby the nuclease is capable of binding to a binding region of the target nucleic acid sequence, and whereby the nuclease is capable of generating one or more single-stranded breaks (SSBs) or double-strand breaks (DSBs) in the target nucleic acid sequence;
ii. a first oligonucleotide comprising a mutation of interest, preferably a non-silent mutation, preferably a non-synonymous mutation, wherein said non-synonymous mutation introduces a change in the encoded amino acid sequence compared to the amino acid sequence encoded by the target nucleic acid sequence, and otherwise identical to or complementary to said target nucleic acid sequence, wherein said mutation of interest preferably lies within the binding region of said nuclease; and
iii. a second oligonucleotide comprising a synonymous mutation, wherein said synonymous mutation preferably lies within the binding region of said nuclease, and wherein said synonymous mutation introduces no change in the encoded amino acid sequence compared to the amino acid sequence encoded by the target nucleic acid sequence.

Such systems are particularly well suited for performing the methods as described herein.

In some embodiments, the synonymous mutation is a single base change compared to the target nucleic acid sequence. In some embodiments, the mutation of interest is a single base change compared to the target nucleic acid sequence.

In some embodiments, the system further comprises primers for amplifying a region comprising the target nucleic acid sequence, such as a forward and a reverse primer allowing amplification by PCR.

In some embodiments, one or more of the first oligonucleotide, the second oligonucleotide, the targeting means and the polynucleotide encoding the nuclease are comprised within a vector, such as a plasmid.

Oligonucleotides Useful for the Current Systems

Useful oligonucleotides for the disclosed systems include oligonucleotides described herein above in the section ‘Oligonucleotides useful for the current methods’.

The systems as described herein may be used for assessing the effects of several different mutations of interest in a cell at the same time. Thus, the present systems may be used for multiplex assays, wherein the effects of multiple mutations are assessed simultaneously in a single experiment.

Thus, in some embodiments, the first oligonucleotide is a plurality of first oligonucleotides each comprising a pre-determined mutation of interest. In some embodiments, the second oligonucleotide is a plurality of second oligonucleotides each comprising a pre-determined synonymous mutation.

In some embodiments, said second oligonucleotides are otherwise identical to said first oligonucleotides. In some embodiments, said second oligonucleotides are the same lengths. In some embodiments, said first oligonucleotides are the same lengths. In some embodiments, said second oligonucleotides are of different lengths. In some embodiments, said first oligonucleotides are of different lengths. In some embodiments, said second oligonucleotides are the same length as said first oligonucleotides. In some embodiments, said second oligonucleotides are of different length to said first oligonucleotides.

In some embodiments, the first oligonucleotide comprises a plurality of pre-determined mutations of interest and wherein the second oligonucleotide comprises a plurality of pre-determined synonymous mutations.

In some embodiments, the plurality of mutations is at least 2 different mutations, such as at least 3 different mutations, such as at least 4 different mutations, such as at least different mutations, such as at least 6 different mutations, such as at least 7 different mutations, such as at least 8 different mutations, such as at least 9 different mutations, such as at least 10 different mutations, such as at least 15 different mutations, such as at least 20 different mutations, such as at least 25 different mutations, such as at least different mutations, such as at least 35 different mutations, such as at least 40 different mutations, such as at least 45 different mutations, such as at least 50 different mutations or more.

In some embodiments, the target nucleic acid sequence is within a gene, a promoter or an enhancer of a gene, wherein the gene is or is suspected to be an oncogene. In some embodiments, said gene is or is suspected to be a proto-oncogene. In some embodiments, said gene is or is suspected to be a tumor suppressor gene. In some embodiments, said gene is or is suspected to be a gene encoding an enzyme such as an enzyme involved in the production of a compound such as a metabolite, a resistance gene, such as a gene involved in resistance to a compound, a pharmaceutical compound, or a pathogen such as a virus. In some embodiments, said gene is or is suspected to be a gene encoding a protein involved in cellular fitness and/or growth. In some embodiments, said gene is or is suspected to be a gene encoding a protein for any cell function, a microRNA or a long non-coding RNA. The target nucleic acid may be genomic, i.e. comprised in the genome of the cells comprised within the cell population, or it may be extrachromosomal, e.g. on a vector or plasmid or may be comprised in DNA of an infected pathogen or invading organism within another cell.

Nucleases Useful for the Current Systems

Useful oligonucleotides for the disclosed systems include oligonucleotides described herein above in the section ‘Oligonucleotides useful for the current methods’.

In some embodiments, the nuclease comprises or consist of a CRISPR/Cas nuclease and the targeting means comprise or consist of a guide RNA capable of hybridizing to the target nucleic acid sequence.

In some embodiments, the nuclease is a CRISPR/Cas nuclease, such as the Streptococcus pyogenes Cas9 nuclease of SEQ ID NO: 1, a functional variant thereof which retains nuclease activity, or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. In some embodiments, said nuclease is encoded by a polynucleotide comprising or consisting of SEQ ID NO: 6 or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the nuclease is codon-optimised. For example, the nuclease is a human, codon-optimized Streptococcus pyogenes Cas9 (D10A) nickase, such as the human, codon-optimized Streptococcus pyogenes Cas9 (D10A) nickase of SEQ ID NO: 2, a functional variant thereof which retains nickase activity, or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. In some embodiments, said nuclease is encoded by a polynucleotide comprising or consisting of SEQ ID NO: 7 or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the nuclease is a Francisella novicida Cas12a nuclease, such as the Francisella novicida Cas12a nuclease of SEQ ID NO: 3, a functional variant thereof which retains nuclease activity, or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. In some embodiments, said nuclease is encoded by a polynucleotide comprising or consisting of SEQ ID NO: 8 or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, the nuclease is a Francisella novicida Cas12a nuclease, such as MAD7 or a functional variant thereof, which retains nuclease activity.

In some embodiments, the nuclease is a Cas9-NG nuclease, such as the Cas9-NG nuclease of SEQ ID NO: 5, a functional variant thereof which retains nuclease activity, or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. In some embodiments, said nuclease is encoded by a polynucleotide comprising or consisting of SEQ ID NO: 4 or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

The systems as disclosed herein may readily be used with nuclease prime editors.

In some embodiments, the nuclease is a prime editor, such as the prime editor PE2 of SEQ ID NO: 9, a functional variant thereof which retains nickase and reverse transcriptase activity, or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto. In some embodiments, said nuclease is encoded by a polynucleotide comprising or consisting of SEQ ID NO: 10 or a homologue thereof having at least 80%, such as at least 81%, such as at least 82%, such as at least 83%, such as at least 84% such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology thereto.

In some embodiments, any one of the nucleases as described herein may be codon-optimized for the cell. Thus, in some embodiments, the nuclease is codon-optimized for human cells. In some embodiments, the CRISPR/Cas nuclease, such as the Streptococcus pyogenes Cas9 nuclease of SEQ ID NO: 1 or a functional variant thereof, is codon-optimized for human cells. In some embodiments, the CRISPR/Cas nuclease, such as the Francisella novicida Cas12a nuclease of SEQ ID NO: 3 or a functional variant thereof, or such as MAD7 or a functional variant thereof, is codon-optimized for human cells. In some embodiments, the CRISPR/Cas nuclease, such as the Cas9-NG nuclease of SEQ ID NO: 5 or a functional variant thereof, is codon-optimized for human cells. In some embodiments, the CRISPR/Cas nuclease, such as the prime editor PE2 of SEQ ID NO: 9 or a functional variant thereof, is codon-optimized for human cells.

In some embodiments, the nuclease and the targeting means comprise or consist of a transcription activator-like effector nuclease (TALEN) consisting of a DNA cleavage domain and a DNA-binding domain. In some embodiments, the nuclease and the targeting means comprise or consist of a zinc finger nuclease consisting of a DNA cleavage domain and a DNA-binding domain. In some embodiments, the nuclease and the targeting means comprise or consist of a meganuclease.

Host Cells or Host Cell Populations Comprising the System for Assessing the Effects of a Mutation of Interest in a Cell

In one aspect, the present invention provides a host cell comprising the system as described herein above in the section ‘Systems for assessing the effects of a mutation of interest in a cell’.

In some embodiments, the present invention provides a host cell comprising part of the system as described herein above in the section ‘Systems for assessing the effects of a mutation of interest in a cell’.
In another aspect, the present invention provides a host cell population comprising the system as described herein above in the section ‘Systems for assessing the effects of a mutation of interest in a cell’.

In some embodiments, the host cell is a bacterial cell. In some embodiments, the host cell is a eukaryotic cell such as a vertebrate cell, an invertebrate cell, a plant cell, a yeast cell or a fungal cell.

Uses of the Systems as Disclosed Herein for Assessing the Effects of a Mutation of Interest in a Cell

In one aspect, the present invention also provides uses of the systems as described herein above in the section ‘Systems for assessing the effects of a mutation of interest in a cell’ in a method for assessing the effects of a mutation of interest in a cell, wherein the method is as described herein above in the section ‘Methods for assessing the effects of a mutation of interest in a cell’.

In some embodiments, the cell is a mammalian cell such as a human cell. In some embodiments, the cell is a vertebrate cell. In some embodiments, the cell is an invertebrate cell. In some embodiments, the cell is a plant cell. In some embodiments, the cell is a yeast cell. In some embodiments, the cell is a fungal cell.

In some embodiments, the method is performed in vitro. In some embodiments, the method is performed in vivo.

### EXAMPLES

### Example 1—Example Uses of the Method

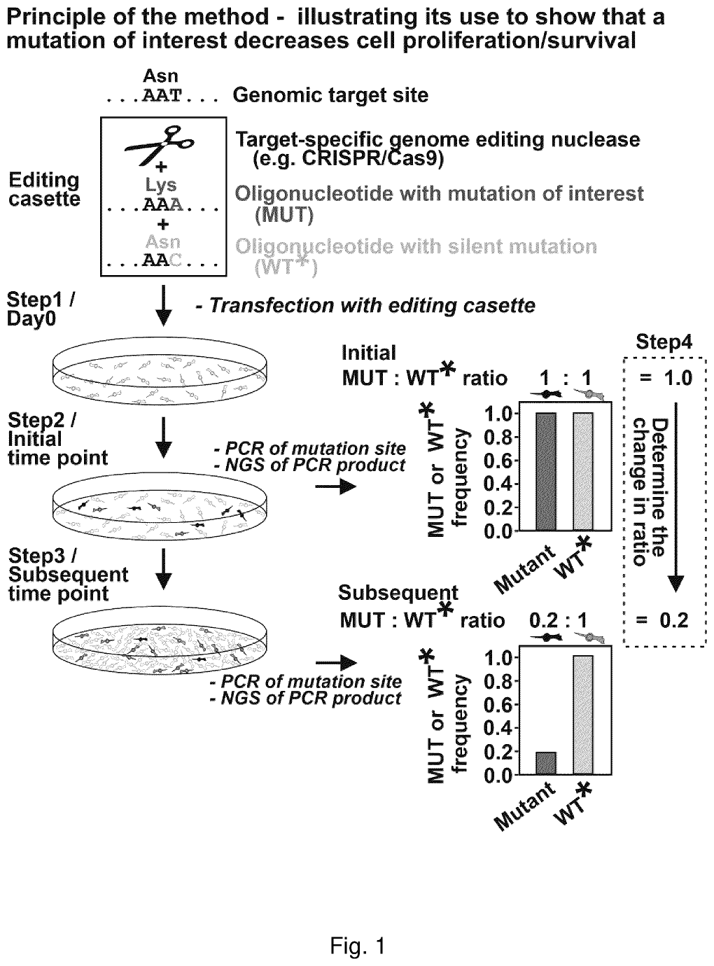

The principle of the method is illustrated in FIG. 1, exemplified with the use of the method to demonstrate that a mutation of interest decreases cell proliferation and/or survival, analysed as a temporal cell parameter. In the first step, a nuclease targeting a desired genomic target site containing an Asn residue to be mutated, an oligonucleotide with mutation of interest (MUT; intending to mutate the Asn to Lys residue) and an oligonucleotide with a synonymous mutation, serving as internal wild-type normalization control (WT*; replacing the parental Asn codon with another Asn codon) are co-introduced in a cell population. At an initial time point, an initial ratio of cells with knockin of mutation of interest to cells with knockin of the synonymous WT* mutation is determined on part of the cell population, preferably by genomic PCR amplification of the target site using primers flanking the region covered by the oligonucleotides and subsequent NGS of the PCR products. At a subsequent time point, a subsequent ratio is determined by same approach. If the ratio of MUT to WT* decreases between the initial and the subsequent time point, as in this example, then the mutation of interest has a negative effect on cell proliferation and/or survival.

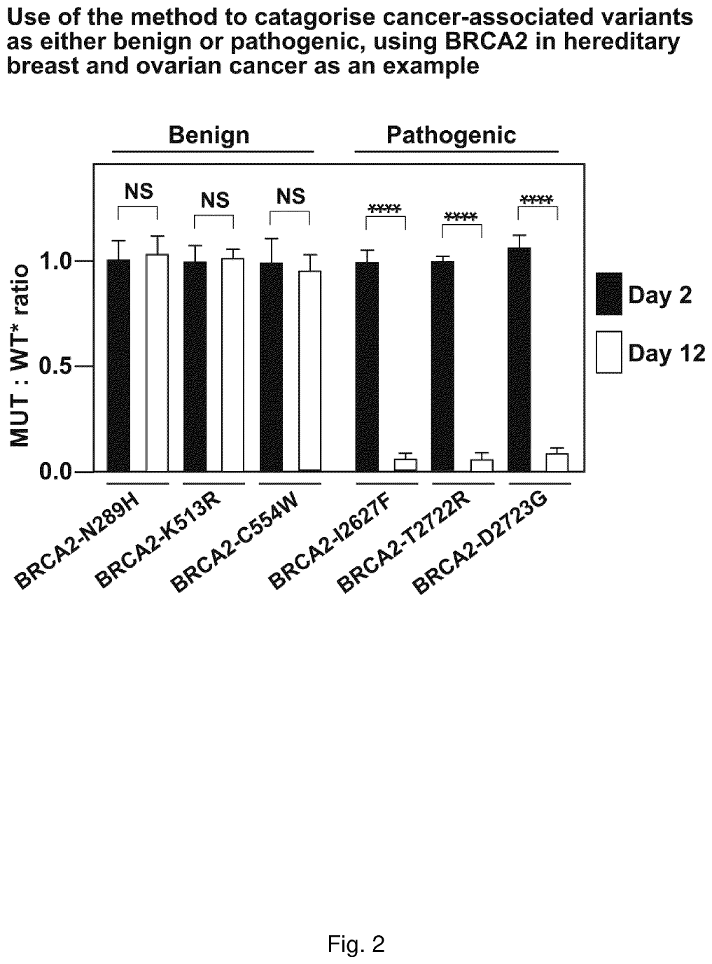

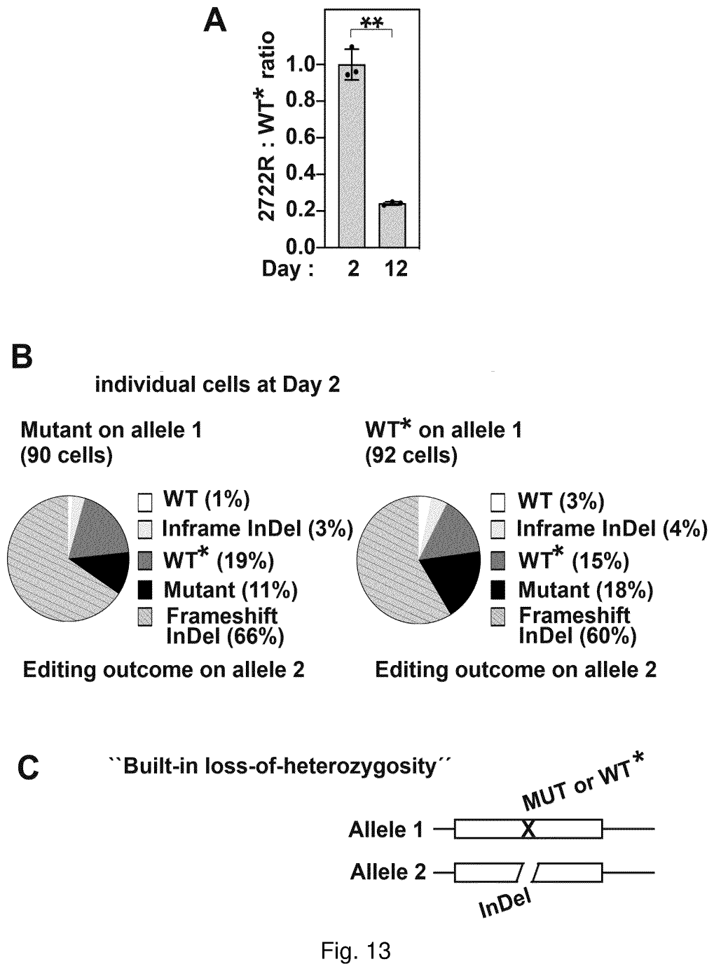

FIG. 2 shows an example of use of the method for functional genetic diagnosis of cancer. BRCA2 is one of >30 genes underlying hereditary breast and ovarian cancer. Disease-causing mutations result in loss-of-function of BRCA2 and decreased proliferation and survival in cells with such mutations. The ClinVar public database reports >5000 BRCA2 variants-of-unknown-significance (VUS) per November 2020, most of which were identified, when doctors have sequenced the DNA of patients with breast or ovarian cancer. A VUS is a variant, for which it is not known if it is benign (neutral) or pathogenic (disease-causing). To demonstrate that the method can be used for functional diagnosis of cancer-associated variants, we applied the method to 3 expert panel-assessed (i.e. known) benign and 3 expert panel-assessed (i.e. known) pathogenic BRCA2 variants, serving as test controls for proper functioning of our method. Specifically, we introduced the mutations in a population of the human breast epithelial MCF10A-BRCA2+/− cell line, using assay design, as described in the figure legend. “BRCA2+/−” indicates that in this cell line, the BRCA2 gene had been deleted on one allele using CRISPR/Cas9 in order to mimick the situation in patient breast cancer tumors, where loss-of-function variants generally manifest after loss of one normal allele (loss-of-heterozygosity). While the method indeed contains “built-in loss-of-heterozygosity” that is sufficient for research purpose analysis of loss-of-function variants in diploid cells (FIG. 13), the use of our method for functional clinical genetic diagnostics use, exemplified here, would typically involve the generation and use of cells with a defined genetic setting, such as BRCA2+/− cells for BRCA2 variant analysis. The results showed that the ratio of benign mutations to the corresponding synonymous control variants (WT*) did not decrease over time, as expected, because the benign mutations are neutral. By contrast, the ratio of pathogenic mutations to synonymous control variants (WT*) decreased over time, as expected, because the pathogenic mutations are loss-of-function, causing decreased cell proliferation and survival in the targeted cell population. In conclusion, the method can correctly catagorise BRCA2 variants as either benign or pathogenic within two weeks, and is thereby useful for functional genetic diagnosis of cancer.

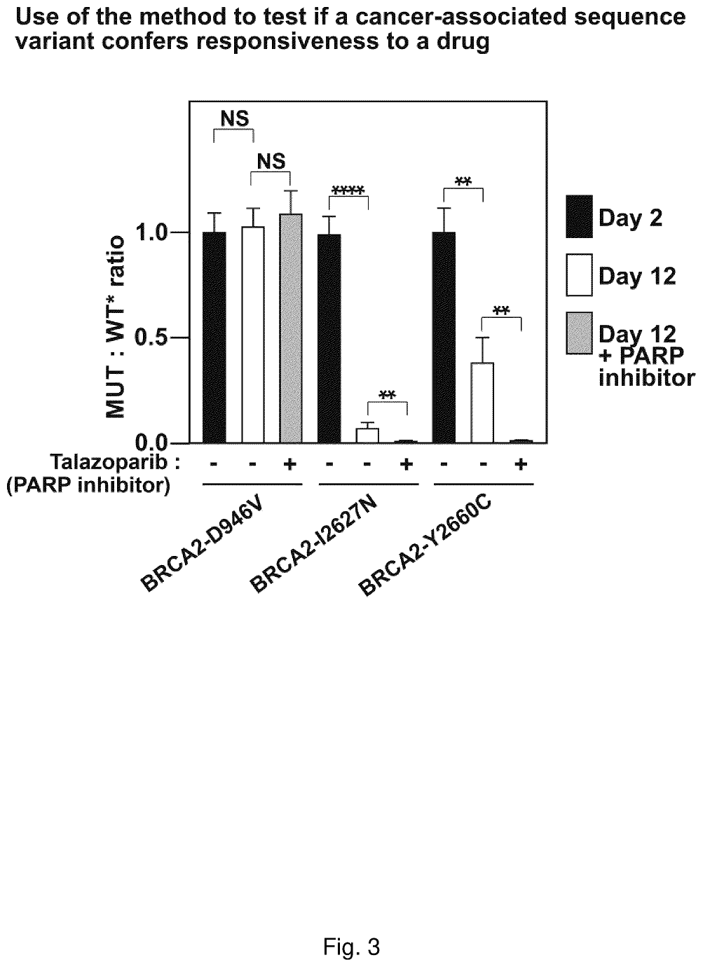

FIG. 3 demonstrates that the method can be used to test if a given cancer-associated variant confers responsiveness to a given drug, using PARP inhibitors as an example. BRCA2 loss-of-function mutations sensitize cells to inhibitors of PARP, which are used in clinical trials/in the clinic as a synthetic lethal personalized therapy for cancer patients with BRCA inactivating mutations. We analysed one neutral BRCA2 mutation (D946V) and two loss-of-function BRCA2 mutations (I2627N, Y2660C) in breast epithelial MCF10A-BRCA2+/− cells cultured in the absence or presence of PARP inhibitor. As expected, the PARP inhibitor had no effect on cells with the neutral BRCA2 mutation, whereas the PARP inhibitor greatly decreased cell proliferation and/or survival of cells with the loss-of-function BRCA2 mutations. In conclusion, the method can thus be used to determine if cancer patients with a given mutation will respond to a given drug. Furthermore, the method will thereby also be useful for the purpose of drug development, drug testing and stratification of cancer patients in clinical trials with new drugs.

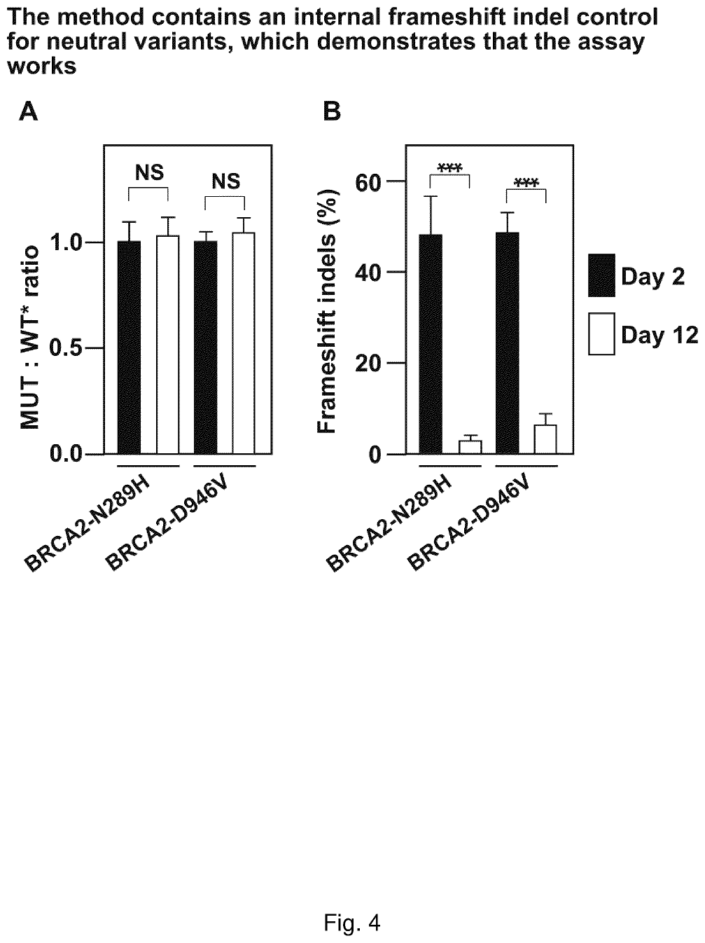

FIG. 4 illustrates that the method contains an internal control, which can show that apparently neutral variants are truly neutral. Thus, in addition to cells with knockin of the mutation of interest or the WT* synonymous mutation, a substantial fraction of cells will have introduction of a frameshifting indel (insertion/deletion) mutation, which are loss-of-function mutations. The fate of these cells over time can also be analysed in the same cell culture, where the neutral variant was investigated. As shown in FIG. 4, in the same MCF10A-BRCA2+/− cell culture, where the BRCA2 mutations N289H or D946V did not reduce proliferation and/or survival between the initial (Day 2) and subsequent (Day12), i.e. they behaved as neutral variants (FIG. 4A), the cells with frameshifting indels were strongly selected against, as evidenced from the depletion of cells with such indels over time (FIG. 4B). In conclusion, the internal indel control demonstrates that the assay worked during this experiment, meaning that the lack of effect of the N289H and D946V mutations is because they are truly neutral.

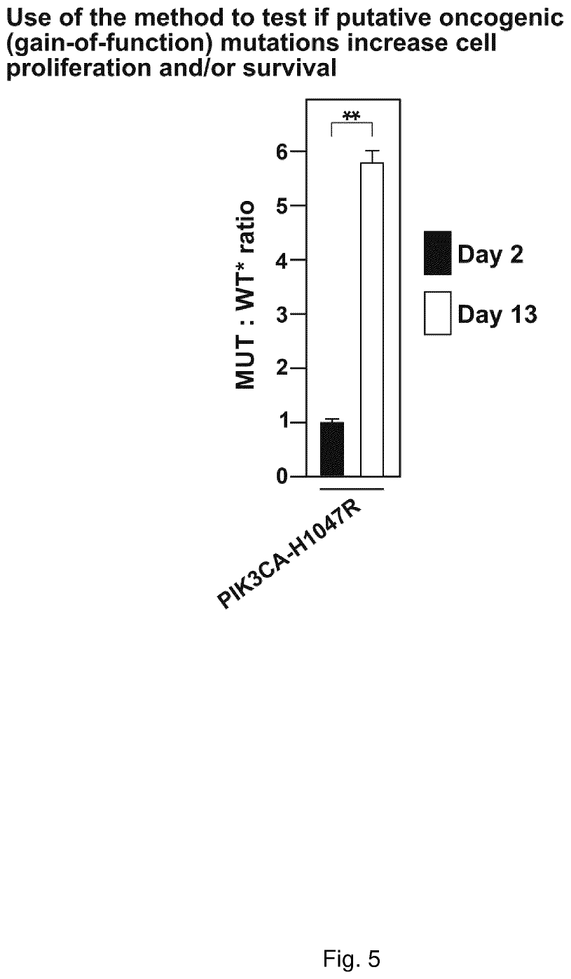

FIG. 5 demonstrates that the method can be used to test if putative oncogenic mutations indeed are gain-of-function mutations, for example by promoting cell proliferation and/or survival. In this example, a known oncogenic H1047R mutation in the oncogene PIK3CA was analysed in human MCF10A cells. The results showed that cells with this mutation were strongly selected for over time, when the cells, where cultured under serum- and growth factor-deprived conditions. In conclusion, the method can also be used to demonstrate a driver function of putative oncogenic mutations.

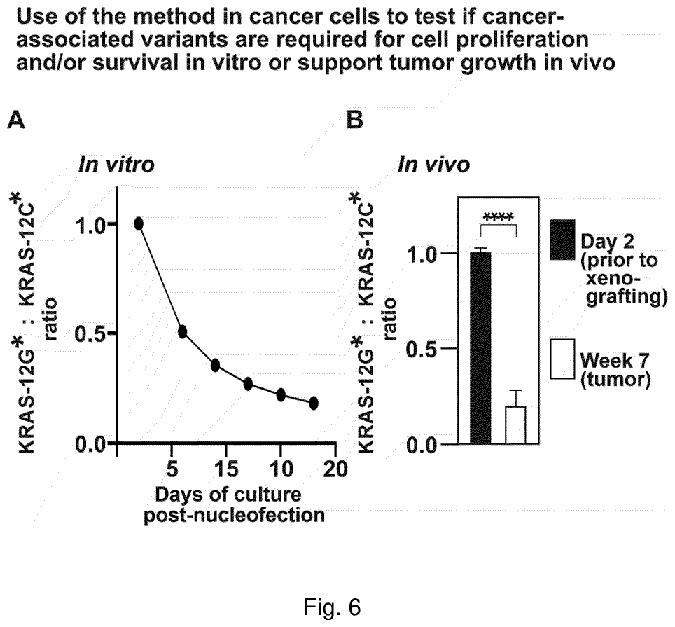

FIG. 6 illustrates the use of the method in human cancer cells to demonstrate that a given mutated gene is oncogenic driver in the cancer cells and that the gene is thereby a candidate drug target. The figure also illustrates that the method works in vitro as well as in vivo. Specifically, human lung cancer H358 cells harboring the oncogenic KRAS G12C driver mutations was subjected to analysis, in which 12C was mutated (corrected) back to wild-type 12G (mutation of interest; 12G*) or to an alternative synonymous 12C codon mutation (120*). The results showed that cells with corrected KRAS 12G* were selected against over time relative to cells with the synonymous control 12C*, both when cultured in vitro (FIG. 5A) and when cells were xenografted into nude mice (FIG. 5B). This demonstrates the dependency of the cancer cells on KRAS-12C, both for cell proliferation and/or survival and for tumor formation. In conclusion, the results demonstrate that the method can be used to identify oncogenic drivers and candidate drug targets for targeted cancer therapies, both in vitro and in cancer mouse models.

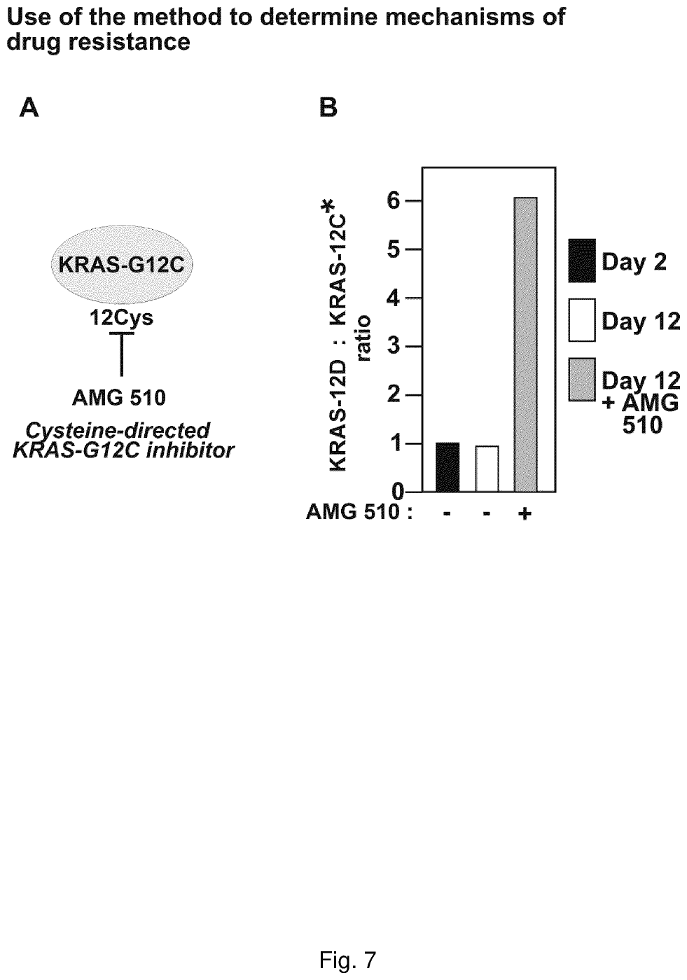

FIG. 7 shows the use of the method to determine mechanism of resistance to targeted anti-cancer drugs. Specifically, the recently developed compound AMG 510 targets the 12C residue in the KRAS-G12C oncogene and AMG 510 is increasingly being used to treat cancer patients harboring this mutation in their tumors. However, in a fraction of such patients undergoing this treatment, selection may arise for cancer cells, in which 12C has been mutated to another oncogenic KRAS mutation such as 12D. To test this hypothesis, we used the method to mutate KRAS-12C to 12D or the synonymous 12C* in H358 lung cancer cells and cultured the cells in the absence or presence of AMG 510. The method thereby revealed that cells harboring the KRAS-12D mutation were strongly selected for, when treated with AMG 510, demonstrating that the 12D mutation confers resistance to AMG 510. In conclusion, the method can be used to determine resistance mechanisms to targeted anti-cancer drugs, which is important for clinical management and drug development.

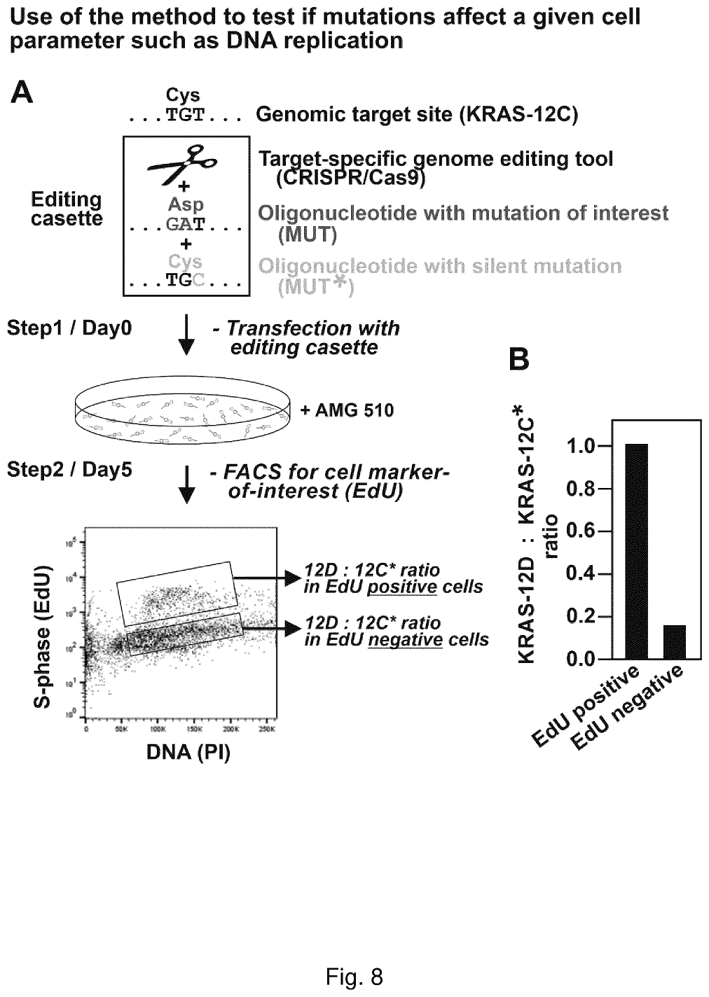

FIG. 8 demonstrates the use of the method to test, if a mutation of interest affects a specific (spatial) cell parameter, illustrated with the effect of the oncogenic KRAS-12D mutation to promote cell proliferation (DNA replication) in the presence AMG 510. In this version of the method, human lung cancer H358 cells subjected to mutation of KRAS-12C to 12D or the synonymous 12C* and cultured in the presence of AMG 510 for 5 days. After a 2 hours pulse with the S-phase (DNA replication) marker EdU, the cell culture was analysed by FACS for EdU. Subsequent analysis of the 12D to synonymous 12C* mutation ratios in cell subpopulations FACS isolated for either positive or negative EdU signal showed large enrichment of the 12D mutation in the former population, which thereby demonstrates that the KRAS-12D mutation suppresses the ability of AMG 510 to suppress cell proliferation. In conclusion, this experiment demonstrates that the method can link a specific genetic variant to a specific cell parameter, which can be monitored by FACS.

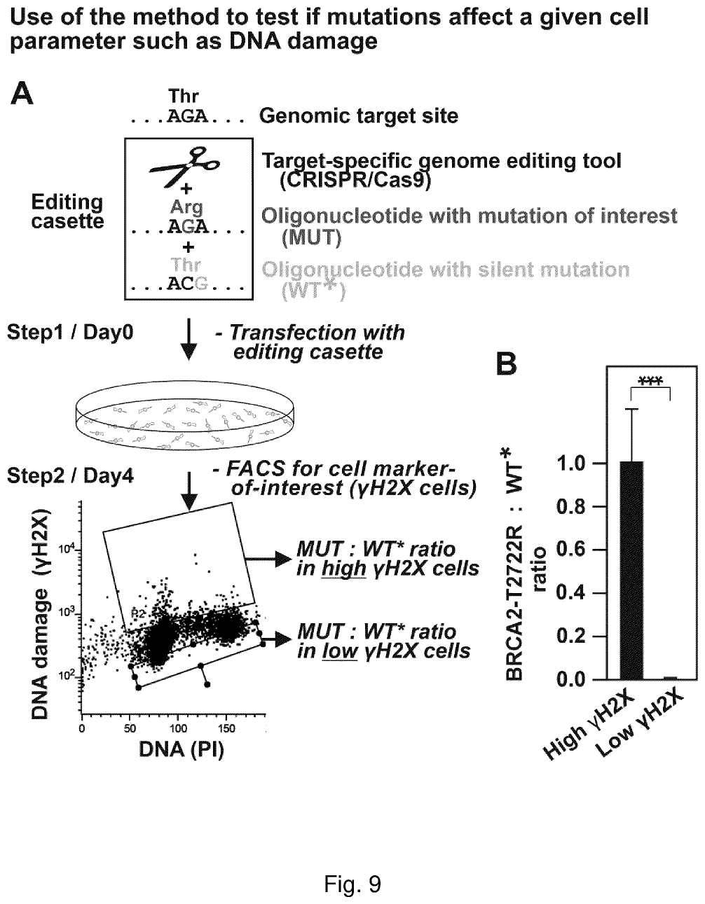

FIG. 9 demonstrates an additional example of the use of the method to test, if a mutation of interest affects a specific (spatial) cell parameter, illustrated with the effect of pathogenic BRCA2 mutations on DNA damage. Specifically, human breast epithelial MCF10A-BRCA2+/− cells were subjected to mutation of BRCA2 with the pathogenic breast and ovarian cancer mutation T2722R and 4 days later analysed by FACS for the DNA damage marker γH2X. Analysis of the T2722R to synonymous WT* mutation ratios in cell subpopulations FACS isolated for either high or low levels of DNA damage showed large enrichment of T2722R in the former population, which thereby demonstrates that pathogenic BRCA2 mutations cause DNA damage. In conclusion, this experiment provides another demonstration that the method can link a specific genetic variant to a specific cell parameter, which can be monitored by FACS.

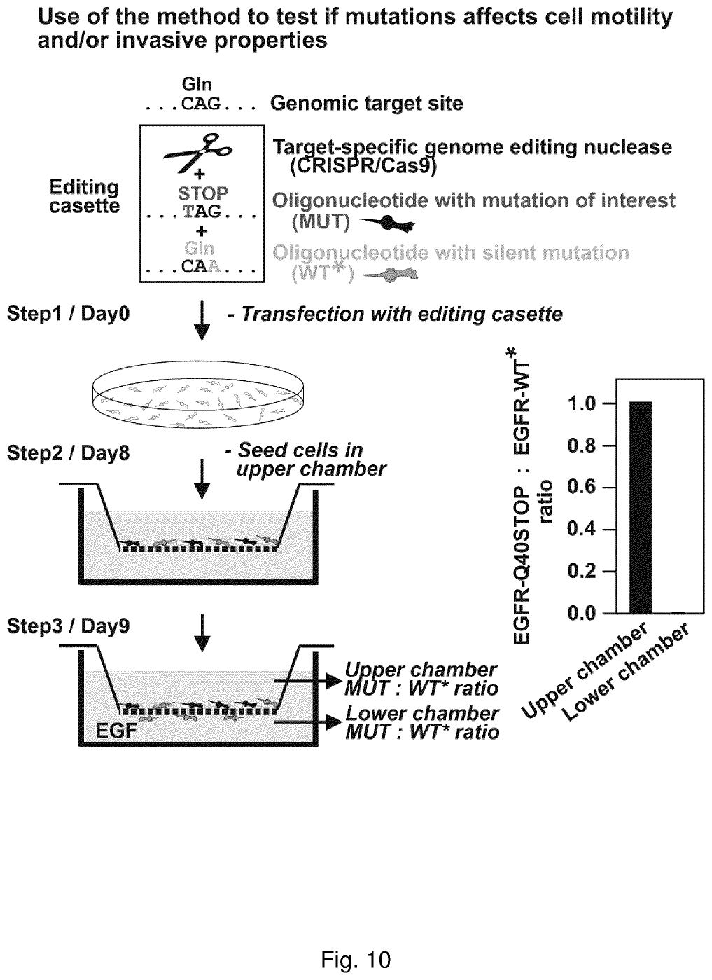

FIG. 10 demonstrates the use of the method to test if mutations affect cell motility and invasive properties. It also provides another example of the use of the method to link a genetic variant to a spatial parameter of cells. Specifically, human breast epithelial MCF10A cells were subjected to mutation of EGFR with the loss-of-function Q40STOP mutation. At Day 8, the cell population was seeded in the upper chamber of an invasion chamber with EGF as chemo-attractant and at Day9, the Q40STOP to synonymous WT* mutation ratios in the cell subpopulations in the upper and lower compartments of the invasion chamber were determined. The results showed virtual absence of the Q40STOP mutation in the lower compartment compared to WT*, which thereby demonstrates that the Q40STOP mutation abrogates cell motile and invasive behavior. In conclusion, this experiment provides a demonstration that the method can link a specific genetic variant to cell motility and invasion.

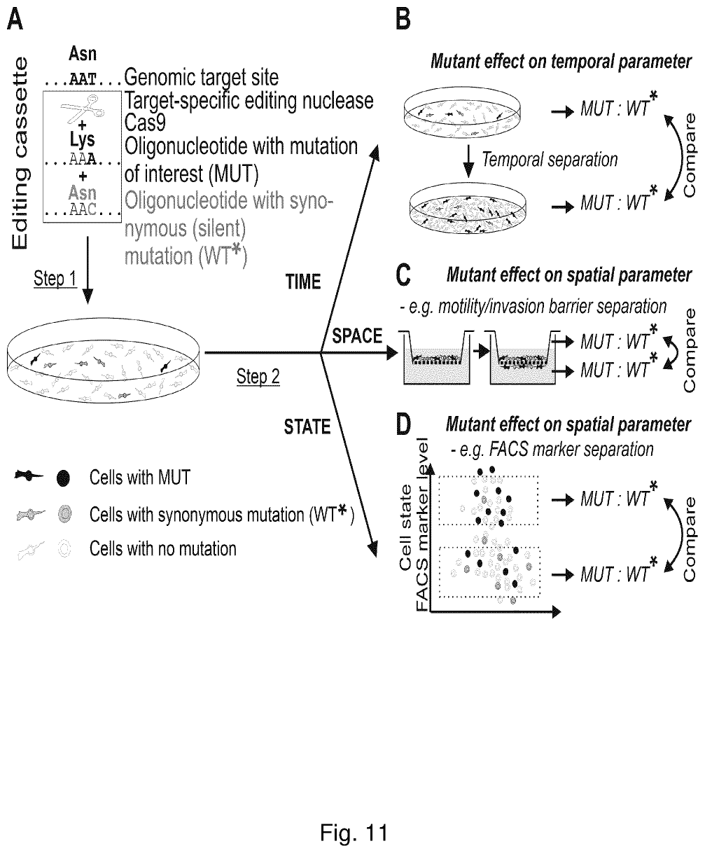

FIG. 11 shows the principle of the method with emphasis on its use as a multiparametric functional analysis of genetic sequence variants by determining mutant:WT* ratios as a function of a temporal parameter or a spatial parameter that can take multiple forms. (A) Step 1, shows that a cell population is transfected with an editing cassette comprising a target-specific genome editing nuclease such as CRISPR-Cas9 and two ssODN repair templates that are identical, except that one harbors the mutation-of-interest (MUT) and the other a synonymous, internal normalization mutation (WT*). Step 2, shows that the ratios of cells with introduction of MUT to WT* are determined as a function of either a temporal parameter (TIME) or a spatial parameter (SPACE) which may be a physically distinct compartment or a cell state parameter (STATE), which is also a spatial parameter that may separate cell populations according to FACS marker levels. (B) for TIME, comparison of MUT:WT* ratios at an early and a subsequent time point determines selection for or against the mutant, which is a readout of the mutant effect on cell proliferation/survival/fitness or similar properties. (C) for SPACE, comparison of MUT:WT* ratios in an initial compartment and a spatially distant compartment determines effect of mutant on cell motile/invasive or similar properties. (D) for STATE, comparison of MUT:WT* ratios in two cell populations FACS separated according to different levels of a marker for a cell state of interest determines the effect of the mutant on that cell state. By using the method with spatial separation by STATE, the method can determine the effect of a variant on any conceivable physiological or pathological state or process of a cell for which a FACS marker is available.

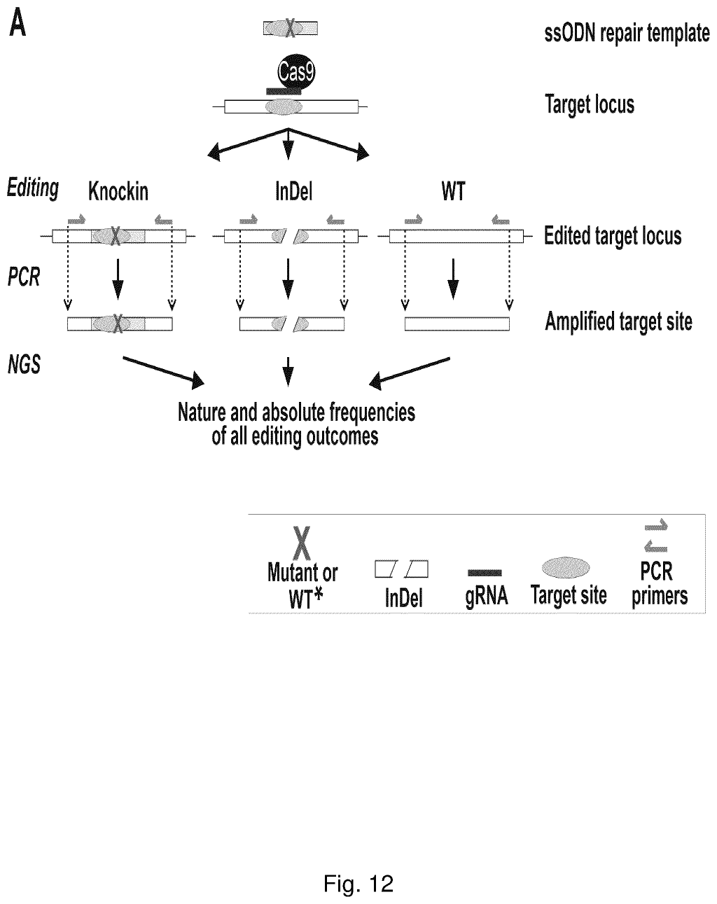

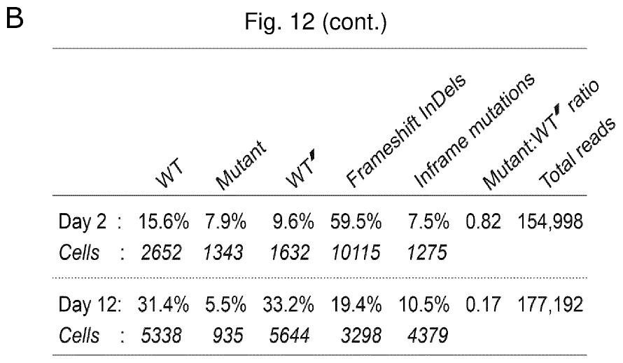

FIG. 12 shows that the specific design of the PCR/NGS target site analysis of the method determines nature and absolute frequencies of all editing outcomes in the cell population, allowing critical controls. (A) Schematic representation of the target site PCR and amplicon NGS analysis, highlighting that the method samples and determines nature and absolute frequencies of all alleles in the cell population, because PCR primers anneal to unmodified sequences outside the region covered by the ssODNs. Thereby, cells (alleles) with introduction (knockin) of the mutation of interest (mutant), the silent synonymous mutation (WT*), InDels as well as WT cells in which no mutation has been introduced are all identified and quantified by the NGS analysis. (B) As an example, a culture of human breast epithelial MCF10A cells (BRCA2+/+) expressing SpCas9 was transfected with synthetic gRNA/tracrRNA targeting a genomic site in BRCA2 overlapping the site to be mutated along with an oligonucleotide comprising the mutation of interest (MUT), which was a known pathogenic variant (T2722R), and an oligonucleotide comprising a synonymous mutation (WT*). At Day 2 after transfection, an aliquot of the cell culture was analysed by PCR amplification of the genomic target site and NGS of the PCR product to determine the ratio of 2722R to WT* alleles in the cell population. Thereafter, cells were cultured until Day 12, where after the mutation analysis was repeated. The table shows the editing outcomes determined at Day 2 and Day 12 by the analysis. Since 100 ng genomic DNA (=approximately 17,000 cells) was used as template for the PCR analysis, the absolute frequencies of the various edited or WT alleles (which approximate cell numbers) present in the cell populations can be calculated. Thereby it can be controlled that results are based on statistically sufficient cell numbers.

FIG. 13 shows that the method contains “built-in loss-of-heterozygosity” to reveal effects of loss-of-function mutations in tumor suppressor genes in diploid cells. (A) As an example, a culture of human breast epithelial MCF10A cells (BRCA2+/+) expressing SpCas9 was transfected with synthetic gRNA/tracrRNA targeting a genomic site in BRCA2 overlapping the site to be mutated along with an oligonucleotide comprising the mutation of interest (MUT), which was a known pathogenic variant (T2722R), and an oligonucleotide comprising a synonymous mutation (WT*). At Day 2 after transfection, an aliquot of the cell culture was analysed by PCR amplification of the genomic target site and NGS of the PCR product to determine the ratio of 2722R to WT* alleles in the cell population. Thereafter, cells were cultured until Day 12, where after the mutation analysis was repeated. The robust detection of loss-of-function effect of the T2722R mutation in the tumor suppressor BRCA2 suggests that cells with knockin of the T2722R mutation on one allele had a BRCA2 inactivating editing outcome on the other allele. (B) To test if such a scenario takes place (as it has not been previously demonstrated in the literature), single cells from the experiment in (A) were FACS sorted on Day 2 and analysed by amplicon Sanger sequencing of the BRCA2-T2722 target site to determine the editing outcomes on both alleles of individual cells. The results demonstrated that the large majority of cells with knockin of the T2722R mutation-of-interest on one allele had the other allele inactivated by mostly frameshift InDels, or alternatively another T2722R mutation, thereby effectively exhibiting “built-in loss-of-heterozygosity”. (C) shows a schematic illustration of the “loss-of-heterozygosity” feature inherent to the method.

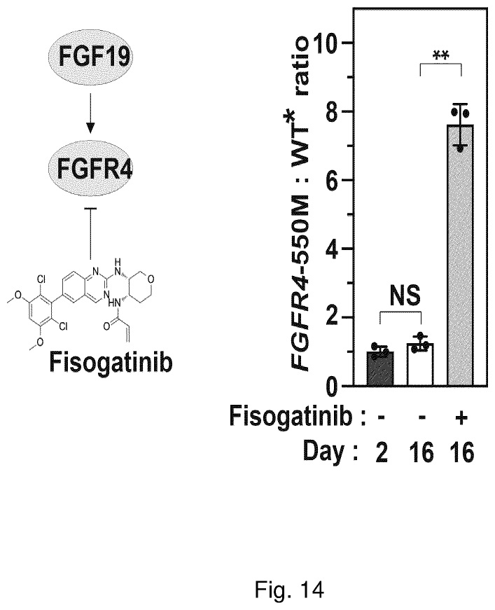

FIG. 14 shows use of the method to determine mechanisms of drug resistance and that drugs act on-target to inhibit cancer cells, demonstrated by the mutation V550M in the receptor gene FGFR4, which causes resistance to the selective FGFR4 inhibitor Fisogatinib, a drug for liver cancers overexpressing the growth factor FGF19. Cultures of human liver cancer Hep3B cells, which harbor FGF19 focal amplification, were nucleofected with SpCas9 protein and synthetic gRNA/tracrRNA targeting a genomic site overlapping the site to be mutated along with an oligonucleotide comprising the mutation of interest (V550M) in the Fisogatinib-binding site of FGFR4 and an oligonucleotide comprising a synonymous mutation (WT*). At Day 2 after nucleofection, an aliquot of the cell culture was analysed by PCR amplification of the genomic target site and NGS of the PCR product to determine the ratio of 550M to WT* alleles in the cell population. Thereafter, cells were split in two cultures, and cultured in the absence (−) or presence (+) of 0.72 μM Fisogatinib until Day 16, whereafter the mutation analysis was repeated on each culture. As can be seen from the results, cells harboring the FGFR4-V550M mutation were strongly selected for, when treated with Fisogatinib. In conclusion, the method can thus determine 1) that the V550M mutation confers resistance to Fisogatinib and 2) that this drugs acts via its intended FGFR4 target to inhibit the cancer cells. Such applications of the method are important for clinical management as well as for drug development in the pharmaceutical industry.

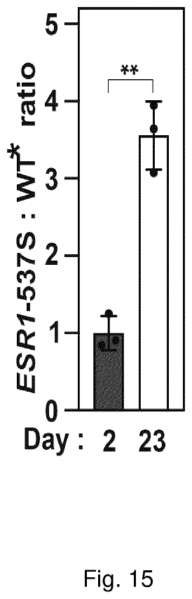

FIG. 15 shows the use of the method to determine mechanisms of drug resistance demonstrated by the oncogenic mutation Y537S in the estrogen receptor gene ESR1, the major resistance mechanism to the estrogen antagonist Tamoxifen, the mainstay drug for estrogen receptor-positive breast cancers. Cultures of human breast cancer MCF7 cells were nucleofected with SpCas9 protein and synthetic gRNA/tracrRNA targeting a genomic site overlapping the site to be mutated along with an oligonucleotide comprising the oncogenic mutation of interest (Y537S) in ESR1 and an oligonucleotide comprising a synonymous mutation (WT*). At Day 2 after nucleofection, an aliquot of the cell culture was analysed by PCR amplification of the genomic target site and NGS of the PCR product to determine the ratio of 537S to WT* alleles in the cell population. Thereafter, cells were cultured under serum-, growth factor- and phenol red-deprived conditions (i.e. estrogen-free conditions) until Day 23, where after the mutation analysis was repeated. The results demonstrate that the Y537S mutation confers estrogen-independent proliferation of the MCF7 breast cancer cells, providing a mechanism for Tamoxifen resistance. In conclusion, this represents a further example of the use of the method to determine mechanisms of drug resistance.

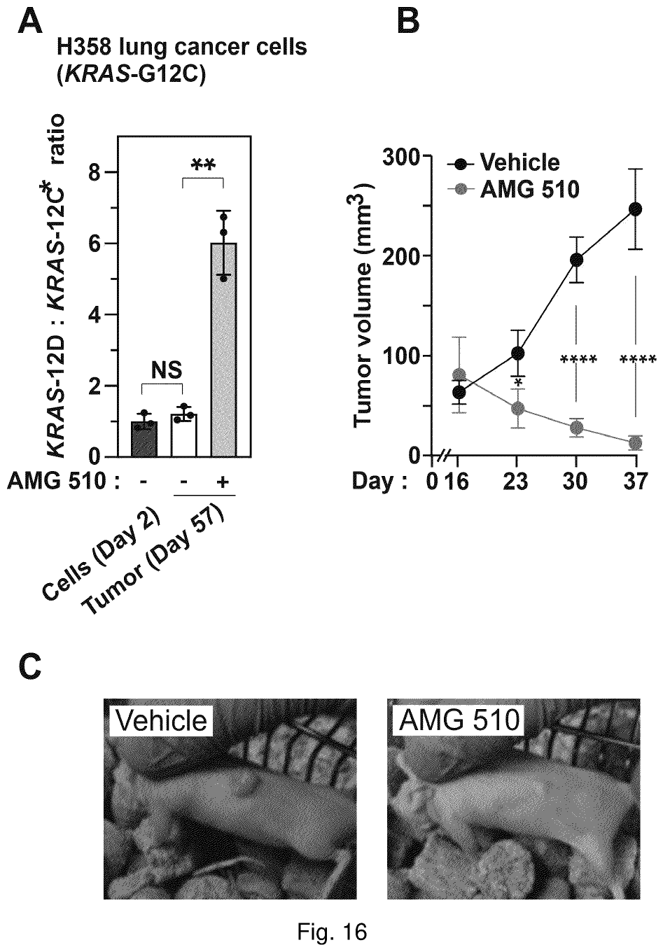

FIG. 16 shows in vivo use of the method to determine mechanisms of drug resistance and that drugs act on-target to inhibit tumor growth, demonstrated using the oncogenic KRAS-G12C mutant covalent inhibitor AMG 510 (for inhibitor, see FIG. 7). A culture of human lung cancer H358 cells was nucleofected with SpCas9 protein and synthetic gRNA/tracrRNA targeting the oncogenic 12C mutation in KRAS present in these cells along with an oligonucleotide comprising the mutation of interest, which was another oncogenic KRAS mutation (12D), and an oligonucleotide comprising a synonymous mutation (12C*). At Day 2 after transfection, an aliquot of the cell culture was analysed by PCR amplification of the genomic target site and NGS of the PCR product to determine the ratio of 12D versus 12C* alleles in the cell population. Next, the cells were xenografted into 2 nude mice and two weeks after, either mouse was treated once per day with vehicle (−) or AMG 510 (+). (A) On day 57, the mutation analysis was repeated on the resultant tumors to determine the selection effect of AMG 510 on KRAS-12C* versus KRAS-12D variants in the tumors. (B) Tumor volumes were determined over time and, (C) representative mice photographed on day 40. As can be seen from the results, while AMG 510 greatly reduced bulk tumor size, within the tumors, cells harboring the KRAS-12D mutation were strongly selected for by the AMG 510 treatment, demonstrating that the 12D mutation confers resistance to this compound. In conclusion, the method can be used for in vivo determination of mechanisms of drug resistance and determination that drugs act on-target to inhibit tumor growth.

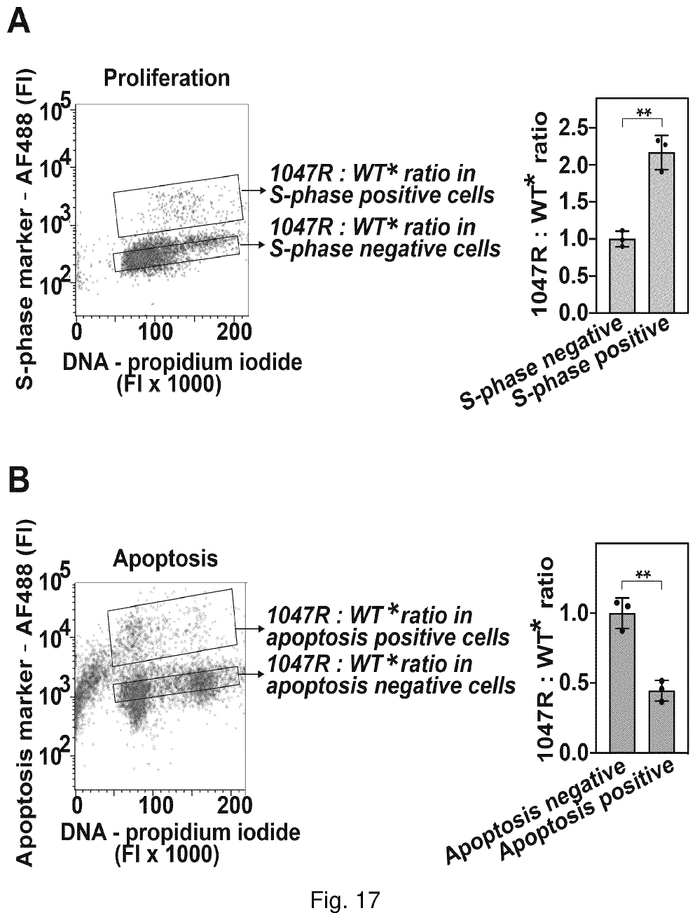

FIG. 17 shows use of the method to test if a mutation affects a given cell parameter of interest, such as DNA replication or apoptosis by determining mutant:WT* ratios in cell populations spatially separated by FACS according to markers for these parameters. Cultures of human breast epithelial MCF10A cells expressing SpCas9 were transfected with synthetic gRNA/tracrRNA targeting a genomic site overlapping the site to be mutated along with an oligonucleotide comprising the oncogenic mutation of interest (H1047R) in PIK3CA and an oligonucleotide comprising a synonymous mutation (WT*). At Day 3 after transfection, cells were shifted to serum- and growth factor-depleted medium. On day 7, cells were either (A) pulsed for 2 h with S-phase marker EdU and stained for EdU or (B) labelled with TUNEL apoptosis marker BrdU and stained for BrdU. Next, cells were FACS isolated according to being (A) either positive or negative for EdU or (B) either positive or negative for BrdU. Each population isolated in (A) and (B) was analysed by PCR amplification of the genomic target site and NGS of the PCR product to determine the ratio of 1047R to WT* alleles. Data represent ratios normalized to the value in the (A) S-phase (EdU) negative population or (B) apoptosis (BrdU) negative population. As can be seen from the results, the PIK3CA-1047R mutation was enriched in the S-phase positive population relative to the S-phase negative cell population (A), demonstrating that the variant stimulated proliferation of the cells. Furthermore, the PIK3CA-1047R mutation was enriched in the apoptosis-negative cell population, demonstrating that the variant conferred resistance to apoptosis (B). In conclusion, by using the method with spatial separation of cells according to the level of a FACS marker, the method can determine the effect of a variant on any conceivable physiological or pathological state or process of a cell for which a FACS marker is available.

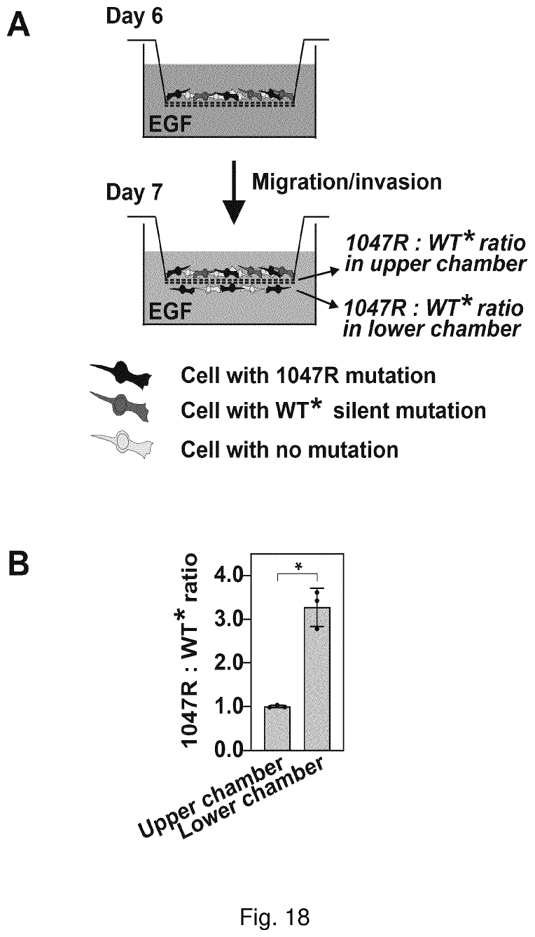

FIG. 18 shows use of the method to test if an oncogenic mutation affects a given cell parameter of interest such as stimulation of cell migration/invasion by determining mutant:WT* ratios in cell populations spatially separated by a motility/invasion barrier. Specifically, cultures of human breast epithelial MCF10A cells expressing SpCas9 were transfected with synthetic gRNA/tracrRNA targeting a genomic site overlapping the site to be mutated along with an oligonucleotide comprising the mutation of interest (H1047R) in PIK3CA and an oligonucleotide comprising a synonymous mutation (WT*).

At Day 3 after transfection, the cells were shifted to serum- and growth factor-depleted medium. (A) on Day 6, the cells were seeded on a Matrigel-coated, cell-permeable membrane in the upper chamber of a Transwell invasion chamber with serum- and growth factor-depleted medium, except that EGF was added to the lower chamber as chemo-attractant, and the cells were allowed to migrate/invade for 16 h. (B) at Day 7, the cells in the upper and lower chambers were analysed by PCR amplification of the genomic target site and NGS of the PCR product to determine the ratio of 1047R to WT* in the two cell populations. As can be seen from the results, the PIK3CA-1047R mutant was enriched in the lower chamber relative to the upper chamber, thereby demonstrating that the mutation stimulated the migrative and/or invasive properties of the cells. In conclusion, by using the method with spatial separation of cells according an initial compartment and a spatially distant compartment, the method can determine effects of a mutant on cell motile/invasive or similar properties.

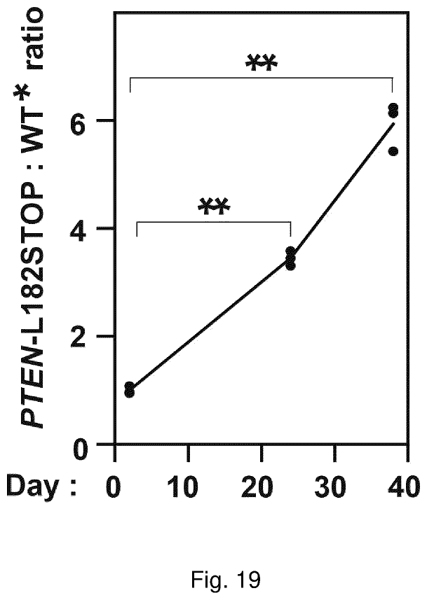

FIG. 19 shows use of the method to test if a putative loss-of-function (i.e. a candidate driver) mutation in a tumor suppressor gene increases cell proliferation/survival consistent with it being a bona-fide tumor suppressor gene. The figure also shows that the “built-in loss-of-heterozygosity” feature of the method works robustly. A culture of human breast epithelial MCF10A (PTEN+/+) cells expressing SpCas9 were transfected with synthetic gRNA/tracrRNA targeting a genomic site overlapping the site to be mutated along with an oligonucleotide comprising the loss-of-function mutation of interest (L182STOP) in the tumor suppressor PTEN and an oligonucleotide comprising a synonymous mutation (WT*). At Day 2 after transfection, an aliquot of the cell culture was analysed by PCR amplification of the genomic target site and NGS of the PCR product to determine the ratio of 182STOP to WT* alleles in the cell population. Next, the cells were cultured under serum- and growth factor-deprived conditions for up to 38 days, with determination of 182STOP to WT* ratios at the indicated time points. As can be seen from the results, the method determined selection for cells with the PTEN-L182STOP mutation, which accumulated over time, in accordance with the established driver function of this variant. In conclusion, the method can accurately determine driver role of loss-of-function mutations in suppressor genes. Furthermore, since the cells were PTEN+/+, the “built-in loss-of-heterozygosity” feature of the method works robustly to allow such demonstration of loss-of-function mutations in tumor suppressor genes in diploid cells.

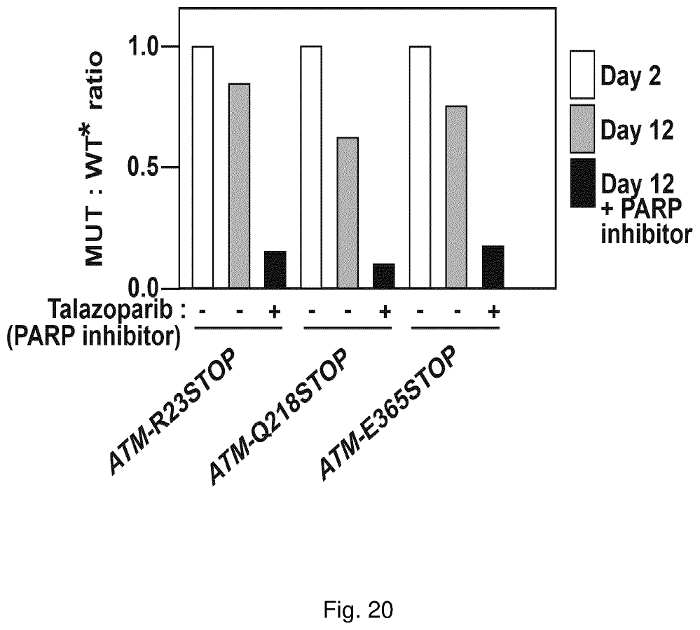

FIG. 20 shows yet another example of the use of the method to categorize cancer-associated variants as pathogenic and to predict their PARP-inhibitor responsiveness, illustrated with the ATM gene, which underlies hereditary breast and ovarian cancer. The figure also shows yet another example that the “built-in loss-of-heterozygosity” feature of the method works robustly. Cultures of human breast epithelial MCF10A (ATM+/+) cells expressing SpCas9 were transfected with synthetic gRNA/tracrRNA targeting genomic sites in ATM overlapping the sites to be mutated along with an oligonucleotide comprising the mutation of interest (MUT), which was as known loss-of-function variants (R23STOP, Q218STOP or E365STOP), and an oligonucleotide comprising a corresponding synonymous mutation (WT*). At Day 2 after transfection, an aliquot of each culture was analysed by PCR amplification of the genomic target site and NGS of the PCR product to determine the ratio of MUT to WT* alleles in the cell population. Thereafter, cells were cultured in the absence (−) or presence (+) of 2 nM Talazoparib (a PARP inhibitor) until Day 12, where after the mutation analysis was repeated. As can be seen from the results, the loss-of-function ATM mutations caused loss of viability in the cells and sensitized them to PARP inhibitor killing. In conclusion, the method can correctly categorize cancer-associated variants as pathogenic and to predict their drug responsiveness, which can be directly used for molecular genetics diagnosis in the clinic as well as for drug development in the pharmaceutical industry. Furthermore, since the cells were ATM+/+, the “built-in loss-of-heterozygosity” feature of the method works robustly to allow such demonstration of loss-of-function mutations in tumor suppressor genes in diploid cells.

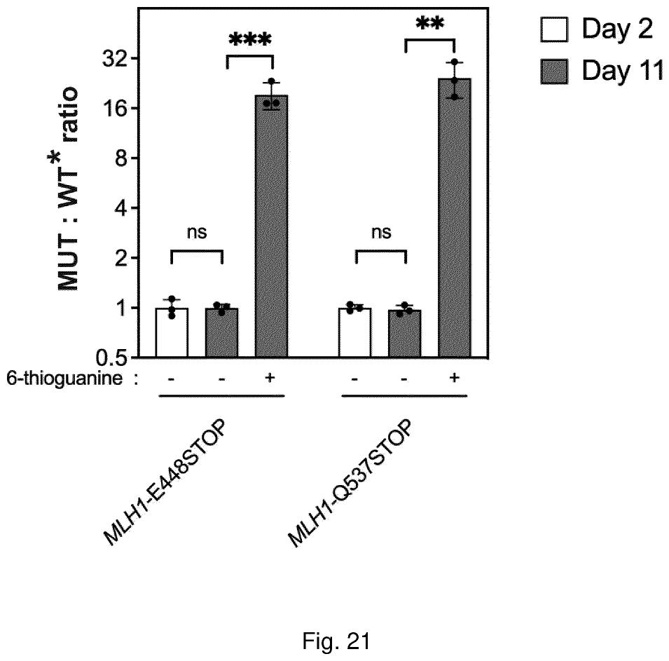

FIG. 21 shows yet another example of the use of the method to catagorize cancer-associated variants as pathogenic, using the MLH1 gene as an example, which underlies hereditary colorectal cancer (Lynch syndrome). The figure also shows yet another example that the “built-in loss-of-heterozygosity” feature of the method works robustly. Cultures of human colon cancer SW620 (MLH1+/+) cells were nucleofected with SpCas9 protein and synthetic gRNA/tracrRNA targeting genomic sites in MLH1 overlapping the sites to be mutated along with an oligonucleotide comprising the mutation of interest (MUT), which was as a known loss-of-function variant (E448STOP or Q537STOP), and an oligonucleotide comprising a corresponding synonymous mutation (WT*). At Day 2 after nucleofection, an aliquot of the cell culture was analysed by PCR amplification of the genomic target site and NGS of the PCR product to determine the ratio of MUT to WT* alleles in the cell population. Thereafter, cells were split in two cultures, and cultured in the absence (−) or presence (+) of 2 μM 6-thioguanine until Day 11, where after the mutation analysis was repeated for each culture. As can be seen from the results, the loss-of-function mutations disabled the ability of MLH1 to elicit apoptosis in response to 6-thioguanine, as expected. In conclusion, the method can correctly categorize cancer-associated variants in colorectal cancer predisposition genes as pathogenic, which can be directly used for molecular genetics diagnosis in the clinic. Furthermore, since the cells were MLH1+/+, the “built-in loss-of-heterozygosity” feature of the method works robustly to allow such demonstration of loss-of-function mutations in tumor suppressor genes in diploid cells.

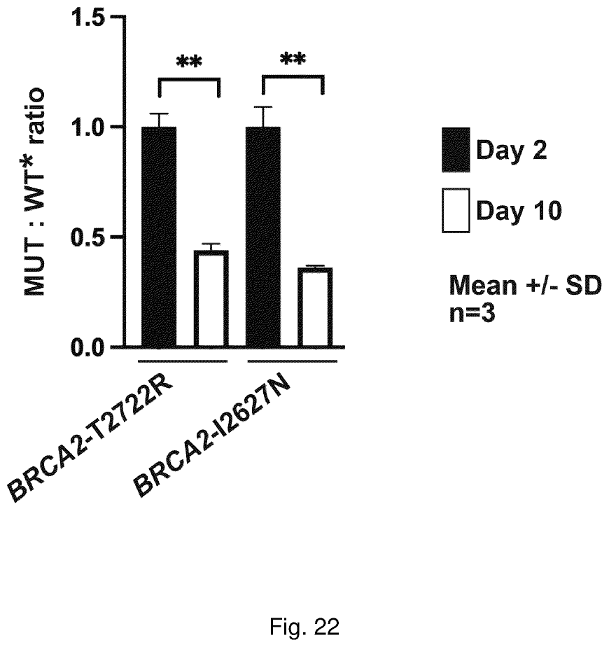

FIG. 22 shows that the method also works with NG-SpCas9. Cultures of human breast epithelial MCF10A cells expressing NG-SpCas9 were transfected with synthetic gRNA/tracrRNA targeting genomic sites in BRCA2 overlapping the sites to be mutated along with an oligonucleotide comprising the mutation of interest (MUT), which was as a known pathogenic variant (T2722R or I2627N), and an oligonucleotide comprising a corresponding synonymous mutation (WT*). At Day 2 after transfection, an aliquot of each culture was analysed by PCR amplification of the genomic target site and NGS of the PCR product to determine the ratio of MUT to WT* alleles in the cell population. This analysis was repeated on Day 10. As can be seen from the results, the assay worked well, showing that the pathogenic variants caused cell loss-of-viability. In conclusion, the method works well with NG-SpCas9.

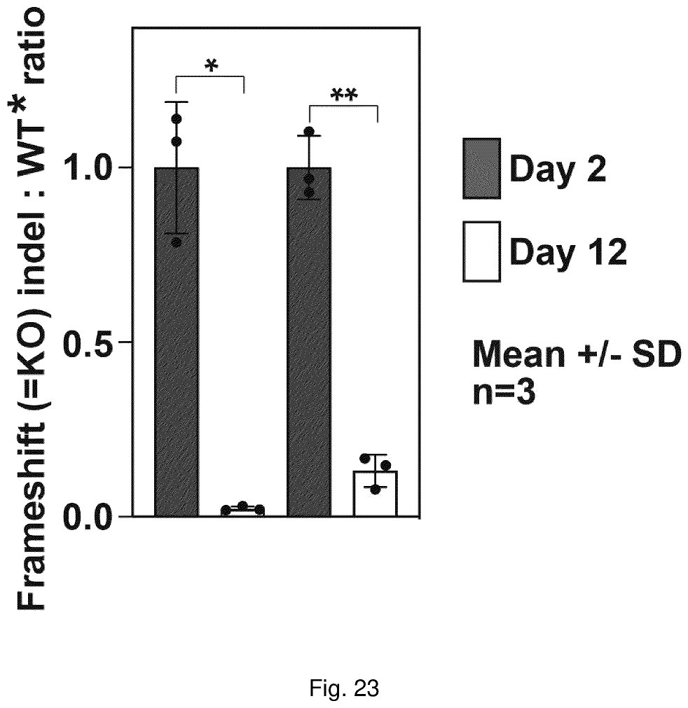

FIG. 23 illustrates that the method contains an internal frameshift (=knockout) indel control, which can be normalized against WT* and which can show that apparently neutral variants are truly neutral. Thus, in addition to cells with knockin of the mutation of interest or the WT* synonymous mutation, a substantial fraction of cells will have introduction of a frameshifting indel (insertion/deletion) mutation, which are loss-of-function mutations (knockout=KO). The fate of these cells over time can also be analysed in the same cell culture, where the neutral variant was investigated. As shown in FIG. 23, in the same MCF10A-BRCA2+/− cell culture, where the BRCA2 mutations N289H or D946V did not reduce proliferation and/or survival between the initial (Day 2) and subsequent (Day12), i.e. they behaved as neutral variants (see FIG. 4A), the cells with frameshifting indels were strongly selected against, as evidenced from the decrease of the ratio of frameshifting indel knockout (KO) mutations to WT* alleles in the cell population over time. In conclusion, the internal indel control demonstrates that the assay worked during this experiment, meaning that the lack of effect of the N289H and D946V mutations is because they are truly neutral. In addition, the usefulness of the WT* synonymous mutation is extended here and shown to also serve as an internal wild-type normalization control for the frequency of frameshift indel mutations, making the frameshift control for neutral variants very reliable.

Sequence overview

| SEQ | | Organism and optionally accession |
|---|---|---|
| ID NO: | Description | number |
| 1 | Cas9 (protein) | Streptococcus pyogenes, (Q99ZW2) |
| 2 | Human, codon- | Sequence is in silico translation of Cas9- |
| | optimized (D10A) | D10A in the plasmid pCas9n-sgPdx1 |
| | nickase (protein) | (AMQ45845) |
| 3 | Cas12a (protein) | Francisella tularensis, subspecies novicida |
| | | (A0Q7Q2) |
| 4 | Cas9-NG (DNA) | Sequence derived from Cas9-NG in the |
| | | plasmid pX330-SpCas9-NG |
| 5 | Cas9-NG | In silico translation based on DNA sequence |
| | (protein) | of Cas9-NG in the plasmid pX330-SpCas9- |
| | | NG (Addgene #117919) |
| 6 | Cas9 (DNA) | Streptococcus pyogenes |
| 7 | Human, codon- | Sequence is Cas9-D10A in the plasmid |
| | optimized (D10A) | pCas9n-sgPdx1 (AMQ45845) |
| | nickase (DNA) | |
| 8 | Cas12a (DNA) | Francisella novicida |
| 9 | PE2 (protein) | Artificial |
| 10 | PE2 (DNA) | Artificial |

### REFERENCES

Anzalone, Andrew V., et al. “Search-and-replace genome editing without double-strand breaks or donor DNA.” Nature 576.7785 (2019): 149-157.
Carroll D, Morton J J, Beumer K J, Segal D J (2006). “Design, construction and in vitro testing of zinc finger nucleases” Nature protocols, 1:1329-1341.
Sanjana N E, Cong L, Zhou Y, Cunniff M M, Feng G, Zhang F (2012). “A transcription activator-like effector toolbox for genome engineering” Nature protocols, 7:171-192.
Silva G, Poirot L, Galetto R, Smith J, Montoya G, Duchateau P, Paques F (2011). “Meganucleases and other tools for targeted genome engineering: perspectives and challenges for gene therapy” Current gene therapy, 11:11-27.
Wright D A, Thibodeau-Beganny S, Sander J D, Winfrey R J, Hirsh A S, Eichtinger M, Fu F, Porteus M H, Dobbs D, Voytas D F, Joung J K (2006). “Standardized reagents and protocols for engineering zinc finger nucleases by modular assembly” Nature protocols, 1:1637-1652.

Items

1. A method for assessing the effects of a mutation of interest in a cell, said method comprising the steps of:
i) providing a cell population comprising a target nucleic acid sequence;
ii) introducing in at least some of the cells of the cell population:
   a) a nuclease or a polynucleotide encoding said nuclease, wherein the nuclease is capable of generating one or more single-strand breaks (SSBs) or double-strand breaks (DSBs) in a target nucleic acid sequence, and targeting means directing the nuclease to the target nucleic acid sequence, whereby the nuclease is capable of binding to a binding region of the target nucleic acid sequence, and whereby the nuclease is capable of generating one or more single-stranded breaks (SSBs) or double-strand breaks (DSBs) in the target nucleic acid sequence;
   b) a first oligonucleotide comprising a mutation of interest, preferably a non-silent mutation, preferably a non-synonymous mutation, and otherwise identical to or complementary to said target nucleic acid sequence, wherein said mutation of interest preferably lies within the binding region of said nuclease; and
   c) a second oligonucleotide comprising a synonymous mutation, wherein said synonymous mutation preferably lies within the binding region of said nuclease;
   whereby said first oligonucleotide or said second oligonucleotide is integrated in, or copied into, the target nucleic acid sequence of at least some of the cells, thereby obtaining a mixed population of cells comprising cells in which the mutation of the first or the second oligonucleotides has not been introduced, cells in which only the mutation of the first oligonucleotide has been introduced, and cells in which only the mutation of the second oligonucleotide has been introduced;
iii) incubating the mixed population of cells in a medium for a determined duration, under conditions allowing a parameter of interest to be monitored, wherein the parameter of interest is a temporal parameter and/or a spatial parameter;
iv) determining the effect of the mutation of interest on the parameter of interest, wherein:
   A. if the parameter of interest is a temporal parameter, determining the effect of the mutation of interest on the parameter of interest comprises the steps of:
      v) determining an initial ratio of cells in which the mutation of interest has been introduced in the target nucleic acid sequence to cells in which the synonymous mutation has been introduced in the target nucleic acid sequence, wherein the initial ratio of cells is determined at an initial time point; determining a subsequent ratio of cells in which the mutation of interest has been introduced in the target nucleic acid sequence to cells in which the synonymous mutation has been introduced in the target nucleic acid sequence, wherein the subsequent ratio of cells is determined at a subsequent time point; and determining a change in ratio between the initial ratio and the subsequent ratio; and
      vi) correlating said change in ratio to the parameter of interest, and/or
   B. if the parameter of interest is a spatial parameter, determining the effect of the mutation of interest on the parameter of interest comprises the steps of:
      v) defining and/or spatially separating subpopulations of cells on the basis of said spatial parameter of interest in each subpopulation, preferably wherein the spatial parameter of interest is different in each subpopulation;
      vi) determining, for each subpopulation, a ratio of cells in which the mutation of interest has been introduced in the target nucleic acid sequence to cells in which the synonymous mutation has been introduced in the target nucleic acid sequence; and
      vii) correlating said ratio to the measured spatial parameter of interest for each subpopulation;
         thereby assessing the effect of the mutation on the parameter of interest.

2. The method according to item 1, wherein step A.v) further comprises a step of determining an initial frequency of cells with an indel in the target nucleic acid sequence which is different from the mutation of the first and the second oligonucleotides, wherein the initial frequency of cells is determined at said initial time point, and determining a subsequent frequency of cells with an indel in the target nucleic acid sequence which is different from the mutation of the first and the second oligonucleotides, wherein the subsequent frequency of cells is determined at said subsequent time point,
or wherein step B.vi) further comprises a step of, for each subpopulation, determining a frequency of cells with an indel in the target nucleic acid sequence which is different from the mutation of the first and the second oligonucleotides.

3. The method according to item 2, wherein the initial and the subsequent frequency of cells with an indel is further subdivided into, respectively, an initial and a subsequent frequency of cells with an indel resulting in a frameshift mutation and an initial and a subsequent frequency of cells with an indel not resulting in a frameshift mutation, wherein a subsequent frequency of cells with an indel resulting in frameshift mutation lower or higher than the initial frequency of cells with an indel resulting in frameshift mutation indicates that the frameshift indels are affecting the cells.

4. The method according to any one of the preceding items, wherein said first oligonucleotide consists of or comprises a stretch of nucleotides identical to said second oligonucleotide except for said mutation of interest or wherein said second oligonucleotide consists of or comprises a stretch of nucleotides identical to said first oligonucleotide except for said synonymous mutation, or wherein the first oligonucleotide and the second oligonucleotide differ only in the location of the mutation of interest,
and/or wherein the synonymous mutation is a single base change compared to the genomic target region and/or wherein the mutation of interest is a single base change compared to the genomic target region.

5. The method according to any one of items 1 to 3, wherein the synonymous mutation of said second oligonucleotide is located in a different position from the mutation of interest in said first oligonucleotide.

6. The method according to any one of the preceding items, wherein the parameter of interest is a temporal parameter of interest, and wherein:
A. If the initial ratio of cells is lower than the subsequent ratio of cells, the mutation is characterised as a mutation having a positive effect on the temporal parameter;
B. If the initial ratio of cells is greater than the subsequent ratio of cells, the mutation is characterised as a mutation having a negative effect on the temporal parameter;
C. If the initial and the subsequent ratio of the cells are substantially the same, the mutation is characterised as a mutation having no effect on the temporal parameter.

7. The method according to any one of the preceding items, wherein step B.v) comprises defining at least one subpopulation of cells and a reference subpopulation of cells from the mixed population on the basis of the spatial parameter of interest and wherein:
A. If the ratio of cells in said subpopulation is greater than in said reference subpopulation, the mutation is characterised as a mutation having a positive effect on the spatial parameter;
B. If the ratio of cells in said subpopulation is lower than the ratio of said reference subpopulation, the mutation is characterised as a mutation having a negative effect on the spatial parameter;
C. If the ratio of cells in said subpopulation is substantially the same as the ratio of cells in said reference subpopulation, the mutation is characterised as a mutation having no effect on the spatial parameter,
   optionally, wherein step B.v) further comprises spatially separating said at least one subpopulation from the reference subpopulation.

8. The method according to any one of the preceding items, wherein step B.v) comprises a step of spatially separating the cells to separate the mixed cell population into subpopulations on the basis of a cell marker, such as the presence or absence or a graded level of a cell marker, for example a cell marker for cell proliferation, cell growth, fitness, cell motility, cell invasiveness, cellular metabolism, cell differentiation, DNA damage, expression levels of pre-defined genes or proteins, resistance to a compound, sensitivity to a compound, production of a compound, anoikis, senescence, apoptosis, DNA methylation or protein post-translational modification, optionally wherein the cell marker is the spatial parameter of interest,
or wherein step B.v) comprises defining the subpopulations on the basis of a cell property, such as the ability of the cells to migrate or invade, optionally wherein the cell property is the spatial parameter of interest.

9. The method according to any one of the preceding items, wherein the temporal parameter is selected from the group consisting of cell proliferation, cell growth, anchorage-independent cell growth, contribution to tumor growth, fitness, cell motility, cell invasiveness, cellular metabolism, DNA damage, expression levels of pre-defined genes and/or proteins, resistance to a compound, sensitivity to a compound, production of a compound, anoikis, senescence, contact inhibition and apoptosis.
or wherein the spatial parameter is selected from the group consisting of: cell proliferation, cell growth, fitness, cell motility, cell invasiveness, cellular metabolism, cell differentiation, DNA damage, expression levels of pre-defined genes and/or proteins, resistance to a compound, sensitivity to a compound, production of a compound, anoikis, senescence, apoptosis, DNA methylation, and protein post-translational modification.

10. The method according to any one of the preceding items, wherein the first oligonucleotide comprises at least one mutation of interest within the binding region of the nuclease, and wherein the second oligonucleotide comprises at least one synonymous mutation within the same binding region of the nuclease.

11. The method according to any one of the preceding items, wherein the first oligonucleotide is a plurality of first oligonucleotides each comprising a pre-determined mutation of interest and wherein the second oligonucleotide is a plurality of second oligonucleotides each comprising a pre-determined synonymous mutation.

12. The method according to any one of the preceding items, wherein the nuclease comprises or consists of a CRISPR/Cas nuclease and the targeting means comprise or consist of a guide RNA capable of hybridizing to the genomic target region.

13. The method according to any one of items 1 to 11, wherein the nuclease and the targeting means are selected from the group consisting of: a transcription activator-like effector nuclease (TALEN) consisting of a DNA cleavage domain and a DNA-binding domain; a zinc finger nuclease consisting of a DNA cleavage domain and a DNA-binding domain; and a meganuclease.

14. A system comprising:
A. a nuclease or a polynucleotide encoding said nuclease, wherein the nuclease is capable of generating one or more single-strand breaks (SSBs) or double-strand breaks (DSBs) in a target nucleic acid sequence, and targeting means directing the nuclease to the target nucleic acid sequence, whereby the nuclease is capable of binding to a binding region of the target nucleic acid sequence, and whereby the nuclease is capable of generating one or more single-stranded breaks (SSBs) or double-strand breaks (DSBs) in the target nucleic acid sequence;
B. a first oligonucleotide comprising a mutation of interest, preferably a non-silent mutation, preferably a non-synonymous mutation, and otherwise identical to or complementary to said target nucleic acid sequence, wherein said mutation of interest preferably lies within the binding region of said nuclease; and
C. a second oligonucleotide comprising a synonymous mutation, wherein said synonymous mutation preferably lies within the binding region of said nuclease.

15. The system according to item 14, wherein the nuclease and the targeting means are selected from the group consisting of a transcription activator-like effector nuclease (TALEN) consisting of a DNA cleavage domain and a DNA-binding domain, a zinc finger nuclease consisting of a DNA cleavage domain and a DNA-binding domain and a meganuclease.

16. A host cell comprising the system according to item 14.

17. Use of the system according to item 14 in a method for assessing the effects of a mutation of interest in a cell, preferably wherein the method is according to any one of items 1 to 13.

1. A method for assessing the effects of a mutation of interest in a cell, said method comprising the steps of:
i) providing a cell population comprising a target nucleic acid sequence;
ii) introducing in at least some of the cells of the cell population:
a) a nuclease or a polynucleotide encoding said nuclease, wherein the nuclease is capable of generating one or more single-strand breaks (SSBs) or double-strand breaks (DSBs) in a target nucleic acid sequence, and targeting means directing the nuclease to the target nucleic acid sequence, whereby the nuclease is capable of binding to a binding region of the target nucleic acid sequence, and whereby the nuclease is capable of generating one or more single-stranded breaks (SSBs) or double-strand breaks (DSBs) in the target nucleic acid sequence;
b) a first oligonucleotide comprising a mutation of interest, preferably a non-silent mutation, preferably a non-synonymous mutation, wherein said non-synonymous mutation introduces a change in the encoded amino acid sequence compared to the amino acid sequence encoded by the target nucleic acid sequence, and otherwise identical to or complementary to said target nucleic acid sequence, wherein said mutation of interest preferably lies within the binding region of said nuclease; and
c) a second oligonucleotide comprising a synonymous mutation, wherein said synonymous mutation preferably lies within the binding region of said nuclease, and wherein said synonymous mutation introduces no change in the encoded amino acid sequence compared to the amino acid sequence encoded by the target nucleic acid sequence;
whereby said first oligonucleotide or said second oligonucleotide is integrated in, or copied into, the target nucleic acid sequence of at least some of the cells, thereby obtaining a mixed population of cells comprising cells in which the mutation of the first or the second oligonucleotides has not been introduced, cells in which only the mutation of the first oligonucleotide has been introduced, and cells in which only the mutation of the second oligonucleotide has been introduced;
iii) incubating the mixed population of cells in a medium for a determined duration, under conditions allowing a parameter of interest to be monitored, wherein the parameter of interest is a temporal parameter and/or a spatial parameter;
iv) determining the effect of the mutation of interest on the parameter of interest, wherein:
A. if the parameter of interest is a temporal parameter, determining the effect of the mutation of interest on the parameter of interest comprises the steps of:
v) determining an initial ratio of cells in which the mutation of interest has been introduced in the target nucleic acid sequence to cells in which the synonymous mutation has been introduced in the target nucleic acid sequence, wherein the initial ratio of cells is determined at an initial time point; determining a subsequent ratio of cells in which the mutation of interest has been introduced in the target nucleic acid sequence to cells in which the synonymous mutation has been introduced in the target nucleic acid sequence, wherein the subsequent ratio of cells is determined at a subsequent time point; and determining a change in ratio between the initial ratio and the subsequent ratio; and
vi) correlating said change in ratio to the parameter of interest,
and/or
B. if the parameter of interest is a spatial parameter, determining the effect of the mutation of interest on the parameter of interest comprises the steps of:
v) defining and/or spatially separating subpopulations of cells on the basis of said spatial parameter of interest in each subpopulation, preferably wherein the spatial parameter of interest is different in each subpopulation;
vi) determining, for each subpopulation, a ratio of cells in which the mutation of interest has been introduced in the target nucleic acid sequence to cells in which the synonymous mutation has been introduced in the target nucleic acid sequence; and
vii) correlating said ratio to the measured spatial parameter of interest for each subpopulation;
thereby assessing the effect of the mutation on the parameter of interest.

2. The method according to claim 1, wherein step A.v) further comprises a step of determining an initial frequency of cells with an indel in the target nucleic acid sequence which is different from the mutation of the first and the second oligonucleotides, wherein the initial frequency of cells is determined at said initial time point, and determining a subsequent frequency of cells with an indel in the target nucleic acid sequence which is different from the mutation of the first and the second oligonucleotides, wherein the subsequent frequency of cells is determined at said subsequent time point.

3. The method according to any one of the preceding claims, wherein step B.vi) further comprises a step of, for each subpopulation, determining a frequency of cells with an indel in the target nucleic acid sequence which is different from the mutation of the first and the second oligonucleotides.

4. The method according to any one of the preceding claims, wherein the mixed population also comprises cells in which the mutation of the first or the second oligonucleotides has been introduced, and/or wherein one or more indel mutations have been introduced in the target nucleic acid sequence.

5. The method according to claim 4, wherein the initial and the subsequent frequency of cells with an indel is further subdivided into, respectively, an initial and a subsequent frequency of cells with an indel resulting in a frameshift mutation and an initial and a subsequent frequency of cells with an indel not resulting in a frameshift mutation, wherein a subsequent frequency of cells with an indel resulting in frameshift mutation lower or higher than the initial frequency of cells with an indel resulting in frameshift mutation indicates that the frameshift indels are affecting the parameter of interest.

6. The method according to claim 5, wherein the frequency of cells with an indel is further subdivided into a frequency of cells with an indel resulting in frameshift mutations and a frequency of cells with an indel not resulting in a frameshift mutation, wherein a frequency of cells with an indel resulting in frameshift mutation in one subpopulation is substantially different from the frequency of cells with an indel resulting in frameshift mutations in a second subpopulation indicates that the frameshift indels are affecting the cells.

7. The method according to any one of the preceding claims, wherein said first oligonucleotide consists of or comprises a stretch of nucleotides identical to said second oligonucleotide except for said mutation of interest or wherein said second oligonucleotide consists of or comprises a stretch of nucleotides identical to said first oligonucleotide except for said synonymous mutation.

8. The method according to any one of the preceding claims, wherein the synonymous mutation of said second oligonucleotide is located in the same genomic position as the mutation of interest in said first oligonucleotide.

9. The method according to any one of claims 1 to 6, wherein the synonymous mutation of said second oligonucleotide is located in a different genomic position from the mutation of interest in said first oligonucleotide.

10. The method according to any one of claims 1 to 6, wherein the synonymous mutation of said second oligonucleotide is located within 20 nucleotides, such as within 19 nucleotides, such as within 18 nucleotides, such as within 17 nucleotides, such as within 16 nucleotides, such as within 15 nucleotides, such as within 14 nucleotides, such as within 13 nucleotides, such as within 12 nucleotides, such as within 11 nucleotides, such as within 10 nucleotides, such as within 9 nucleotides, such as within 8 nucleotides, such as within 7 nucleotides, such as within 6 nucleotides, such as within 5 nucleotides, such as within 4 nucleotides, such as within 3 nucleotides, such as within 2 nucleotides, such as within 1 nucleotide from the position of the mutation of interest in said first oligonucleotide.

11. The method according to any one of the preceding claims, wherein the first and the second oligonucleotides are of different lengths.

12. The method according to any one of the preceding claims, wherein the first and the second oligonucleotides are of the same length.

13. The method according to any one of the preceding claims, wherein the parameter of interest is a temporal parameter of interest, and wherein:
A. If the initial ratio of cells is lower than the subsequent ratio of cells, the mutation is characterised as a mutation having a positive effect on the temporal parameter;
B. If the initial ratio of cells is greater than the subsequent ratio of cells, the mutation is characterised as a mutation having a negative effect on the temporal parameter;
C. If the initial and the subsequent ratio of the cells are substantially the same, the mutation is characterised as a mutation having no effect on the temporal parameter.

14. The method according to any one of the preceding claims, wherein the temporal parameter is selected from the group consisting of cell proliferation, cell growth, anchorage-independent cell growth, contribution to tumor growth, fitness, cell motility, cell invasiveness, cellular metabolism, DNA damage, expression levels of pre-defined genes and/or proteins, resistance to a compound, sensitivity to a compound, production of a compound, anoikis, senescence, contact inhibition and apoptosis.

15. The method according to any one of the preceding claims, wherein step B.v) comprises defining at least one subpopulation of cells and a reference subpopulation of cells from the mixed population on the basis of the spatial parameter of interest and wherein:
A. If the ratio of cells in said subpopulation is greater than in said reference subpopulation, the mutation is characterised as a mutation having a positive effect on the spatial parameter;
B. If the ratio of cells in said subpopulation is lower than the ratio of said reference subpopulation, the mutation is characterised as a mutation having a negative effect on the spatial parameter;
C. If the ratio of cells in said subpopulation is substantially the same as the ratio of cells in said reference subpopulation, the mutation is characterised as a mutation having no effect on the spatial parameter.

16. The method according to claim 15, wherein step B.v) further comprises spatially separating said at least one subpopulation from the reference subpopulation.

17. The method according to any one of the preceding claims, wherein the spatial parameter is selected from the group consisting of: cell proliferation, cell growth, fitness, cell motility, cell invasiveness, cellular metabolism, cell differentiation, DNA damage, expression levels of pre-defined genes and/or proteins, resistance to a compound, sensitivity to a compound, production of a compound, anoikis, senescence, apoptosis, DNA methylation, and protein post-translational modification.

18. The method according to any one of claims 15 to 17, wherein the spatial parameter of interest has a value of interest defined by comparison with a reference value of the same spatial parameter in a reference population.

19. The method according to any one of the preceding claims, wherein steps iii) and iv), and optionally steps A.v) to A.vi) and/or steps B.v) to B.vii), are performed more than once for further predetermined duration(s).

20. The method according to any one of the preceding claims, wherein step ii) further comprises selecting the cells in which one of the first oligonucleotide or the second oligonucleotide has been introduced, thereby obtaining a subpopulation of cells comprising cells in which the mutation of interest has been introduced in the target nucleic acid sequence, and cells in which the synonymous mutation has been introduced in the target nucleic acid sequence.

21. The method according to any one of the preceding claims, wherein step ii) further comprises selecting the cells in which the nuclease or the polynucleotide encoding said nuclease has been introduced, thereby obtaining a subpopulation of cells enriched in cells in which the mutation of interest and/or the synonymous mutation has been introduced in the target nucleic acid sequence.

22. The method according to any one of the preceding claims, wherein the first oligonucleotide comprises at least one mutation of interest within the binding region of the nuclease, and wherein the second oligonucleotide comprises at least one synonymous mutation within the same binding region of the nuclease.

23. The method according to any one of the preceding claims, wherein the first oligonucleotide comprises at least one first mutation, which is a synonymous mutation lying inside the binding region of the nuclease, and further comprises at least one second mutation, which is a non-synonymous mutation of interest lying outside the binding region of the nuclease, and wherein the second oligonucleotide comprises at least one synonymous mutation lying within the same region as the first mutation and further comprises at least one further synonymous mutation lying inside the same region as the second mutation, wherein the first mutation and the synonymous mutation when introduced in the target nucleic acid sequence prevent the nuclease and the targeting means from binding to and/or generating a further SSB or a further DSB in the resulting nucleic acid sequence, preferably wherein the first mutation and the synonymous mutation are identical.

24. The method according to any one of the preceding claims, wherein the first oligonucleotide and the second oligonucleotide differ only in the location of the mutation of interest.

25. The method according to any one of the preceding claims, wherein the method is performed in vivo or in vitro.

26. The method according to any one of the preceding claims, wherein the first oligonucleotide is a plurality of first oligonucleotides each comprising a pre-determined mutation of interest and wherein the second oligonucleotide is a plurality of second oligonucleotides each comprising a pre-determined synonymous mutation.

27. The method according to claim 26, wherein the first oligonucleotide comprises a plurality of pre-determined mutations of interest and wherein the second oligonucleotide comprises a plurality of pre-determined synonymous mutations.

28. The method according to claim 27, wherein the plurality of mutations is at least 2 different mutations, such as at least 3 different mutations, such as at least 4 different mutations, such as at least 5 different mutations, such as at least 10 different mutations, such as at least 20 different mutations, such as at least 50 different mutations.

29. The method according to any one of claims 26 to 28, wherein said second oligonucleotides are otherwise identical to said first oligonucleotides.

30. The method according to any one of the preceding claims, wherein the cell is a vertebrate cell, an invertebrate cell, a plant cell, a yeast cell, a fungal cell or a bacterial cell.

31. The method according to any one of the preceding claims, wherein the cell is a human cell.

32. The method according to any one of the preceding claims, wherein the target nucleic acid sequence is within a gene, a promoter or an enhancer of a gene, wherein the gene is or is suspected to be an oncogene, a proto-oncogene, a tumour suppressor gene, a gene encoding an enzyme such as an enzyme involved in the production of a compound such as a metabolite, a resistance gene, such as a gene involved in resistance to a compound, a pharmaceutical compound or a pathogen such as a virus, a gene encoding a protein involved in cellular fitness and/or growth, a gene encoding a protein for any cell function, a microRNA or a long non-coding RNA.

33. The method according to any one of the preceding claims, wherein the nuclease comprises or consists of a CRISPR/Cas nuclease and the targeting means comprise or consist of a guide RNA capable of hybridizing to the genomic target region.

34. The method according to claim 33, wherein the CRISPR/Cas nuclease is Streptococcus pyogenes Cas9, such as the Streptococcus pyogenes Cas9 nuclease of SEQ ID NO: 1, the human, codon-optimized Streptococcus pyogenes Cas9 (D10A) nickase, such as the human, codon-optimized 10 Streptococcus pyogenes Cas9 (D10A) nickase of SEQ ID NO: 2, Francisella novicida Cas12a, such as the Francisella novicida Cas12a nuclease of SEQ ID NO: 3 or MAD7, or functional variants thereof which retain nuclease and/or nickase activity.

35. The method according to claim 33, wherein the CRISPR/Cas nuclease is Cas9-NG, such as the Cas9-NG nuclease of SEQ ID NO: 5.

36. The method according to claim 33, wherein the CRISPR/Cas nuclease is a prime editor nuclease, such as the prime editor PE2 of SEQ ID NO: 9.

37. The method according to claim 36, wherein the guide RNA capable of hybridizing to the genomic target region comprises the first and/or the second oligonucleotide.

38. The method according to any one of the preceding claims, wherein the synonymous mutation is a single base change compared to the genomic target region.

39. The method according to any one of the preceding claims, wherein the mutation of interest is a single base change compared to the genomic target region.

40. The method according to any one of the preceding claims wherein the step of determining the ratio of cells of step A.v) and/or step B.vi) is performed for only a fraction of the cell population and/or only a fraction of each subpopulation.

41. The method according to any one of the preceding claims, wherein step A.v) and/or step B.vi) comprises amplifying a region comprising the target nucleic acid sequence, such as by PCR, to produce an amplicon comprising the target nucleic acid sequence, optionally followed by sequencing, such as next-generation sequencing, of said amplicon.

42. The method according to claim 41, wherein the amplification is performed using primers that anneal outside the region substantially identical or complementary to the first or second oligonucleotides.

43. The method according any one of the preceding claims, wherein the determined duration is at least 2 hours, such as at least 4 hours, such as at least 8 hours, such as at least 12 hours, such as at least 18 hours, such as at least 24 hours, such as at least 48 hours, such as at least 72 hours.

44. The method according any one of the preceding claims, wherein the determined duration is at least such as at least 1 week, such as at least 2 weeks, such as at least 3 weeks, such as at least 4 weeks, such as at least 2 months, such as at least 4 months, such as at least 6 months, such as at least 8 months, such as at least 10 months, such as at least 12 months, such as at least 1½ year, such as at least 2 years.

45. The method according any one of the preceding claims, wherein the time between the initial time point and the subsequent time point is at least 4 hours, such as at least 8 hours, such as at least 12 hours, such as at least 18 hours, such as at least 24 hours, such as at least 48 hours, such as at least 72 hours.

46. The method according any one of the preceding claims, wherein the time between the initial time point and the subsequent time point is at least 1 week, such as at least 2 weeks, such as at least 3 weeks, such as at least 4 weeks, such as at least 2 months, such as at least 4 months, such as at least 6 months, such as at least 8 months, such as at least 10 months, such as at least 12 months, such as at least 1½ years, such as at least 2 years.

47. The method according to any one of the preceding claims, wherein step B.v) comprises a step of spatially separating the cells to separate the mixed cell population into subpopulations on the basis of a cell marker, such as the presence or absence or a graded level of a cell marker, for example a cell marker for cell proliferation, cell growth, fitness, cell motility, cell invasiveness, cellular metabolism, cell differentiation, DNA damage, expression levels of pre-defined genes or proteins, resistance to a compound, sensitivity to a compound, production of a compound, anoikis, senescence, apoptosis, DNA methylation or protein post-translational modification, optionally wherein the cell marker is the spatial parameter of interest.

48. The method according to claim 47, wherein said step of spatially separating the cells is performed by FACS.

49. The method according to claim 48, wherein step B.v) further comprises a step of staining the cells with an antibody marker, such as a fluorescently labelled antibody, prior to FACS-sorting the cells, whereby cells may be spatially separated based on the signal intensity of the antibody marker.

50. The method according to any one of the preceding claims, wherein step B.v) comprises defining the subpopulations on the basis of a cell property, such as the ability of the cells to migrate or invade, optionally wherein the cell property is the spatial parameter of interest.

51. The method according to any one of the preceding claims, wherein each subpopulation of step B.v) comprises one or more cells, such as a single cell or a plurality of cells.

52. The method according to any one of the preceding claims, wherein the parameter of interest is a cellular response to a compound or an external stimulus, such as temperature, drought or pressure, and wherein the medium of step iii) comprises said compound or step iii) additionally comprises subjecting the cell population to the external stimulus, wherein the cellular response is a temporal parameter or a spatial parameter.

53. The method according to claim 52, wherein the compound is a therapeutic agent or a candidate therapeutic agent, a virus or a viral agent, a pathogen, an active agent, a metabolite or a cell signaling molecule.

54. The method according to claim 52, wherein the external stimulus is a physical stimulus.

55. The method according to any one of claims 52 to 54, wherein the cellular response is monitored as a temporal parameter, and wherein the initial ratio of cells being lower than the subsequent ratio of cells indicates a positive effect of the mutation of interest on the cellular response to the compound; the initial ratio of cells being greater than the subsequent ratio of cells indicates a negative effect of the mutation of interest on the cellular response to the compound; and substantially no difference between the initial ratio of cells and the subsequent ratio of cells indicates no effect of the mutation of interest on the cellular response to the compound.

56. The method according to any one of claims 52 to 55, wherein the cellular response is monitored as a spatial parameter, wherein for each subpopulation, a ratio of said subpopulation lower than the ratio of the reference subpopulation indicates that the mutation has a negative effect on the cellular response to the compound; a ratio of said subpopulation greater than the ratio of the reference subpopulation indicates that the mutation has a positive effect on the cellular response to the compound; and a ratio of said subpopulation around the ratio of the reference subpopulation indicates that the mutation has no effect on the cellular response to the compound.

57. The method according to any one of the preceding claims, wherein one or more of the first oligonucleotide, the second oligonucleotide, the targeting means and the polynucleotide encoding the nuclease are comprised within one or more vectors or within a virus.

58. The method according to any one of the preceding claims wherein the first oligonucleotide and/or the second oligonucleotide is single-stranded.

59. The method according to any one of the preceding claims wherein the first oligonucleotide and/or the second oligonucleotide is double-stranded.

60. The method according to any one of the preceding claims wherein the first oligonucleotide and/or the second oligonucleotide is modified, such as by introduction of one or more phosphorothioate bonds to inhibit oligonucleotide degradation by nucleases.

61. A system comprising:
i. a nuclease or a polynucleotide encoding said nuclease, wherein the nuclease is capable of generating one or more single-strand breaks (SSBs) or double-strand breaks (DSBs) in a target nucleic acid sequence, and targeting means directing the nuclease to the target nucleic acid sequence, whereby the nuclease is capable of binding to a binding region of the target nucleic acid sequence, and whereby the nuclease is capable of generating one or more single-stranded breaks (SSBs) or double-strand breaks (DSBs) in the target nucleic acid sequence;
ii. a first oligonucleotide comprising a mutation of interest, preferably a non-silent mutation, preferably a non-synonymous mutation, wherein said non-synonymous mutation introduces a change in the encoded amino acid sequence compared to the amino acid sequence encoded by the target nucleic acid sequence, and otherwise identical to or complementary to said target nucleic acid sequence, wherein said mutation of interest preferably lies within the binding region of said nuclease; and
iii. a second oligonucleotide comprising a synonymous mutation, wherein said synonymous mutation preferably lies within the binding region of said nuclease, and wherein said synonymous mutation introduces no change in the encoded amino acid sequence compared to the amino acid sequence encoded by the target nucleic acid sequence,
wherein i., ii., and iii. are comprised within the same cell population.

62. The system according to claim 61, wherein the first oligonucleotide is a plurality of first oligonucleotides each comprising a mutation of interest and wherein the second oligonucleotide is a plurality of second oligonucleotides each comprising a synonymous mutation.

63. The system according to any one of claims 61 to 62, wherein the first oligonucleotide comprises a plurality of pre-determined mutations of interest and wherein the second oligonucleotide comprises a plurality of pre-determined synonymous mutations.

64. The system according to any one of claims 62 to 63, wherein said second oligonucleotides are otherwise identical to said first oligonucleotides.

65. The system according to any one of claims 61 to 64, wherein the plurality of mutations is at least 2 different mutations, such as at least 3 different mutations, such as at least 4 different mutations, such as at least 5 different mutations, such as at least 10 different mutations, such as at least 20 different mutations, such as at least 50 different mutations.

66. The system according to any one of claims 61 to 65, wherein the target nucleic acid sequence is within a gene, a promoter or an enhancer of a gene, wherein the gene is or is suspected to be an oncogene, a proto-oncogene, a tumor suppressor gene, a gene encoding an enzyme such as an enzyme involved in the production of a compound such as a metabolite, a resistance gene, such as a gene involved in resistance to a compound, a pharmaceutical compound or a pathogen such as a virus, a gene encoding a protein involved in cellular fitness and/or growth, a gene encoding a protein for any cell function, a microRNA or a long non-coding RNA.

67. The system according to any one of claims 61 to 66, wherein the nuclease comprises or consist of a CRISPR/Cas nuclease and the targeting means comprise or consist of a guide RNA capable of hybridizing to the target nucleic acid sequence.

68. The system according to claim 67, wherein the CRISPR/Cas nuclease is Streptococcus pyogenes Cas9, such as the Streptococcus pyogenes Cas9 nuclease of SEQ ID NO: 1, the human, codon-optimized Streptococcus pyogenes Cas9 (D10A) nickase, such as the human, codon-optimized Streptococcus pyogenes Cas9 (D10A) nickase of SEQ ID NO: 2, Francisella novicida Cas12a, such as the Francisella novicida Cas12a nuclease of SEQ ID NO: 3 or MAD7, or functional variants thereof which retain nuclease and/or nickase activity.

69. The system according to claim 68, wherein the CRISPR/Cas nuclease is Cas9-NG, such as the Cas9-NG nuclease of SEQ ID NO: 5.

70. The system according to claim 69, wherein the CRISPR/Cas nuclease is a prime editor, such as the prime editor PE2 of SEQ ID NO: 9.

71. The system according to claim 70, wherein the guide RNA capable of hybridizing to the genomic target region comprises the first and/or the second oligonucleotide.

72. The system according to any one of claims 61 to 71, wherein the synonymous mutation is a single base change compared to the target nucleic acid sequence.

73. The system according to any one of claims 61 to 72, wherein the mutation of interest is a single base change compared to the target nucleic acid sequence.

74. The system according to any one of claims 61 to 73, further comprising primers for amplifying a region comprising the target nucleic acid sequence, such as a forward and a reverse primer allowing amplification by PCR.

75. The system according to claim 74, wherein said primers anneal outside the region substantially identical or complementary to the first or second oligonucleotides.

76. The system according to any one of claims 61 to 75, wherein one or more of the first oligonucleotide, the second oligonucleotide, the targeting means and the polynucleotide encoding the nuclease are comprised within one or more vectors, such as one or more plasmids.

77. A population of host cells comprising the system according to any one of claims 61 to 76.

78. The population of host cells according to claim 77, wherein the host cell is a bacterial cell or a eukaryotic cell such as a vertebrate cell, an invertebrate cell, a plant cell, a yeast cell or a fungal cell.

79. Use of the system according to any one of claims 61 to 76 in a method for assessing the effects of a mutation of interest in a cell, preferably wherein the method is according to any one of claims 1 to 60.

80. The use according to claim 79, wherein the cell is a mammalian cell such as a human cell, a vertebrate cell, an invertebrate cell, a plant cell, a yeast cell or a fungal cell.

81. The use according to any one of claims 79 to 80, wherein the method is performed in vitro or in vivo.


SEQUENCE LISTING (10 sequences)

SEQ ID NO: 1 - protein, 1368 aa, Streptococcus pyogenes [sequence omitted]

SEQ ID NO: 2 - protein, 1399 aa, Artificial
  Human, codon-optimized (D10A) nickase
  MISC_FEATURE (1)..(1399): Sequence is in silico translation of Cas9-D10A in the plasmid pCas9n-sgPdx1
MYPYDVPDYASPKKKRKVEASDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVEAS

SEQ ID NO: 3 - protein, 1300 aa, Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 4 - DNA, 4104 bp, Artificial
  Cas9-NG
  misc_feature (1)..(4104): Sequence derived from Cas9-NG in the plasmid pX330-SpCas9-NG
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatccggcccaagaggaacagcgataagctgatcgccagaaagaaggactgggaccctaagaagtacggcggcttcgttagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgcccggttcctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctcgggccttcaagtactttgacaccaccatcgaccggaaggtgtaccggagcaccaaagaggtgctggacgccaccctgatccaccagagcatcaccggcctgtacgagacacggatcgacctgtctcagctgggaggcgac

SEQ ID NO: 5 - protein, 1368 aa, Artificial
  Cas9-NG
  MISC_FEATURE (1)..(1368): In silico translation based on DNA sequence of Cas9-NG in the plasmid pX330-SpCas9-NG
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 6 - DNA, 4104 bp, Streptococcus pyogenes
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 7 - DNA, 4104 bp, Artificial
  Human, codon-optimized (D10A) nickase
  misc_feature (1)..(4104): Sequence is Cas9-D10A in the plasmid pCas9n- sgPdx1
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 8 - DNA, 3960 bp, Francisella novicida
atgtcaatttatcaagaatttgttaataaatatagtttaagtaaaactctaagatttgagttaatcccacagggtaaaacacttgaaaacataaaagcaagaggtttgattttagatgatgagaaaagagctaaagactacaaaaaggctaaacaaataattgataaatatcatcagttttttatagaggagatattaagttcggtttgtattagcgaagatttattacaaaactattctgatgtttattttaaacttaaaaagagtgatgatgataatctacaaaaagattttaaaagtgcaaaagatacgataaagaaacaaatatctgaatatataaaggactcagagaaatttaagaatttgtttaatcaaaaccttatcgatgctaaaaaagggcaagagtcagatttaattctatggctaaagcaatctaaggataatggtatagaactatttaaagccaatagtgatatcacagatatagatgaggcgttagaaataatcaaatcttttaaaggttggacaacttattttaagggttttcatgaaaatagaaaaaatgtttatagtagcaatgatattcctacatctattatttataggatagtagatgataatttgcctaaatttctagaaaataaagctaagtatgagagtttaaaagacaaagctccagaagctataaactatgaacaaattaaaaaagatttggcagaagagctaacctttgatattgactacaaaacatctgaagttaatcaaagagttttttcacttgatgaagtttttgagatagcaaactttaataattatctaaatcaaagtggtattactaaatttaatactattattggtggtaaatttgtaaatggtgaaaatacaaagagaaaaggtataaatgaatatataaatctatactcacagcaaataaatgataaaacactcaaaaaatataaaatgagtgttttatttaagcaaattttaagtgatacagaatctaaatcttttgtaattgataagttagaagatgatagtgatgtagttacaacgatgcaaagtttttatgagcaaatagcagcttttaaaacagtagaagaaaaatctattaaagaaacactatctttattatttgatgatttaaaagctcaaaaacttgatttgagtaaaatttattttaaaaatgataaatctcttactgatctatcacaacaagtttttgatgattatagtgttattggtacagcggtactagaatatataactcaacaaatagcacctaaaaatcttgataaccctagtaagaaagagcaagaattaatagccaaaaaaactgaaaaagcaaaatacttatctctagaaactataaagcttgccttagaagaatttaataagcatagagatatagataaacagtgtaggtttgaagaaatacttgcaaactttgcggctattccgatgatatttgatgaaatagctcaaaacaaagacaatttggcacagatatctatcaaatatcaaaatcaaggtaaaaaagacctacttcaagctagtgcggaagatgatgttaaagctatcaaggatcttttagatcaaactaataatctcttacataaactaaaaatatttcatattagtcagtcagaagataaggcaaatattttagacaaggatgagcatttttatctagtatttgaggagtgctactttgagctagcgaatatagtgcctctttataacaaaattagaaactatataactcaaaagccatatagtgatgagaaatttaagctcaattttgagaactcgactttggctaatggttgggataaaaataaagagcctgacaatacggcaattttatttatcaaagatgataaatattatctgggtgtgatgaataagaaaaataacaaaatatttgatgataaagctatcaaagaaaataaaggcgagggttataaaaaaattgtttataaacttttacctggcgcaaataaaatgttacctaaggttttcttttctgctaaatctataaaattttataatcctagtgaagatatacttagaataagaaatcattccacacatacaaaaaatggtagtcctcaaaaaggatatgaaaaatttgagtttaatattgaagattgccgaaaatttatagatttttataaacagtctataagtaagcatccggagtggaaagattttggatttagattttctgatactcaaagatataattctatagatgaattttatagagaagttgaaaatcaaggctacaaactaacttttgaaaatatatcagagagctatattgatagcgtagttaatcagggtaaattgtacctattccaaatctataataaagatttttcagcttatagcaaagggcgaccaaatctacatactttatattggaaagcgctgtttgatgagagaaatcttcaagatgtggtttataagctaaatggtgaggcagagcttttttatcgtaaacaatcaatacctaaaaaaatcactcacccagctaaagaggcaatagctaataaaaacaaagataatcctaaaaaagagagtgtttttgaatatgatttaatcaaagataaacgctttactgaagataagtttttctttcactgtcctattacaatcaattttaaatctagtggagctaataagtttaatgatgaaatcaatttattgctaaaagaaaaagcaaatgatgttcatatattaagtatagatagaggtgaaagacatttagcttactatactttggtagatggtaaaggcaatatcatcaaacaagatactttcaacatcattggtaatgatagaatgaaaacaaactaccatgataagcttgctgcaatagagaaagatagggattcagctaggaaagactggaaaaagataaataacatcaaagagatgaaagagggctatctatctcaggtagttcatgaaatagctaagctagttatagagtataatgctattgtggtttttgaggatttaaattttggatttaaaagagggcgtttcaaggtagagaagcaggtctatcaaaagttagaaaaaatgctaattgagaaactaaactatctagttttcaaagataatgagtttgataaaactgggggagtgcttagagcttatcagctaacagcaccttttgagacttttaaaaagatgggtaaacaaacaggtattatctactatgtaccagctggttttacttcaaaaatttgtcctgtaactggttttgtaaatcagttatatcctaagtatgaaagtgtcagcaaatctcaagagttctttagtaagtttgacaagatttgttataaccttgataagggctattttgagtttagttttgattataaaaactttggtgacaaggctgccaaaggcaagtggactatagctagctttgggagtagattgattaactttagaaattcagataaaaatcataattgggatactcgagaagtttatccaactaaagagttggagaaattgctaaaagattattctatcgaatatgggcatggcgaatgtatcaaagcagctatttgcggtgagagcgacaaaaagttttttgctaagctaactagtgtcctaaatactatcttacaaatgcgtaactcaaaaacaggtactgagttagattatctaatttcaccagtagcagatgtaaatggcaatttctttgattcgcgacaggcgccaaaaaatatgcctcaagatgctgatgccaatggtgcttatcatattgggctaaaaggtctgatgctactaggtaggatcaaaaataatcaagagggcaaaaaactcaatttggttatcaaaaatgaagagtattttgagttcgtgcagaataggaataactaattcattcaagaatatattaccctgtcagtttagcgactattacctctttaataattt

SEQ ID NO: 9 - protein, 2092 aa, Artificial
  PE2 (prime editor)
  misc_feature (1)..(2092): PE2
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFE

SEQ ID NO: 10 - DNA, 6279 bp, Artificial
  PE2 (primer editor) DNA
  misc_feature (1)..(6279): PE2 (DNA)
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagtaa